(19) United States
(12) Patent Application Publication
(10) Pub. No.: US 2008/0085281 A1
(43) Pub. Date: Apr. 10, 2008

(54) Inhibitors of Dkk-1

(75) Inventors: Darwin Prockop, New Orleans, LA (US); Carl Gregory, New Orleans, LA (US); William Gunn, Hattiesburg, MS (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: The Administrators of the Tulane Educational Fund, New Orleans, LA (US)

(21) Appl. No.: 11/828,145

(22) Filed: Jul. 25, 2007

Related U.S. Application Data
(62) Division of application No. 10/839,515, filed on May 5, 2004, now abandoned.
Application No. 10/839,515 is a continuation-in-part of application No. 10/830,352, now abandoned.
Application No. 10/830,352 is a continuation-in-part of application No. 10/442,506, filed on May 21, 2003.

(51) Int. Cl. A61K 39/395; A61K 38/00; A61P 17/02

(52) U.S. Cl. 424/146.1; 514/12

(57) ABSTRACT

The present invention encompasses methods and compositions for enhancing the growth of adult marrow stromal cells. The present invention also encompasses methods and compositions for regulating the effects of Dkk-1. Methods and compositions for treatment of osteolytic lesions in multiple myeloma and enhancing osteogenesis are also included.

### CROSS REFERENCE TO RELATED APPLICATIONS

This is a division of application Ser. No. 10/839,515, filed May 5, 2004, which is a continuation-in-part of application Ser. No. 10/830,352, filed on Apr. 22, 2004, which is a continuation-in-part of application Ser. No. 10/442,506, filed on May 21, 2003, all of which are incorporated herein by reference.

### STATEMENT REGARDING FEDERAL SUPPORT FOR RESEARCH AND DEVELOPMENT

The present invention was made in part with support from grants obtained from the National Institutes of Health (Nos. AR48323, AR47796, and AR47161). The federal government may have rights in the present invention.

### BACKGROUND OF THE INVENTION

Bone marrow contains at least two types of stem cells, hematopoietic stem cells and stem cells for non-hematopoietic tissues variously referred to as mesenchymal stem cells or marrow stromal cells (MSCs). MSCs are of interest because they are easily isolated from a small aspirate of bone marrow, they readily generate single-cell derived colonies. The single-cell derived colonies can be expanded through as many as 50 population doublings in about 10 weeks, and they can differentiate into osteoblasts, adipocytes, chondrocytes (A. J. Friedenstein, et al. Cell Tissue Kinet. 3:393-403 (1970); H. Castro-Malaspina et al., Blood 56:289-301 (1980); N. N. Beresford, et al. J. Cell Sci. 102:341-351 (1992); D. J. Prockop, Science 276:71-74 (1997)), myocytes (S. Wakitani, et al. Muscle Nerve 18:1417-1426 (1995)), astrocytes, oligodendrocytes, and neurons (S. A. Azizi, et al. Proc. Natl. Acad. Sci. USA 95:3908-3913 (1998); G. C. Kopen, et al. Proc. Natl. Acad. Sci. USA 96:10711-10716 (1999); M. Chopp et al., Neuroreport II, 3001-3005 (2000); D. Woodbury, et al. Neuroscience Res. 61:364-370 (2000)).

Furthermore, MSCs can give rise to cells of all three germ layers (Kopen, G. C. et al., Proc. Natl. Acad. Sci. 96:10711-10716 (1999); Liechty, K. W. et al. Nature Med. 6:1282-1286 (2000); Kotton, D. N. et al. Development 128:5181-5188 (2001); Toma, C. et al. Circulation 105:93-98 (2002); Jiang, Y. et al. Nature 418:41-49 (2002). In vivo evidence indicates that unfractionated bone marrow-derived cells as well as pure populations of MSCs can give rise to epithelial cell-types including those of the lung (Krause, et at. Cell 105:369-377 (2001); Petersen, et al. Science 284:1168-1170 (1999)) and several recent studies have shown that engraftment of MSCs is enhanced by tissue injury (Ferrari, G. et al. Science 279:1528-1530 (1998); Okamoto, R. et al. Nature Med. 8:1101-1017 (2002)). For these reasons, MSCs are currently being tested for their potential use in cell and gene therapy of a number of human diseases (Horwitz et al., Nat. Med. 5:309-313 (1999); Caplan, et al. Clin. Orthoped. 379:567-570 (2000)).

Marrow stromal cells constitute an alternative source of pluripotent stem cells. Under physiological conditions they are believed to maintain the architecture of bone marrow and regulate hematopoiesis with the help of different cell adhesion molecules and the secretion of cytokines, respectively (Clark, B. R. & Keating, A. (1995) Ann NY Acad Sci 770:70-78). MSCs grown out of bone marrow cell suspensions by their selective attachment to tissue culture plastic can be efficiently expanded (Azizi, S. A., et al. (1998) Proc Natl Acad Sci USA 95:3908-3913; Colter, D. C., et al. (2000) Proc Natl Acad Sci USA 97:3213-218) and genetically manipulated (Schwarz, E. J., et al. (1999) Hum Gene Ther 10:2539-2549).

MSC are referred to as mesenchymal stem cells because they are capable of differentiating into multiple mesodermal tissues, including bone (Beresford, J. N., et al. (1992) J Cell Sci 102:341-351), cartilage (Lennon, D. P., et al. (1995) Exp Cell Res 219:211-222), fat (Beresford, J. N., et al. (1992) J Cell Sci 102, 341-351) and muscle (Wakitani, et al. (1995) Muscle Nerve 18:1417-1426). In addition, differentiation into neuron-like cells expressing neuronal markers has been reported (Woodbury, D., et al. (2000) J Neurosci Res 61:364-370; Sanchez-Ramos, J., et al. (2000) Exp Neurol 164:247-256; Deng, W., et al. (2001) Biochem Biophys Res Commun 282:148-152), suggesting that MSC may be capable of overcoming germ layer commitment.

In order to use MSCs for cell and gene therapy applications, large numbers of the cells are produced in vitro for transfection. One problem with repeated culture of MSCs is that the MSCs may lose their proliferative capacity, and their potential to differentiate into various lineages.

The replication rate of the MSCs is sensitive to initial plating density. Previously, it has been observed that human MSCs proliferate most rapidly and retain their multipotentiality if the MSCs are plated at very low densities of about 3 cells per square centimeter (Colter, et al., PNAS 97:3213-3218 (2000)). However, many other variables must be considered when selecting culture conditions. In particular, yield and quality of MSCs obtained from bone marrow aspirates varies widely because MSCs populations are generally heterogeneous, even when they are cultured as single-cell derived colonies. Small, rapidly self-renewing cells (RS cells), which are a subpopulation of MSCs having the highest multipotentiality, are gradually replaced by flat MSCs (called mMSCs), which have low multipotentiality, as the MSCs population expands, leading to heterogeneity.

The Wnt signaling pathway controls patterning and cell fate determination in the development of a wide range of organisms (Cadigan et al., 1997, Genes Dev. 11:3286-3305). The signaling can occur by different pathways (Huelsken et al., 2001, Curr. Opin. Genet. Dev. 11:547-553). The Wnt signaling pathway is activated by the interaction between secreted Wnts and their receptors, the frizzled proteins (Hisken et al. 2000, J. Cell. Sci. 113:3545-3546), with the LDL receptor-related proteins LRP5 and LRP6 acting as co-receptors. The downstream effects of Wnt signaling include activation of Disheveled (Dvl1) protein, resulting in the activation and subsequent recruitment of Akt to the Axin-β-catenin-G5K₃β-APC complex (Fukumoto et al., 2001 J. Biol. Chem. 276:17479-17483). This is followed by the phosphorylation and inactivation of GSK3β, resulting in inhibition of phosphorylation and degradation of β-catenin. The accumulated β-catenin is translocated to the nucleus where it interacts with transcription factors of the lymphoid enhancer factor-T cell factor (LEF/TCF) family and induces the transcription of target genes.

Lung, breast, prostate cancer and multiple myeloma have an affinity for bone, where they cause osteoblastic lesions or osteolytic lesions (Mundy. 2002 Nat Rev Cancer 2:584-593). Research on the mechanisms by which multiple myeloma cells induce osteolysis has focused on the osteoclast's role in shifting the normal balance between bone formation and bone resorption in favor of resorption (Roodman 2001 J Clin Oncol 19:3562-3571). Indeed, the number and function of osteoblasts are decreased in myeloma with osteolytic lesions (Bataille et al. 1986 Br J Cancer 53:805-810; Bataille et al. 1991 J Clin Invest 88:62-66; Bataille et al. 1990 Br J Haematol 76:484-487; Taube et al. 1992 Eur J Haematol 49:192-198.

Osteolytic bone lesions are by far the most common skeletal manifestations in patients with myeloma. Although the precise molecular mechanisms remain unclear, it is observed that 1) The mechanism by which bone is destroyed in myeloma is via the osteociast, the normal bone-resorbing cell; 2) Osteoclasts accumulate on bone-resorbing surfaces in myeloma adjacent to collections of myeloma cells and it appears that the mechanism by which osteoclasts are stimulated in myeloma is a local one; 3) Cultures of human myeloma cells in vitro produce several osteoclast activating factors, including lymphotoxin-alpha (LT-a), interleukin-1 (IL-1), parathyroid-hormone related protein (PTHrP) and interleukin-6 (IL-6); 4) Hypercalcemia occurs in approximately one-third of patients with myeloma some time during the course of the disease. Hypercalcemia is associated with markedly increased bone resorption and frequently with impairment in glomerular filtration; 5) The increase in osteoclastic bone resorption in myeloma is associated with a marked impairment in osteoblast function.

Common causes of localized osteolytic lesions are metastatic bone disease, multiple myeloma and lymphoma. In addition, circumscribed bone defects can be caused by numerous benign bone disorders including, among others, bone cysts, fibrous dyslasia, infections, benign bone tumors and impaired fracture healing. Current treatment of these lesions comprises surgical removal or radiotherapeutic destruction of the pathological tissue, fracture fixation, implant stabilization and the reconstruction of the skeletal defect. However, current surgical methods utilizing autograft or allograft bone to close the skeletal defects have limitations.

Currently, there are no effective means to treat osteolytic lesions in multiple myeloma. The current state of knowledge and practice with respect to the therapy of osteolytic lesions is by no means satisfactory. Thus, it can be appreciated that a superior method for treatment of osteolytic lesions in multiple myeloma would be of great utility. Specifically, there is a need for effective agents that can be used in the diagnosis and therapy of individuals with osteolytic lesions. The present invention satisfies this need.

### BRIEF SUMMARY OF THE INVENTION

The present invention relates to compositions and methods of antagonizing Dkk-1. In one embodiment of the present invention, a Dkk-1 antagonist comprises a peptide corresponding to the LRP-6 binding site within Dkk-1. Preferably, the Dkk-1 antagonist is Peptide A as set forth in SEQ ID NO:11.

In another embodiment of the present invention, a Dkk-1 antagonist can be an antibody that specifically binds to Dkk-1.

The present invention also relates to compositions and methods for treating an osteolytic lesion in a mammal. A further embodiment, relates to compositions and methods for treating an osteolytic lesion in multiple myeloma in a mammal.

The present invention relates to the novel discovery that Dkk-1 antagonist or compositions that inhibit the effects of Dkk-1, for example lithium, can be used to treat an osteolytic lesion. Another aspect of the present invention is the discovery that Dkk-1 antagonists or compositions that inhibit the effects of Dkk-1 can be used to enhance osteogenesis. Preferably, Peptide A is used to administer a mammal in need to treat an osteolytic lesion in multiple myeloma. Also preferably is to use Peptide A as an Dkk-1 antagonist to enhance osteogenesis in a mammal.

In another aspect of the present invention, the compositions of the present invention can be used to inhibit the proliferation of a cell. Preferably, a Dkk-1 antagonist or a composition capable of inhibiting the effects of Dkk-1 can be used to inhibit the proliferation of a cell. More preferably, Peptide A is used as a Dkk-1 antagonist to inhibit the proliferation of a cell.

The present invention relates to various methods for improving culture conditions for bone marrow stromal cells (MSCs) and enhancing growth of MSCs.

In one embodiment, a method for enhancing the multipotentiality of bone marrow stromal cells cultured in vitro is taught. The method includes adding an effective amount of exogenous Dkk-1 to the growth medium in which the MSCs are cultured, thereby enhancing the multipotentiality of said cells.

Preferably, Dkk-1 is added to the growth medium in a range of from about 0.01 microgram per milliliter to about 0.1 microgram per milliliter. In one embodiment present invention, Dkk-1 is added to the growth medium at a concentration of about 0.1 microgram per milliliter.

In another embodiment of the present invention, Dkk-1 is added to the growth medium at a concentration of about 0.01 microgram per milliliter.

A growth medium for culturing bone marrow stromal cells is also an aspect of the present invention. The growth medium includes exogenous Dkk-1. In another embodiment, the growth medium also includes epidermal growth factor, basic fibroblast growth factor, autologous serum, or combinations thereof.

Preferably, Dkk-1 is present in the growth medium in a range of from about 0.01 microgram per milliliter to about 0.1 microgram per milliliter. In one embodiment present invention, Dkk-1 is present in the growth medium at a concentration of about 0.1 microgram per milliliter. In another embodiment of the present invention, Dkk-1 is present in the growth medium at a concentration of about 0.01 microgram per milliliter.

In one embodiment of the present invention, the epidermal growth factor (EGF) and the basic fibroblast growth factor (bFGF) are each present in the growth medium at a range of from about 0.1 nanogram per milliliter to about 100 nanograms per milliliter. In another embodiment of the present invention, the epidermal growth factor (EGF) and the basic fibroblast growth factor (bFGF) are each present in the growth medium at a range of from about 5 nanograms per milliliter to about 20 nanograms per milliliter. In one aspect of the present invention, the EGF and bFGF are present at about 10 nanograms per milliliter.

The present invention also includes compositions and methods of modulating the proliferation of a cell. Preferably, the present invention encompasses methods of enhanced and retarded the proliferation of a cell using the a Dkk-1 agonist to enhance the proliferation of a cell and a Dkk-1 antagonist or a composition capable of inhibiting the effects of Dkk-1 to retard the proliferation of the cell. Another embodiment of the present invention also encompasses the differentiation of a cell using the compositions of the present invention. Preferably, a Dkk-1 agonist can be used to

The present invention also includes a method of enhancing the growth rate of bone marrow stromal cells in vitro. The method includes plating the bone marrow stromal cells at an initial density of at least about 50 cells per square centimeter, but not more than 1000 cells per square centimeter.

In one embodiment, the method also includes culturing the MSCs in the growth medium of the present invention.

The present invention also includes a method of increasing a population of rapidly self-renewing cells (RS cells) under in vitro culture conditions. The method includes plating the bone marrow stromal cells at an initial density of at least about 50 cells per square centimeter but not more than 1000 cells per square centimeter, incubating the cells for about four days, and harvesting the cells.

A method of detecting rapidly self-renewing cells (RS cells) in culture is also taught in the present invention. The method includes culturing marrow stromal cells for a period of time; sorting the cells into single-cell colonies using a flow cytometer; subjecting each cell colony to a forward and side scatter light assay; and comparing the forward scatter to side scatter results.

A method for minimizing rejection of bone marrow stromal cells cultured in vitro is taught in the present invention. The method includes culturing bone marrow stromal cells in growth medium that includes autologous serum. In one embodiment, the growth medium also includes epidermal growth factor, basic fibroblast growth factor, or combinations thereof.

The present invention also includes a method for isolating rapidly self-renewing cells (RS cells) from a population of bone marrow stromal cells. The method includes culturing a population of bone marrow stromal cells with a peptide derived from the LRP-6 binding domain of Dkk-1 (SEQ ID NO:10) wherein the peptide binds with an RS cell and detecting the peptide bound to the RS cell. Preferably, the peptide is selected from the group consisting of SEQ ID NO:12 and SEQ ID NO:15.

The present invention also includes a method for producing a sub-population of early progenitor MSCs in vitro. The method includes culturing the MSCs in serum-free medium for a period of time followed by a period of culturing in medium including serum. Preferably, the MSCs are incubated in serum free medium for about 3 weeks followed by a 5 day culture period in medium including serum.

### BRIEF DESCRIPTION OF THE DRAWINGS

FIG. 1 is a graph depicting initial plating density and expansion of MSCs. Passage 3 MSCs were plated on 60 cm² dishes at 10, 50, 100, and 1000 cells/cm². The cells were harvested and counted at 1 to 12 days. Data are expressed as mean ±SD (n=3).

FIG. 2, comprising FIGS. 2A-2D, is a set of graphs depicting the relationship between plating density and cell doubling times per day. Passage 3 MSCs were plated on 60 cm² dishes at 10 (FIG. 2A), 50 (FIG. 2B), 100 (FIG. 2C), and 1000 (FIG. 2D) cells/cm², harvested and counted at 1 to 12 days. Then cell doubling times per day were calculated.

FIG. 3 is a graph depicting the relationship between plating density and colony forming unit (CFU) efficiency. Passage 3 MSCs were plated on 60 cm² dishes at 10, 50, 100, and 1000 cells/cm² and cultured for 12 days. Values are number of colonies per 100 cells plated. Data are expressed as mean ±SD (n=3).

FIG. 4 is a graph depicting the relationship between initial plating density and total cell number. Passage 3 MSCs were plated on 60 cm² dishes at 10, 50, 100, and 1000 cells/cm². The cells were harvested and counted at 1 to 12 days. Total cell numbers per 60 cm² dish are shown. Data are expressed as mean ±SD (n=3).

FIG. 5 is a graph depicting plating density versus CFU efficiency, total yield, and total population doublings. CFU efficiency was measured after 12-day culture as stated in FIG. 3. Total yield per 60 cm² dish was measured after 12-day culture (see FIG. 4). Total population doublings were measured as 2ⁿ=fold increase, when n is equal to numbers of cell doublings.

FIG. 6, comprising FIGS. 6A and 6B, is a set of data showing the effect of initial cell density and time in culture on cell morphology. Passage 3 MSCs were plated at 10, 50, 100, and 1000 cells/cm². Photomicrographs of the cells were taken at 1 to 12 days. FIG. 6A is a set of images of representative pictures of MSCs plated at initial cell density of 50 cells/cm² at 1 to 12 days. FIG. 6B is a schematic diagram of MSC morphologies at 4 kinds of initial cell density at 1 to 12 days.

FIG. 7, comprising FIGS. 7A and 7B, indicates adipogenesis after a high density plating assay. FIG. 7A is a design scheme for adipogenesis after high density plating. FIG. 7B is an image of a set of photomicrographs of MSCs stained with oil red-o. The top two rows are low magnification 20×) and the bottom two rows are high magnification (150×).

FIG. 8, comprising FIGS. 8A-8D, depicts adipogenesis in a colony-forming assay. FIG. 8A is a design scheme for adipogenesis in a colony-forming assay. FIG. 8B is an image of adipocyte colonies stained with oil red-o (upper two panels) and crystal violet (lower two panels). FIG. 8C is a graph depicting the number of oil red-o positive and total colonies. FIG. 8D is a graph indicating the ratio of oil red-o positive colonies to the total number of colonies. Data are expressed as mean ±SD (n=3). Unpaired t-test was used for statistical analyses.

FIG. 9, comprising FIGS. 9A and 9B, depicts the effect of time in culture on chondrogenic potential of MSCs. FIG. 9A is a design scheme for the experiments. FIG. 9B is an image of a set of photomicrographs of pellets stained with toluidine blue sodium borate for proteoglycans.

FIG. 10, comprising FIGS. 10A and 10B, is a set of graphs illustrating the reproducibility of the single-cell colony forming unit (sc-CFU) assay. FIG. 10A illustrates the sc-CFU assay of MSCs and FIG. 10B illustrates the standard CFU assay of MSCs. (mean+/−SD, n=3 or 4).

FIG. 11, comprising FIGS. 11A, 11B, and 11C, is a set of scatter plots illustrating Annexin V exclusion. FIG. 11A is an assay of MSCs for forward scatter (FS-H) and side scatter (SC-H). FIG. 11B illustrates gating of Annexin V positive events (RI). FIG. 11C is the same sample as in FIG. 11B assayed after elimination of apoptotic cells by staining with Annexin V.

FIG. 12, comprising FIGS. 12A, 12B, 12C, and 12D, is a set of figures characteristics of clonal cells. FIG. 12A is a graph illustrating an sc-CFU assay of sorted cells. FIG. 12B represents the correlation between side scatter and aneuploidy as assayed by permeabilizing cells and staining with propidium iodide. FIG. 12C illustrates a microtiter plate of colonies from sc-CFU assay differentiated into osteoblasts (left) and a second microtiter plate stained with Crystal Violet (right). FIG. 12D illustrates that adipogenic and osteogenic lineages are not clonally restricted in non-senescent cells. On the left, osteogenic differentiation of a confluent culture stained with Alizarin Red S. A desiccated adipocyte is visible. Osteogenic differentiation of a single cell derived colony (Right) stained with (1st) Alizarin Red S and (2nd) Oil Red O. An adipocyte is in the process of taking up Oil Red O.

FIG. 13, comprising FIGS. 13A and 13B, is a set of graphs illustrating the differences between FS^{lo}/SS^{lo} cell and FS^{hi}/SS^{hi} cell expression of cell cycle related genes. Signal intensities are shown for 13 genes that showed the greatest difference between the two cell populations.

FIG. 14, comprising FIGS. 14A-14F, is a set graphs illustrating that large values of a derived flow meter are associated with a larger four-day fold change in cell number. FIG. 14A illustrates a FS and SS assay of passage 3 MSCs that were plated at 100 cells/cm² and incubated for 4 days. Vertical and horizontal lines are drawn on basis of calibration of instrument with microbeads. FIG. 14D illustrates a FS and SS assay of Passage 5 MSCs that were plated at 1,000 cells/cm² and incubated for 4 days. FIG. 14B is a bar graph of the derived flow parameter, and FIG. 14E is a bar graph of the derived fold change in cell number for cells from differing passages and initial plating densities. FIG. 14C is a standard curve for calibration of FS with microbeads of 7, 10, 15 and 20 microns. FIG. 14F is a bivariate plot depicting the relationship between fold change in cell number and a Flow Parameter defined by percent events in Region G divided by percent events in Region T shown in FIGS. 14A and 14D.

FIG. 15A is a graph depicting the growth of hMSCs after medium replacement containing various proportions of conditioned medium. Data are shown as the mean of three counts with error bars representing standard deviations.

FIG. 15B is an image depicting SDS-PAGE analysis of radiolabeled proteins secreted by hMSCs over time in culture. The radioactive bands at 180, 100 and 30 kDa are fibronectin (F), laminin (L) and Dkk-1 (asterisk), respectively.

FIG. 15C is an image depicting SDS-PAGE and silver staining of conditioned (C) and unconditioned (U) media.

FIG. 15D is an image depicting that the 30 kDa band from conditioned media shown in FIG. 15C was electroeluted, re-separated by SDS-PAGE and silver stained.

FIG. 15E is an image depicting SDS-PAGE and western blot analysis of medium from rapidly expanding hMSCs probed with a polyclonal antibody against the second cysteine rich domain of Dkk-1.

FIG. 15F depicts the recovery of Dkk-1 from conditioned medium by immunoaffinity chromatography.

FIG. 15G is an image depicting tryptic digestion and SELDI-TOF analysis of the 30 KDa band from FIG. 15C. The seven peptides corresponding to Dkk-1 within 0.5 Da are listed.

FIG. 15H represents the amino acid sequence of Dkk-1, and indicates the positions of the peptides listed in FIG. 15G in bold.

FIG. 16, comprising FIGS. 16A-16E, illustrates recombinant Dkk-1 enhances proliferation in hMSCs. FIG. 16A is an SDS-PAGE analysis of 5 micrograms Dkk-1 in reducing (R) and non-reducing (NR) conditions. The presence of monomeric (1), dimeric (2), trimeric (3) and multimeric forms are detectable via silver staining in the non-reduced form. FIG. 16B is a graph depicting the effect of 0.1 microgram per milliliter Dkk-1 on the proliferation curve of hMSCs. FIG. 16C is a graph depicting the effect of 0.01 microgram per milliliter recombinant Dkk-1 on the proliferation curve of hMSCs. FIG. 16D is a graph illustrating the number of visible colonies above 2 millimeters in diameter. FIG. 16E is a graph illustrating colonies that were measured and categorized based on diameter.

FIG. 17A is an image of the results of an RT-PCR assay of Dkk-1 and LRP-6 mRNA levels in hMSCs. The resulting fragments were analyzed by agarose gel electrophoresis followed by ethidium bromide staining.

FIG. 17B is a graph depicting hybridization ELISA analysis of PCR product Dkk-1 normalized against the appropriate GAPDH control. Results are expressed as a ratio of signal intensity versus GAPDH intensity. Error bars represent the standard deviation of the mean of 3 sets of data.

FIG. 17C is a graph depicting hybridization ELISA analysis of PCR product LRP-6 normalized against the appropriate GAPDH control. Results are expressed as a ratio of signal intensity versus GAPDH intensity. Error bars represent the standard deviation of the mean of 3 sets of data.

FIG. 17D is a graph depicting the analysis of beta-catenin levels and subcellular localization over time in culture by 4 to 12% SDS-PAGE and western blotting.

FIG. 18, comprising FIGS. 18A and 18B, is a graph key and a graph of the measurement of mRNA levels encoding members of the Wnt signaling pathways and related genes by microarray. FIG. 18A is the key to the graph (FIG. 18B) and indicates Genbank accession numbers. The signal intensities are plotted in arbitrary units.

FIG. 19, comprising FIGS. 19A and 19B, illustrates the effect of cell-cell contact and recombinant Dkk-1 on beta-catenin levels and distribution in hMSCs and HT 1080 cells. FIG. 19A is an image depicting visualization of beta-catenin levels by western blotting. (+) indicates treatment with recombinant Dkk-1 and (−) is control. FIG. 19B is an image of a set of photomicrographs illustrating hMSCs that were immunostained for beta-catenin and DAPI. FIGS. 19Bi and 19Bii Pare images of log phase cells. FIGS. 18Biii and 19Biv are images of stationary phase cells incubated in the presence or absence 0.1 microgram per milliliter recombinant Dkk-1. FIGS. 19Bv and 19Bvi are images of low power micrographs of confluent monolayers of hMSCs untreated or treated with Dkk-1. FIG. 19Bvii is an image of an isotype control.

FIGS. 20A and 20B are graphs comparing the cell cycle of hMSCs after 5 days in culture followed by addition of medium containing no FCS (FIG. 20A) or 20% (v/v) FCS (FIG. 20B). The relative proportions of cells in G1, S phase and G2 phase are indicated. Images of phase contrast micrographs are presented with each histogram illustrating cell density in each case.

FIG. 20C is an image depicting RT-PCR analysis of Dkk-1 transcription by hMSCs subjected to conditions described in FIGS. 20A and 20B.

FIG. 20D is a graph depicting hybridization ELISA analysis of the Dkk-1 PCR products normalized against the appropriate GAPDH control. Error bars represent the standard deviations of the mean of 3 sets of data.

FIG. 20E is an image depicting analysis of beta-catenin levels with or without 24 hours of serum starvation. Cellular beta-catenin levels were analyzed for both conditions tested using 4 to 12% SDS-PAGE and western blotting.

FIGS. 21A and 21B are graphs depicting the effect of anti-Dkk-1 polyclonal serum on proliferation of hMSCs from two donors after a change of medium. Data are expressed as a mean of 3 separate counts with error bars representing standard deviation.

FIG. 21C is an image depicting RT-PCR assay for levels of Dkk-1 mRNA in MG63 and SAOS osteosarcoma cell lines and two primitive choriocarcinomas.

FIG. 21D is a graph depicting the effect of anti Dkk-1 polyclonal antiserum on the proliferation of MG63 osteosarcoma cells.

FIG. 22, comprising FIGS. 22A and 22B, is an image of a set of photomicrographs depicting fluorescence microscopy results. FIG. 22A illustrates deconvolution microscopy of a human MSC from culture expanded in complete medium with 20% FITC-labeled FCS (fFCS). The cell contains internalized fFCS. FIG. 22B is an image depicting epifluorescence and phase microscopy of cultures expanded with 20% FCS (before) and transferred to AHS⁺ for 2 days (after).

FIG. 23 is a set of scatter plots depicting forward scatter and side scatter of cells plated at either 50 cells per cm² (low density) or 500 cells per cm² (high density), incubated in medium with 20% FCS for 4 days, and then transferred to AHS⁺ or FCS medium for an additional 48 hours.

FIG. 24, comprising FIGS. 24A and 24B, is a set of graphs illustrating hMSC yields initially plated at 50 cells/cm² (FIG. 24A) or 500 cells/cm² (FIG. 24B), incubated for 2 days in medium containing fFCS, and then for 2 days in serum-free medium, medium containing 20% FCS or AHS⁺. Data from two donors of hMSCs are shown (black and white bars).

FIG. 25 is a set of graphs illustrating fFCS per cell after expansion. FIG. 25A illustrates data collected with an initial plating of 50 cells/cm² and FIG. 25B illustrates data collected with an initial plating of 500 cells/cm².

FIG. 26 is a scatterplot of microarray data on expanded cells.

FIG. 27 illustrates the osteogenic and adipogenic differentiation of cells after expansion. Adipocytes were stained with Oil Red O and osteoblasts with Alizarin Red.

FIG. 28 lists amino acid sequence cys-2 peptide mapping of Dkk-1 (SEQ ID NO:10).

FIG. 29 lists 7 synthetic peptides (peptides A-G; SEQ ID NOS: 11-17) corresponding to cys-2 regions of the Dkk-1 protein (SEQ ID NO:10).

FIG. 30, comprising FIGS. 30A-30H, is an image of a set of photomicrographs depicting solid phase binding assays to MSCs using biotinylated peptides. The labeled peptides in FIGS. 30A-30G correspond to peptides A-G in FIG. 29. FIG. 30H is a control.

FIG. 31, comprising FIGS. 31A-31D, is an image of a set of phase-contrast micrographs depicting before (FIGS. 31A and 31B) and after (FIGS. 31C and 31D) recovery of serum-deprived MSCs in CCM. MSCs were recovered with 17% fetal calf serum. FIG. 31A is a control population of MSCs; FIG. 31B is 4 weeks serum deprived MSCS; FIG. 31C is one day post-recovery; FIG. 31D is 5 days post recovery.

FIG. 32, comprising FIGS. 32A, 32B, and 32C, is a graph and an image of a set of photomicrographs. FIG. 32A is a graph depicting the clonogenicity of serum derived and control MSCs. FIG. 32B is an image of a photomicrograph depicting adipocyte differentiation. FIG. 32C is an image of a photomicrograph depicting differentiation to mineralizing cells.

FIG. 33, comprising FIGS. 33A and 33B, is an image of a set of blots. FIG. 33A depicts telomere length in control and serum-deprived MSCs from three donors. HT1080, a human fibrosarcoma cell line, was used as a positive control. FIG. 33B is a Western blot detecting p53 and p21 in control and serum derived MSCs from three donors.

FIG. 34 is a schematic representation of how MSCs are prepared for microarray and RT-PCR. “SD” means serum deprived; “S” means with serum. “3wkSD” and “3wkS” means 3 weeks with our without serum. “+5dSDS” and “+5dS” means the “3wkSD” and “3wkS” samples incubated 5 days in medium with 17% fetal calf serum.

FIG. 35 is a photomicrograph of a gel depicting RT-PCR analysis of RNA obtained from the samples described in FIG. 34. The serum deprived MSCs demonstrated enhanced expression of early progenitor MSC genes. Row 1 is the OCT-4 gene; Row 2 is the ODC antizyme; Row 3 is HTERT; row 4 is beta-actin.

FIG. 36 is a schematic diagram of how data is analyzed from the microarrays.

FIG. 37 is a schematic of the hierarchical cluster analyses of 842 genes expressed in serum-deprived and control cells. The data on the graphs are presented as Day 0, 3wkSD, +5SDS, 3wkS, +5DSS (see FIG. 34 for legend).

FIG. 38, comprising FIGS. 38A-38J, is a set of graphs depicting prominent up/up and down/down dynamic response profiles (DRPs) for certain genes. The diamond line represents serum deprived cells and the square line represents control cells. FIG. 38A represents LOX, lysyl oxidase (Acc. No. NM_{—}002317); FIG. 38B represents GST, glutothione S transferase (AL527430); FIG. 38C represents SDNSF, neural stem cell derived neuronal survival protein (BE_{—}880828); FIG. 38D represents FGF2, fibroblast growth factor 2 (M27968); FIG. 38E represents KAP 1, keratin associated protein 1 (NM_{—}030967); FIG. 38F represents ATF5, activating transcription factor 5 (NM 012068); FIG. 38G represents ANP-1, angiopoietin-1 (U83508); FIG. 38H represents FGFR 2, fibroblast growth factor receptor-2 (NM_{—}022969); FIG. 38I represents SIX2, sine oculis homeobox homolog 2 (AF3332197); FIG. 38J represents HOXC6, homeobox C6 (NM004503).

FIG. 39 is a graph depicting the effect of peptide A on osteogenesis in a proliferative hMSC assay. Circles represent the vehicle control and the crosses represent the addition of 10 μg mL⁻¹ peptide A to the osteogenic medium.

FIG. 40 is a graph depicting the effect of the presence and absence of Dkk-1 on cellular recovery during bone morphogenic protein (BMP) and dexamethasone mediated osteogenesis of MSCs. The Y-axis represents the number of cells recovered per plate. A and B represents two separate donors.

FIG. 41 is a graph depicting the effect of the presence and absence of Dkk-1 on alkaline phosphatase (ALP) activity per cell during BMP and dexamethasone mediated osteogenesis of MSCs. The Y-axis represents micrograms of ALP recovered per plate. A and B represents two separate donors.

FIG. 42 is a graph depicting the effect of the presence (+) and absence (−) of Dkk-1 on total ALP activity during BMP and dexamethasone mediated osteogenesis of MSCs. Y-axis refers to micrograms of ALP recovered per plate. A and B refers to two separate donors.

FIG. 43 is a graph depicting the effect of the presence and absence of Dkk-1 on ALP activity per cell during BMP mediated osteogenesis of surviving MSCs that compensate for Dkk-1 induced apoptosis. The Y-axis represents micrograms of ALP recovered per plate

FIG. 44 is an image depicting the effect of lithium on osteogenic micromasses of MSCs.

FIG. 45 is a graph depicting osteogenesis of MSCs in the presence and absence of lithium as measured by Alizarin Red staining for calcium.

FIG. 46 is an image depicting osteogenesis of MSCs in the presence and absence of lithium as measured by RT-PCR for alkaline phosphatase message.

### DETAILED DESCRIPTION

The present invention includes methods of enhancing proliferation of MSCs. The present invention also encompasses methods and compositions for regulating the effects of Dkk-1 on the Wnt signaling pathway. The invention further provides a method of regulating the effects of Dkk-1 on cellular proliferation and differentiation. Methods and compositions for the treatment of osteolytic lesions in multiple myeloma. Another embodiment of the present invention includes methods and compositions for enhancing osteogenesis. A further embodiment of the present invention includes a method of detecting the presence of an osteolytic lesion in a mammal using the compositions of the present invention.

Definitions

The articles “a” and “an” are used herein to refer to one or to more than one (i.e., to at least one) of the grammatical objects of the article. By way of example, “an element” means one element or more than one element.

As used herein, “antagonist,” “Dkk-1 antagonist” and the like are meant to include any molecule that interacts with Dkk-1 and interferes with its function or blocks or neutralizes a relevant activity of Dkk-1, by whatever means. An antagonist may prevent the interaction between Dkk-1 and one or more of its receptors. Such an antagonist accomplishes this effect in various ways. For instance, the class of antagonists that “neutralizes” a Dkk-1 activity, binds to Dkk-1 with sufficient affinity and specificity so as to interfere with Dkk-1 function.

Included within this group of antagonists are, for example, antibodies directed against Dkk-1 or portions thereof reactive with Dkk-1, a Dkk-1 receptor or portions thereof reactive with Dkk-1, or any other ligands that bind to Dkk-1. The term antagonist also includes any agent that antagonizes at least one Dkk-1 receptor. Such antagonists may be in the form of an antibody, a protein or a peptide. In a preferred embodiment, the antagonist is a peptide corresponding to the LRP-6 binding site of Dkk-1, an antibody having the desirable properties of binding to Dkk-1 and preventing its interaction with a receptor. In a more preferred embodiment, the antagonist is peptide A (SEQ ID NO:11).

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer. As used in the present invention, the term “polypeptide” can refer to a sequence of as little as two amino acids linked by a peptide bond, or an unlimited number of amino acids linked by peptide bonds.

A “recombinant polypeptide” is one that is produced upon expression of a recombinant polynucleotide.

The term “protein” typically refers to large polypeptides.

The term “peptide” typically refers to short polypeptides.

A “mutant” polypeptide as used in the present application is one which has the identity of at least one amino acid altered when compared with the amino acid sequence of the naturally-occurring protein. Further, a mutant polypeptide may have at least one amino acid residue added or deleted to the amino acid sequence of the naturally-occurring protein.

Conventional notation is used herein to portray polypeptide sequences: the left-hand end of a polypeptide sequence is the amino-terminus; the right-hand end of a polypeptide sequence is the carboxyl-terminus. As used herein, the term “fragment” as applied to a polypeptide, may ordinarily be at least about 20 amino acids in length, preferably, at least about 30 amino acids, more typically, from about 40 to about 50 amino acids, preferably, at least about 50 to about 80 amino acids, even more preferably, at least about 80 amino acids to about 90 amino acids, yet even more preferably, at least about 90 to about 100, even more preferably, at least about 100 amino acids to about 120 amino acids, and most preferably, the amino acid fragment will be greater than about 123 amino acids in length.

As used herein, to “alleviate” a disease means reducing the severity of one or more symptoms of the disease.

A “disease” is a state of health of an animal wherein the animal cannot maintain homeostasis, and wherein if the disease is not ameliorated, then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

To “treat” a disease as the term is used herein refers to a situation where the severity of a symptom of a disease or the frequency with which any symptom or sign of the disease is experienced by a patient, is reduced.

“Osteolytic lesion,” as used herein, means a common skeletal manifestation in a patient including but not limited to bone degradation, the osteoclast accumulation on bone-resorbing surfaces in myeloma adjacent to a collection of myeloma cells, and/or the increase in osteoclastic bone resorption in myeloma that is associated with a marked impairment in osteoblast function.

By the term “effective amount” of an Dkk-1 antagonist, as the term is used herein, means an amount of an Dkk-1 antagonist that produces a detectable effect on Dkk-1 function and/or biological activity or characteristic. Such effect can be assessed using a variety of assays either disclosed herein, known in the art, or to be developed. A characteristic and/or biological activity that is assessed includes, but is not limited to, the ability of Dkk-1 to modulate the Wnt pathway. As used herein, the term “modulating Dkk-1” is meant to refer to the change in the effects of Dkk-1.

Instructional material,” as that term is used herein, includes a publication, a recording, a diagram, or any other medium of expression which can be used to communicate the usefulness of the nucleic acid, peptide, and/or compound of the invention in the kit for effecting alleviating or treating the various diseases or disorders recited herein. Optionally, or alternately, the instructional material may describe one or more methods of alleviating the diseases or disorders in a cell or a tissue of a mammal. The instructional material of the kit may, for example, be affixed to a container that contains the nucleic acid, peptide, and/or compound of the invention or be shipped together with a container which contains the nucleic acid, peptide, and/or compound. Alternatively, the instructional material may be shipped separately from the container with the intention that the recipient uses the instructional material and the compound cooperatively.

A “receptor” is a compound that specifically binds with a ligand.

By the term “specifically binds,” as used herein, is meant a compound, e.g., a protein, a nucleic acid, an antibody, and the like, which recognizes and binds a specific molecule, but does not substantially recognize or bind other molecules in a sample. For instance, an antibody or a peptide inhibitor that recognizes and binds a cognate ligand (i.e., an anti-Dkk-1 antibody that binds to Dkk-1) in a sample, but does not substantially recognize or bind other molecules in the sample.

The term “about” will be understood by persons of ordinary skill in the art and will vary to some extent on the context in which it is used. If there are uses of the term which are not clear to persons of ordinary skill in the art given the context in which it is used, “about” shall mean up to plus or minus 10% of the particular value.

As used herein, the term “bone marrow stromal cells,” “stromal cells” or “MSCs” are used interchangeably and refer to the small fraction of cells in bone marrow which can serve as stem cell-like precursors to osteocytes, chondrocytes, monocytes, and adipocytes, and which are isolated from bone marrow by their ability to adhere to plastic dishes. Marrow stromal cells may be derived from any animal. In some embodiments, stromal cells are derived from primates, preferably humans.

As used herein, the term “enhancing multipotentiality” of bone marrow stromal cells is meant to refer to an increase in production of multipotent bone marrow stromal cells in a bone marrow stromal cell culture.

As used herein, the term “growth medium” is meant to refer to a culture medium that promotes growth of cells. A growth medium will generally contain animal serum.

As used herein, the term “exogenous” refers to any material introduced from or produced outside an organism, cell, or system.

As used herein, the term “autologous” is meant to refer to any material derived from the same individual to which it is re-introduced.

Description

The invention relates to compositions and methods for modulating Dkk-1 activity, as well as compositions and methods of treating an osteolytic lesion in a mammal. As discussed elsewhere herein, an osteolytic lesion may be caused by cancers such as, but not limited to lung, breast, prostate cancer and multiple myeloma. In addition, the invention relates to compositions and methods for modulating proliferation and osteogenesis of a cell in a mammal.

Until the present invention, technical obstacles had impeded the modulation of Dkk-1 and biological functions associated with Dkk-1. The data disclosed herein identify novel methods and compositions for the successful modulation of Dkk-1 activity. Further, the invention relates to novel methods for detecting the presence of a disease state wherein Dkk-1 is deregulated for example in an osteolytic lesion in a mammal.

I. Isolated Nucleic Acids

The present invention includes an isolated nucleic acid encoding a Dkk-1 antagonist, or a biologically active fragment thereof. The skilled artisan, based upon the disclosure provided herein, would understand that the nucleic acids of the invention are useful for production of the peptide of interest. The nucleic acids of the invention are not limited to products of any of the specific exemplary processes listed herein. Preferably, the nucleic acids encoding the polypeptides of the present invention are derived from the amino acid sequence of the LRP-6 binding domain of Dkk-1. The sequences provided below are representative amino acid and corresponding nucleic acid sequence of the LRP-6 binding domain of Dkk-1.

(SEQ ID NO:10)

GNDHSTLDGYSRRTTLSSKMYHTKGQEGSVCLRSSDCASGLCCARHFWSK
ICKPVLKEGQVCTKHRRKGSHGLEIFQRCYCGEGLSCRIQKDHHQASNSS
RLHTCQRH:

(SEQ ID NO:46)

ggtaatg atcatagcac cttggatggg tattccagaa
gaaccacctt gtcttcaaaa atgtatcaca ccaaaggaca
agaaggttct gtttgtctcc ggtcatcaga ctgtgcctca
ggattgtgtt gtgctagaca cttctggtcc aagatctgta
aacctgtcct gaaagaaggt caagtgtgta ccaagcatag
gagaaaaggc tctcatggac tagaaatatt ccagcgttgt
tactgtggag aaggtctgtc ttgccggata cagaaagatc
accatcaagc cagtaattct tctaggcttc acacttgtca
gagacac

Selected cysteines in the following peptides were substituted with serines to facilitate production of the peptides. These substitutions are indicated by the lowercase “s” in the sequence. The synthesized peptide sequences were as follows (also depicted in FIG. 29):

GNDHSTLDGYSRRTTLSSKM;
(Peptide A; SEQ ID NO:11)
ggtaatgatcatagcaccttggatgggtattccagaagaaccaccttgtc
ttcaaaaatg
(SEQ ID NO:47)
LSSKMYHTKGQEGSVCLRSS;
(Peptide B; SEQ ID NO:12)
ttgtcttcaaaaatgtatcacaccaaaggacaagaaggttctgtttgtct
ccggtcatca
(SEQ ID NO:48)
sLRSSDCASGLCCARHFWSK;
(Peptide C; SEQ ID NO:13)
nnnctccggtcatcagactgtgcctcaggattgtgttgtgctagacactt
ctggtccaag
nnn could be tct, tcc, tca, tcg or agt
(SEQ ID NO:49)
FWSKICKPVLKEGQVCTKHR;
(Peptide D; SEQ ID NO:14)
ttctggtccaagatctgtaaacctgtcctgaaagaaggtcaagtgtgtac
caagcatagg
(SEQ ID NO:50)
sTKHRRKGSHGLEIFQRCYs;
(Peptide E; SEQ ID NO:15)
nnnaccaagcataggagaaaaggctctcatggactagaaatattccagcg
ttgttacnnn
nnn could be tct, tcc, tca, tcg or agt
(SEQ ID NO:51)
QRCYsGEGLSCRIQKDHHQA;
(Peptide F; SEQ ID NO:16)
cagcgttgttacnnnggagaaggtctgtcttgccggatacagaaagatca
ccatcaagcc
nnn could be tct, tcc, tca, tcg or agt
(SEQ ID NO:52)
DHHQASNSSRLHTCQRH;
(Peptide G; SEQ ID NO:17)
gatcaccatcaagccagtaattcttctaggcttcacacttgtcagagaca
c
(SEQ ID NO:53)

The isolated nucleic acid of the invention should be construed to include an RNA or a DNA sequence encoding a Dkk-1 antagonist of the invention, and any modified forms thereof, including chemical modifications of the DNA or RNA. Chemical modifications of nucleotides may also be used to enhance the efficiency with which a nucleotide sequence is taken up by a cell or the efficiency with which it is expressed in a cell. Any and all combinations of modifications of the nucleotide sequences are contemplated in the present invention.

The present invention should not be construed as being limited solely to the nucleic and amino acid sequences disclosed herein. Once armed with the present invention, it is readily apparent to one skilled in the art that other nucleic acids encoding antagonists of Dkk-1 can be identified, such as, but not limited to, other nucleic acids encoding human Dkk-1 antagonists, as well as nucleic acids present in other species of mammals (e.g., ape, gibbon, bovine, ovine, equine, porcine, canine, feline, and the like). These additional sequences can be obtained by following the procedures described herein in the experimental details section and procedures that are well-known in the art, or to be developed in the future.

Further, any number of procedures may be used for the generation of mutant, derivative or variant forms of Dkk-1 antagonists using recombinant DNA methodology well known in the art such as, for example, that described in Sambrook et al. (1989, Molecular Cloning: A Laboratory Manual, Cold Spring Harbor Laboratory Press, New York) and Ausubel et al. (1997, Current Protocols in Molecular Biology, Green & Wiley, New York).

Procedures for the introduction of amino acid changes in a protein or polypeptide by altering the DNA sequence encoding the polypeptide are well known in the art and are also described in Sambrook et al. (1989, supra); Ausubel et al. (1997, supra).

The invention includes a nucleic acid encoding a mammalian Dkk-1 antagonist, wherein a nucleic acid encoding a tag polypeptide is covalently linked thereto. That is, the invention encompasses a chimeric nucleic acid wherein the nucleic acid sequences encoding a tag polypeptide is covalently linked to the nucleic acid encoding at least one Dkk-1 antagonist, or biologically active fragment thereof. Such tag polypeptides are well known in the art and include, for instance, green fluorescent protein (GFP), an influenza virus hemagglutinin tag polypeptide, myc, myc-pyruvate kinase (myc-PK), His₆, maltose binding protein (MBP), a FLAG tag polypeptide, and a glutathione-S-transferase (GST) tag polypeptide. However, the invention should in no way be construed to be limited to the nucleic acids encoding the above-listed tag polypeptides. Rather, any nucleic acid sequence encoding a polypeptide which may function in a manner substantially similar to these tag polypeptides should be construed to be included in the present invention.

The nucleic acid comprising a nucleic acid encoding a tag polypeptide can be used to localize a Dkk-1 antagonist, or a biologically active fragment thereof, within a cell, a tissue (e.g., a blood vessel, bone, and the like), and/or a whole organism (e.g., a human, and the like), and to study the role(s) of an Dkk-1 antagonist in a cell. Further, addition of a tag polypeptide facilitates isolation and purification of the “tagged” protein such that the proteins of the invention can be produced and purified readily.

II. Isolated Polypeptides

The invention also includes an isolated polypeptide comprising a mammalian a Dkk-1 antagonist, or a biologically active fragment thereof. One skilled in the art would appreciate, base upon the disclosure provided herein, that a Dkk-1 antagonist can be derived from the LRP-6 binding site of Dkk-1.

The invention encompasses a biologically active fragment of a Dkk-1 antagonist of the invention. That is, the skilled artisan would appreciate, based upon the disclosure provided herein, that a fragment of the Dkk-1 antagonist of the invention can be used in the methods of the invention.

The present invention also provides for analogs of proteins or peptides which comprise an Dkk-1 antagonist, or biologically active fragment thereof, as disclosed herein. Analogs may differ from naturally occurring proteins or peptides by conservative amino acid sequence differences or by modifications which do not affect sequence, or by both. For example, conservative amino acid changes may be made, which although they alter the primary sequence of the protein or peptide, do not normally alter its function. Conservative amino acid substitutions typically include substitutions within the following groups:

glycine, alanine;

valine, isoleucine, leucine;

aspartic acid, glutamic acid;

asparagine, glutamine;

serine, threonine;

lysine, arginine;

phenylalanine, tyrosine.

Modifications (which do not normally alter primary sequence) include in vivo, or in vitro, chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

Preferably, the polypeptides of the present invention are described elsewhere herein as set forth in SEQ ID Nos:11-17. More preferably, the Dkk-1 antagonist is Peptide A (GNDHSTLDGYSRRTTLSSKM (SEQ ID NO:11).

The present invention should also be construed to encompass “mutants,” “derivatives,” and “variants” of the peptides of the invention (or of the DNA encoding the same) which mutants, derivatives and variants are Dkk-1 antagonist, or biologically active fragment thereof, are altered in one or more amino acids (or, when referring to the nucleotide sequence encoding the same, are altered in one or more base pairs) such that the resulting peptide (or DNA) is not identical to the sequences recited herein, but has the same biological property as the peptides disclosed herein, in that the peptide has biological/biochemical properties of the Dkk-1 antagonist, or biologically active fragment thereof of the present invention.

Further, the invention should be construed to include naturally occurring variants or recombinantly derived mutants of Dkk-1 antagonist, or biologically active fragment thereof, sequences, which variants or mutants render the protein encoded thereby either more, less, or just as biologically active as full-length Dkk-1.

Further, the nucleic and amino acids of the invention can be used diagnostically, either by assessing the level of gene expression or protein expression, to assess severity and prognosis of a disease, disorder or condition mediated by Dkk-1. The nucleic acids and proteins of the invention are also useful in the development of assays to assess the efficacy of a treatment for treating, ameliorating, or both, such disease, and the like.

III. Vectors

In other related aspects, the invention includes an isolated nucleic acid encoding a Dkk-1 antagonist, or biologically active fragment thereof, operably linked to a nucleic acid comprising a promoter/regulatory sequence such that the nucleic acid is preferably capable of directing expression of the protein encoded by the nucleic acid. Thus, the invention encompasses expression vectors and methods for the introduction of exogenous DNA into cells with concomitant expression of the exogenous DNA in the cells such as those described, for example, in Sambrook et al. (1989, supra), and Ausubel et al. (1997, supra).

Expression of a Dkk-1 antagonist, or biologically active fragment thereof, either alone or fused to a detectable tag polypeptide, in cells which either do not normally express the Dkk-1 antagonist, or biologically active fragment thereof, fused with a tag polypeptide, may be accomplished by generating a plasmid, viral, or other type of vector comprising the desired nucleic acid operably linked to a promoter/regulatory sequence which serves to drive expression of the protein, with or without tag, in cells in which the vector is introduced. Many promoter/regulatory sequences useful for driving constitutive expression of a gene are available in the art and include, but are not limited to, for example, the cytomegalovirus immediate early promoter enhancer sequence, the SV40 early promoter, both of which were used in the experiments disclosed herein, as well as the Rous sarcoma virus promoter, and the like.

Moreover, inducible and tissue specific expression of the nucleic acid encoding a Dkk-1 antagonist, or biologically active fragment thereof, may be accomplished by placing the nucleic acid encoding a Dkk-1 antagonist, or biologically active fragment thereof, with or without a tag, under the control of an inducible or tissue specific promoter/regulatory sequence. Examples of tissue specific or inducible promoter/regulatory sequences which are useful for his purpose include, but are not limited to the MMTV LTR inducible promoter, and the SV40 late enhancer/promoter. In addition, promoters which are well known in the art which are induced in response to inducing agents such as metals, glucocorticoids, and the like, are also contemplated in the invention. Thus, it will be appreciated that the invention includes the use of any promoter/regulatory sequence, which is either known or unknown, and which is capable of driving expression of the desired protein operably linked thereto.

The invention thus includes a vector comprising an isolated nucleic acid encoding a Dkk-1 antagonist, or biologically active fragment thereof. The incorporation of a desired nucleic acid into a vector and the choice of vectors is well-known in the art as described in, for example, Sambrook et al., supra, and Ausubel et al., supra.

The invention also includes cells, viruses, proviruses, and the like, containing such vectors. Methods for producing cells comprising vectors and/or exogenous nucleic acids are well-known in the art. See, e.g., Sambrook et al., supra; Ausubel et al., supra.

IV. Antibodies

The invention also encompasses monoclonal, synthetic antibodies, and the like. One skilled in the art would understand, based upon the disclosure provided herein, that the crucial feature of the Dkk-1 antagonist of the invention is that the Dkk-1 antagonist inhibits the Dkk-1/LRP-6 complex. That is, an anti-Dkk-1 antibody of the present invention abrogates the association of Dkk-1 with a Dkk-1 receptor for example the lipoprotein-related receptor protein-6 (LRP-6).

The generation of polyclonal antibodies is accomplished by inoculating the desired animal with the antigen and isolating antibodies which specifically bind the antigen therefrom using standard antibody production methods such as those described in, for example, Harlow et al. (1988, In: Antibodies, A Laboratory Manual, Cold Spring Harbor, N.Y.). Such techniques include immunizing an animal with a chimeric protein comprising a portion of another protein such as a maltose binding protein or glutathione (GST) tag polypeptide portion, and/or a moiety such that the Dkk-1 or fragments thereof portion is rendered immunogenic (e.g., Dkk-1 conjugated with keyhole limpet hemocyanin, KLH) and a portion comprising the respective rodent and/or human Dkk-1 amino acid residues. The chimeric proteins are produced by cloning the appropriate nucleic acids encoding Dkk-1 or fragments thereof (e.g., SEQ ID NO:11 into a plasmid vector suitable for this purpose, such as but not limited to, pMAL-2 or PCMX. Other methods of producing antibodies that specifically bind Dkk-1 or fragments thereof are detailed in Matthews et al. (2000, J. Biol. Chem. 275:22695-22703).

However, the invention should not be construed as being limited solely to polyclonal antibodies that bind a full-length Dkk-1. Rather, the invention should be construed to include other antibodies, as that term is defined elsewhere herein, to mammalian Dkk-1, or portions thereof. Further, the present invention should be construed to encompass antibodies that, among other, bind to Dkk-1 or fragments thereof and are able to bind Dkk-1 or fragments thereof present on Western blots, in immunohistochemical staining of tissues thereby localizing Dkk-1 in the tissues, and in immunofluorescence microscopy of a cell transiently or stably transfected with a nucleic acid encoding at least a portion of Dkk-1.

One skilled in the art would appreciate, based upon the disclosure provided herein, that the antibody can specifically bind with any portion of the protein and the full-length protein can be used to generate antibodies specific therefor. However, the present invention is not limited to using the full-length protein as an immunogen. Rather, the present invention includes using an immunogenic portion of the protein to produce an antibody that specifically binds with mammalian Dkk-1. That is, the invention includes immunizing an animal using an immunogenic portion, or antigenic determinant, of the Dkk-1 protein, for example, the epitope comprising the LRP-6 binding site of Dkk-1.

The antibodies can be produced by immunizing an animal such as, but not limited to, a rabbit or a mouse, with a Dkk-1 protein, or a portion thereof, or by immunizing an animal using a protein comprising at least a portion of Dkk-1, or a fusion protein including a tag polypeptide portion comprising, for example, a maltose binding protein tag polypeptide portion, covalently linked with a portion comprising the appropriate Dkk-1 amino acid residues. One skilled in the art would appreciate, based upon the disclosure provided herein, that smaller fragments of these proteins can also be used to produce antibodies that specifically bind Dkk-1.

One skilled in the art would appreciate, based upon the disclosure provided herein, that various portions of an isolated Dkk-1 polypeptide can be used to generate antibodies to either epitopes comprising the LRP-6 binding site of Dkk-1. Once armed with the sequence of Dkk-1 and the detailed analysis of the LRP-6 binding site of Dkk-1, the skilled artisan would understand, based upon the disclosure provided herein, how to obtain antibodies specific for the various portions of a mammalian Dkk-1 polypeptide using methods well-known in the art or to be developed.

Therefore, the skilled artisan would appreciate, based upon the disclosure provided herein, that the present invention encompasses antibodies that neutralize and/or inhibit Dkk-1 activity, which antibodies can recognize Dkk-1 or Dkk-1 fragments thereof.

The invention should not be construed as being limited solely to the antibodies disclosed herein or to any particular immunogenic portion of the proteins of the invention. Rather, the invention should be construed to include other antibodies, as that term is defined elsewhere herein, to Dkk-1, or portions thereof, or to proteins sharing at least about 50% homology with Dkk-1. Preferably, the polypeptide is about 60% homologous, more preferably, about 70% homologous, even more preferably, about 80% homologous, preferably, about 90% homologous, more preferably, about 95% homologous, even more preferably, about 99% homologous, and most preferably, about 99.9% homologous to Dkk-1.

One skilled in the art would appreciate, based upon the disclosure provided herein, that the antibodies can be used to localize the relevant protein in a cell and to study the role(s) of the antigen recognized thereby in cell processes. Moreover, the antibodies can be used to detect and or measure the amount of protein present in a biological sample using well-known methods such as, but not limited to, Western blotting and enzyme-linked immunosorbent assay (ELISA). Moreover, the antibodies can be used to immunoprecipitate and/or immuno-affinity purify their cognate antigen using methods well-known in the art.

In addition, the antibody can be used to decrease the level of Dkk-1 or Dkk-1 fragments thereof in a cell thereby inhibiting the effect(s) of Dkk-1 in a cell. Thus, by administering the antibody to a cell or to the tissues of a mammal or to the mammal itself, the required Dkk-1 receptor/ligand interactions are therefore inhibited such that the effect of Dkk-1 on the Wnt signaling pathway is also inhibited. One skilled in the art would understand that inhibiting Dkk-1 activity with an anti-Dkk-1 antibody can include, but is not limited to, treat an osteolytic lesion in multiple myeloma, enhance osteogenesis, modulate cellular proliferation, and the like.

One skilled in the art would appreciate, based upon the disclosure provided herein, that the invention encompasses administering an antibody that specifically binds with Dkk-1 orally, parenterally, intraventricularly, intrathecally. intraparenchymally or by multiple routes, to inhibit Dkk-1 activity.

The invention encompasses polyclonal, monoclonal, synthetic antibodies, and the like. One skilled in the art would understand, based upon the disclosure provided herein, that the crucial feature of the antibody of the invention is that the antibody bind specifically with Dkk-1. That is, the antibody of the invention recognizes Dkk-1, or a fragment thereof (e.g., an immunogenic portion or antigenic determinant thereof), on Western blots, in immunostaining of cells, and immunoprecipitates Dkk-1 using standard methods well-known in the art.

Monoclonal antibodies directed against full length or peptide fragments of a protein or peptide may be prepared using any well known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art, and is described, for example, in Wright et al. (1992, Critical Rev. Immunol. 12:125-168), and the references cited therein.

Further, the antibody of the invention may be “humanized” using the technology described in, for example, Wright et al. (1992, Critical Rev. Immunol. 12:125-168), and in the references cited therein, and in Gu et al. (1997, Thrombosis and Hematocyst 77:755-759). The present invention also includes the use of humanized antibodies specifically reactive with epitopes of Dkk-1. Such antibodies are capable of specifically binding Dkk-1, or a fragment thereof. The humanized antibodies of the invention have a human framework and have one or more complementarity determining regions (CDRs) from an antibody, typically, but not limited to a mouse antibody, specifically reactive with Dkk-1, or a fragment thereof. Thus, for example, humanized antibodies to Dkk-1 are useful in the treatment of an osteolytic lesion in multiple myeloma. The humanized antibodies of the present invention can also be used to enhance osteogenesis.

When the antibody used in the invention is humanized, the antibody may be generated as described in Queen, et al. (U.S. Pat. No. 6,180,370), Wright et al., (1992, Critical Rev. Immunol. 12:125-168) and in the references cited therein, or in Gu et al. (1997, Thrombosis and Hematocyst 77(4):755-759). The method disclosed in Queen et al. is directed in part toward designing humanized immunoglobulins that are produced by expressing recombinant DNA segments encoding the heavy and light chain complementarity determining regions (CDRs) from a donor immunoglobulin capable of binding to a desired antigen, such as Dkk-1, attached to DNA segments encoding acceptor human framework regions. Generally speaking, the invention in the Queen patent has applicability toward the design of substantially any humanized immunoglobulin. Queen explains that the DNA segments will typically include an expression control DNA sequence operably linked to the humanized immunoglobulin coding sequences, including naturally-associated or heterologous promoter regions. The expression control sequences can be eukaryotic promoter systems in vectors capable of transforming or transfecting eukaryotic host cells or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, (1979), which is incorporated herein by reference).

Human constant region (CDR) DNA sequences from a variety of human cells can be isolated in accordance with well known procedures. Preferably, the human constant region DNA sequences are isolated from immortalized B-cells as described in WO87/02671, which is herein incorporated by reference. CDRs useful in producing the antibodies of the present invention may be similarly derived from DNA encoding monoclonal antibodies capable of binding to Dkk-1. Such humanized antibodies may be generated using well known methods in any convenient mammalian source capable of producing antibodies, including, but not limited to, mice, rats, rabbits, or other vertebrates. Suitable cells for constant region and framework DNA sequences and host cells in which the antibodies are expressed and secreted, can be obtained from a number of sources, for example, American Type Culture Collection, Manassas, Va.

In addition to the humanized antibodies discussed above, other modifications to native antibody sequences can be readily designed and manufactured utilizing various recombinant DNA techniques well known to those skilled in the art. Moreover, a variety of different human framework regions may be used singly or in combination as a basis for humanizing antibodies directed to Dkk-1. In general, modifications of genes may be readily accomplished using a variety of well-known techniques, such as site-directed mutagenesis (Gillman and Smith, Gene, 8:81-97 (1979); Roberts et al., 1987, Nature, 328:731-734).

Alternatively, a phage antibody library may be generated. To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (1989, Molecular Cloning: A Laboratory Manual, Cold Spring Harbor Laboratory, New York).

Bacteriophage which encode the desired antibody, may be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art and are described for example, in Wright et al. (992, Critical Rev. Immunol. 12:125-168).

Processes such as those described above, have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the invention should not be construed to be limited solely to the generation of phage encoding Fab antibodies. Rather, phage which encode single chain antibodies (scFv/phage antibody libraries) are also included in the invention. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al. (1991, J. Mol. Biol. 222:581-597). Panning of phage so generated for the isolation of a desired antibody is conducted in a manner similar to that described for phage libraries comprising Fab DNA.

The invention should also be construed to include synthetic phage display libraries in which the heavy and light chain variable regions may be synthesized such that they include nearly all possible specificities (Barbas, 1995, Nature Medicine 1:837-839; de Kruif et al. 1995, J. Mol. Biol. 248:97-105).

V. Compositions

The invention includes a composition comprising a Dkk-1 inhibitor or a biologically active fragment thereof. As discussed elsewhere herein, the Dkk-1 inhibitor includes but is not limited to a peptide corresponding to the LRP-6 binding site of Dkk-1 and the nucleic acid sequence encoding the peptide. For example, the Dkk-1 antagonist of the present invention include the peptides designated by SEQ ID Nos:11-17. Preferably, the Dkk-1 antagonist is the peptide of SEQ ID NO:11.

The composition of the present invention also includes an antibody that specifically binds to Dkk-1. More preferably, the composition of the present invention comprises a pharmaceutically acceptable carrier.

The compositions can be used to administer an effective amount of a Dkk-1 antagonist, or a biologically active fragment thereof, to a cell, a tissue, or an animal. The compositions are useful to treat a disease, disorder or condition mediated by Dkk-1. That is, where a disease, disorder or condition (e.g., osteolyic lesion, among others) in an animal is mediated by, or associated with, Dkk-1, the composition can be used to modulate Dkk-1.

For administration to the mammal, a polypeptide, or a nucleic acid encoding it or a portion thereof, can be suspended in any pharmaceutically acceptable carrier, for example, HEPES buffered saline at a pH of about 7.8.

Another aspect of the present invention relates to the discovery that lithium and/or other inhibitors of GSK3β can be used to inhibiting the effects of Dkk-1. That is, one skilled in the art when armed with the present application would recognize that lithium and/or other inhibitors of GSK3β would inhibit the effects of Dkk-1 on the Wnt signaling pathway and prevent the phosphorylation of β-catenin.

The skilled artisan would understand that the effective amount varies and can be readily determined based on a number of factors such as the disease or condition being treated, the age and health and physical condition of the mammal being treated, the severity of the disease, the particular compound being administered, and the like. Generally, the effective amount will be between about 0.1 mg/kg to about 100 mg/kg, more preferably from about 1 mg/kg and 25 mg/kg. The compound (e.g., an Dkk-1 antagonist, or biologically active fragment thereof, a peptide inhibitor, and the like) can be administered through intravenous injection, including, among other things, a bolus injection. However, the invention is not limited to this method of administration.

Other pharmaceutically acceptable carriers which are useful include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides.

Pharmaceutical compositions that are useful in the methods of the invention may be administered, prepared, packaged, and/or sold in formulations suitable for oral, rectal, vaginal, parenteral, topical, pulmonary, intranasal, buccal, ophthalmic, or another route of administration. Other contemplated formulations include projected nanoparticles, liposomal preparations, resealed erythrocytes containing the active ingredient, and immunologically-based formulations.

The compositions of the invention may be administered via numerous routes, including, but not limited to, oral, rectal, vaginal, parenteral, topical, pulmonary, intranasal, buccal, or ophthalmic administration routes. The route(s) of administration will be readily apparent to the skilled artisan and will depend upon any number of factors including the type and severity of the disease being treated, the type and age of the veterinary or human patient being treated, and the like.

Pharmaceutical compositions that are useful in the methods of the invention may be administered systemically in oral solid formulations, ophthalmic, suppository, aerosol, topical or other similar formulations. In addition to the compound such as heparan sulfate, or a biological equivalent thereof, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer a Dkk-1 antagonist, or a biologically active portion thereof, and/or a nucleic acid encoding the same, according to the methods of the invention.

Compounds which are identified using any of the methods described herein may be formulated and administered to a mammal for treatment of osteolytic lesion and the like, are now described.

The invention encompasses the preparation and use of pharmaceutical compositions comprising a compound useful for treatment of treatment of osteolytic lesion, and the like, as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “pharmaceutically acceptable carrier” means a chemical composition with which the active ingredient may be combined and which, following the combination, can be used to administer the active ingredient to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

Pharmaceutical compositions that are useful in the methods of the invention may be prepared, packaged, or sold in formulations suitable for oral, rectal, vaginal, parenteral, topical, pulmonary, intranasal, buccal, ophthalmic, intrathecal or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken, i.e., by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other ophthalmalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

Typically, dosages of the compound of the invention which may be administered to an animal, preferably a human, will vary depending upon any number of factors, including but not limited to, the type of animal and type of disease state being treated, the age of the animal and the route of administration.

The compound can be administered to an animal as frequently as several times daily, or it may be administered less frequently, such as once a day, once a week, once every two weeks, once a month, or even lees frequently, such as once every several months or even once a year or less. The frequency of the dose will be readily apparent to the skilled artisan and will depend upon any number of factors, such as, but not limited to, the type and severity of the disease being treated, the type and age of the animal, etc.

VI. Methods

A. Methods of Identifying a Useful Compound for Modulating Dkk-1 Activity.

The invention encompasses a method of identifying a Dkk-1 antagonist that is capable of antagonizing Dkk-1. Another aspect of the present encompasses identifying a composition that can inhibit the effect of Dkk-1 on the Wnt signaling pathway. This later method provides a powerful tool for a composition having an inhibitory effect of Dkk-1 on the Wnt signaling and/or identifying a Dkk-1 antagonist that can modulate Dkk-1, wherein the modulation of Dkk-1 can alleviate a disease, disorder or condition in a mammal. Accordingly, a method is provided for identifying a Dkk-1 antagonist that is capable of antagonizing the effect of Dkk-1 on the Wnt signaling. An example of a method for identifying a Dkk-1 antagonist comprises assessing the osteoblastic differentiation potential of a MSC in the presence or absence of a Dkk-1 antagonist. The effect of the Dkk-1 antagonist on the differentiation of a MSC into a osteoblast can be assessed by analyzing positive staining with Alizarin Red S and the presence of mineralization as markers for osteoblast differentiation. While not wishing to be bound to any particular theory, in this assay, a Dkk-1 antagonist exhibits an increase rate of osteogenic differentiation of MSCs when compared with a control compound or a compound that does not antagonize Dkk-1.

Another method of identifying a Dkk-1 antagonist is to assess the ability of the compound to effect the downstream components of the Wnt signaling pathway, for example, but not limited to the level of cytosolic β-catenin. As discussed elsewhere herein, Dkk-1 has been demonstrated to inhibit the Wnt signaling pathway, and therefore a potential Dkk-1 antagonist would reverse the inhibitory activity of Dkk-1 on the Wnt signaling pathway and the downstream targets of the Wnt signaling pathway. In essence, a Dkk-1 antagonist would activate the Wnt signaling pathway and the down stream targets. That is, one skilled in the art based upon the present disclosure would appreciate that any method known in the art to measure the phosphorylation level of β-catenin and the protein level of β-catenin can be used to assess the potential of a Dkk-1 antagonist to inhibit Dkk-1 function on the Wnt signaling pathway. While not wishing to be bound to any particular theory, the downstream effects of having the Wnt signaling pathway activated include the phosphorylation and inactivation of GSK3β, resulting in inhibition of phosphorylation and degradation of β-catenin. Thus, methods known in the art such as but not limited to Western blot analysis using antibodies to β-catenin and phospho-specific antibodies to β-catenin and/or GSK3β can be used to assess the activity of the Dkk-1 antagonist. The invention should not be construed to be limited to any of the general assay methods disclosed herein and measurement of Dkk-1 activity or effects on Wnt signaling can be accomplished using any methods known or heretofore unknown in the art.

Further, the Dkk-1 antagonist identified by this method, as disclosed elsewhere herein, can be used for, but not limited to, modulating Dkk-1 activity, alleviating Dkk-1 mediated inhibition of osteogenesis, treating osteolytic lesions in multiple myeloma and modulating proliferation of a cell. The skilled artisan would understand, based upon the disclosure provided herein, that the present invention encompasses a method of identifying a composition that inhibits Dkk-1 function on the canonical Wnt signaling pathway and/or inhibiting the effects of Dkk-1.

As discussed elsewhere herein, the Dkk-1 antagonist includes, but is not limited to, a peptide corresponding to the LRP-6 binding site of Dkk-1 or an antibody that specifically binds to Dkk-1. Compositions of the present invention having an inhibitory effect on Dkk-1 include but are not limited to, lithium and other GSK3β inhibitor.

As disclosed elsewhere herein, the compositions of the present invention can be used for a variety of purposes including but not limited to modulating Dkk-1 activity, modulating the Wnt signaling pathway, modulating cellular proliferation, treating an osteolytic lesion in multiple myeloma, enhancing osteogenesis, and diagnostic purposes. Preferably, the present invention encompasses any peptide identified by the methods described elsewhere herein, as exemplified by, among others, the peptide sequences of SEQ ID Nos. 11-17. Preferably, the peptide is peptide A (GNDHSTLDGYSRRTTLSSKM; SEQ ID NO:11).

B. Methods Relating to the Use of a Dkk-1 Antagonist for Treating an Osteolytic Lesion in Multiple Myeloma.

The invention further encompasses a method for inhibiting development and growth of an osteolytic lesion in multiple myeloma. The method comprises administering to a patient, an effective amount of a Dkk-1 antagonist. For instance, the Dkk-1 antagonist can be administered to an individual (e.g., a mammal, such as a human) suffering from an osteolytic lesion. The Dkk-1 antagonist of the present invention can also be administered to an individual to prevent an osteolytic lesion. As discussed elsewhere herein, a Dkk-1 antagonist interacts with Dkk-1 and interferes with its function or blocks or neutralizes a relevant activity of Dkk-1. In addition to using a Dkk-1 antagonist as described elsewhere herein, the present invention also encompasses using compositions capable of reversing the effects of Dkk-1 to treat an osteolytic lesion in multiple myeloma in a mammal. Thus, one skilled in the art based upon the present disclosure would appreciate that any composition capable of reversing the effects of Dkk-1 on the Wnt signaling pathway is a candidate for the use in the treatment of an osteolytic lesion. An example of a composition capable of reversing the effects of Dkk-1 on the Wnt signaling pathway is a GSKβ inhibitor. Preferably, the GSKβ inhibitor is lithium.

In sum, the invention includes using a Dkk-1 antagonist including but not limited to a peptide corresponding to the LRP-6 binding site of Dkk-1, an antibody that specifically binds to Dkk-1, and a composition capable of reversing the effects of Dkk-1. One skilled in the art once armed with the present disclosure would recognize that the Dkk-1 antagonist of the present invention as well as lithium and a GSK3β inhibitor can be used among others to enhance osteogenesis, modulate proliferation and differentiation of a cell, and treat an osteolytic lesion in multiple myeloma.

C. Methods of Diagnosis and Assessment of Therapies

The present invention includes methods of diagnosing certain diseases, disorders, or conditions such as, but not limited to, using an anti-Dkk-1 antibody to assess the level of Dkk-1 to detect the presence of an osteolytic lesion in a mammal.

An embodiment of the present invention encompasses a method for detecting the presence or onset of an osteolytic lesion in a mammal comprising the steps of: (a) measuring the amount of Dkk-1 in a sample from said mammal; and (b) comparing the amount determined in step (a) to an amount of Dkk-1 present in a standard sample. An increased level in the amount of Dkk-1 in step (a) when compared with the level of Dkk-1 from step (b) is an indication of osteolytic lesions.

VII. Kits

The invention includes various kits which comprise a compound, such as a Dkk-1 antagonist, and/or compositions of the invention, an applicator, and instructional materials which describe use of the compound to perform the methods of the invention. Although exemplary kits are described below, the contents of other useful kits will be apparent to the skilled artisan in light of the present disclosure. Each of these kits is included within the invention.

In one aspect, the invention includes a kit for antagonizing Dkk-1 activity. Another aspect includes a kit for inhibiting an osteolytic lesion. The kit is used pursuant to the methods disclosed in the invention. Briefly, the kit may be used to administer a Dkk-1 antagonist of the invention and/or compositions of the invention, or a biologically active fragment thereof, to a mammal (e.g., a human) having an osteolytic lesion, or at risk of osteolytic lesion. The kit may also be used to administer a Dkk-1 antagonist of the invention, or a biologically active fragment thereof to enhance osteogenesis.

The kit further comprises an applicator useful for administering the Dkk-1 antagonist and/or compositions of the invention to the mammal. The particular applicator included in the kit will depend on, e.g., the method used to administer the Dkk-1 antagonist, as well as the mammal to which the Dkk-1 antagonist and/or compositions of the invention is to be administered, and such applicators are well-known in the art and may include, among other things, a pipette, a syringe, a dropper, and the like. Moreover, the kit comprises an instructional material for the use of the kit. These instructions simply embody the disclosure provided herein.

The kit includes a pharmaceutically-acceptable carrier. The composition is provided in an appropriate amount as set forth elsewhere herein. Further, the route of administration and the frequency of administration are as previously set forth elsewhere herein.

VIII. Enhanced Growth of Adult Stem Cells

In addition to methods and compositions for regulating the effects of Dkk-1 using Dkk-1 antagonists or compositions capable of reversing the effects of Dkk-1 on the Wnt signaling pathway, the present invention also includes a method of enhancing the proliferative and multipotential capacities of MSCs and defines improved conditions for obtaining standardized preparations of human MSCs. The method comprises isolating MSCs from bone marrow aspirate and plating the MSCs at an initial density of at least about 50 cells/cm².

Considerable variations in results obtained using MSCs for cell and gene therapy led to the development of a standardized protocol for preparing and characterizing MSCs, and it was determined that the initial plating density plays a role in developing standardized protocols. The initial plating density may be from about 50 cells/cm² to about 1000 cells/cm². In another embodiment, the initial plating density may be from about 500 cells/cm² to about 1000 cells/cm². Preferably, the initial plating density may be about 50 cells/cm² to about 200 cells/cm². Preferably, the initial plating density is from about 50 cells/cm² to about 80 cells/cm².

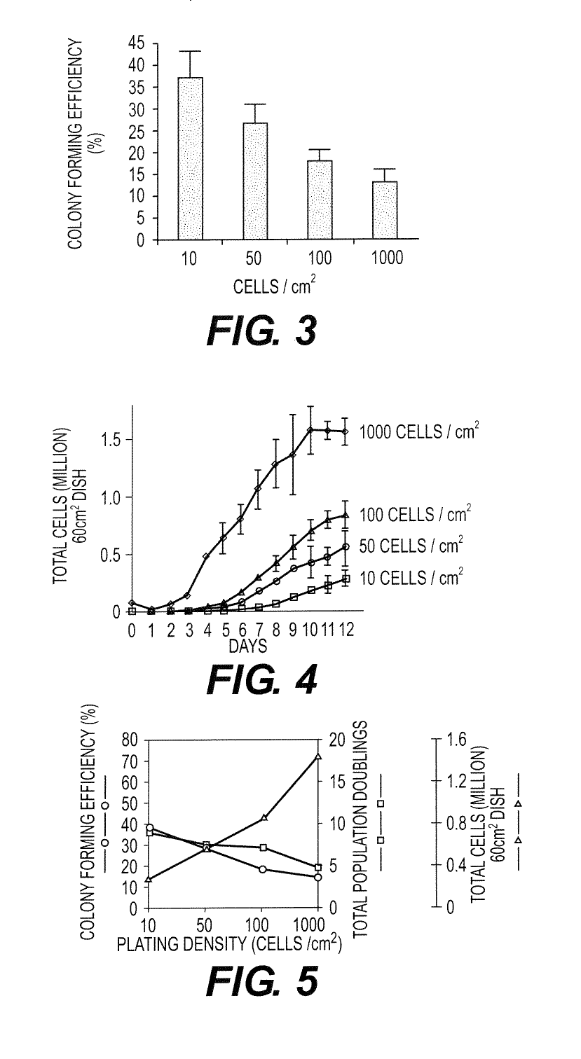

As more fully described below in the Examples, the initial plating density is critical to the production of rapidly expanding and highly multipotential MSCs, and to the colony forming efficiency of the MSCs. Cells plated at a density of at least about 50 cells/cm² expand at a rate of about 200 times over a period of 12 days (FIG. 2), with a maximal doubling rate at 4 days, and have the highest percent colony forming efficiency (FIG. 3).

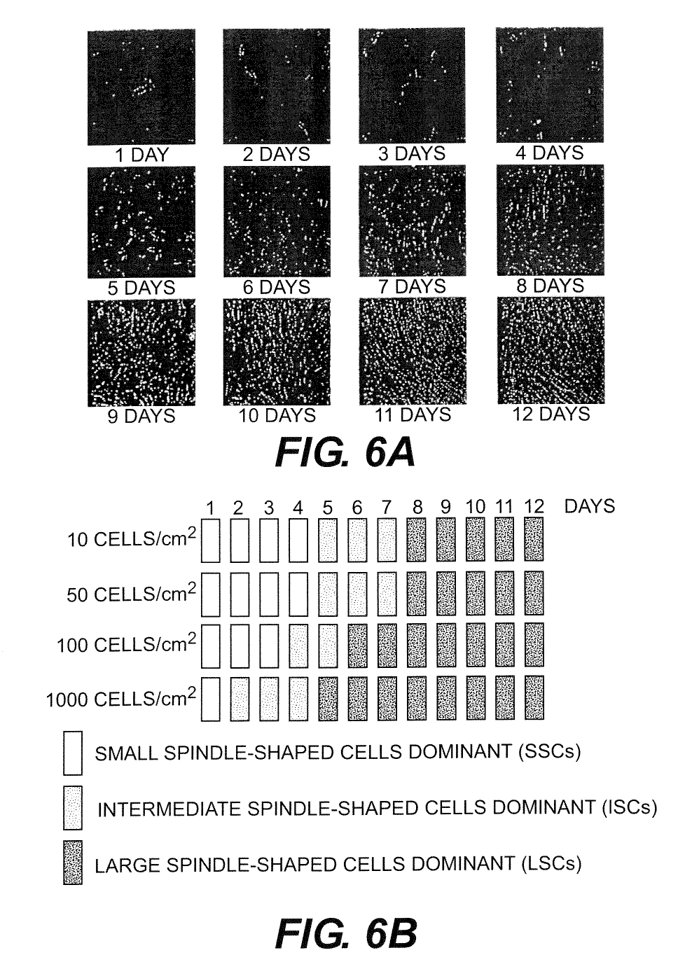

The ability of MSC cultures to generate colonies is closely correlated with their rate of proliferation, their multipotentiality, and their content of rapid, self-renewing cells (RS cells), which are a subpopulation of MSCs having high multipotentiality. RS cells can be further characterized morphologically to small spindle-shaped cells (SSCs), present from Day 1 to 4 in culture, intermediate spindle-shaped cells (ISCs), present from Day 5 to 7 in culture, and large spindle-shaped cells (LSCs), present from Day 8 to 12 (FIG. 6A). Large, flat, mature MSCs (mMSCs) are also present in culture. The present invention demonstrates that cultures having a high percentage of spindle-shaped cells are more highly multipotential than cultures having a high percentage of mMSCs.

As summarized in Table 1, the highest yield of the preparations of MSCs with the highest proportion of SSCs is obtained by plating the cells at 50 cells/cm² and harvesting the cultures after 4 days. The highest yield of the preparations of MSCs with the highest proportion of ISCs is obtained by plating the cells at 1000 cells/cm² and harvesting the cultures after 4 days. However, the fold expansion was significantly less. A more favorable approach is to harvest the cells plated at 50 cells/cm² after 7 days of culture. The fold expansion is much greater than the cells plated at 1000 cells/cm², and the yield is high as well.

When subjected to adipogenic or chondrogenic medium, it was noted that SSCs optimally differentiate into adipocytes, and ISCs optimally differentiate into chondrocytes, indicating that the time differential between maturity in RS cells is directly proportional to the multipotentiality of the cells.

TABLE 1
Optimal conditions to harvest SSCs and ISCs

| | | | Yield per | | | |
|---|---|---|---|---|---|---|
| Initial | Optimal | Fold | 60 cm² | | | |
| Plating | Time to | Expan- | dish | Major | Optimal | |
| Density | Harvest | sion | (×10³ | Cell | differentiation | |
| cells/cm² | (days) | (folds) | cells) | Type | Adipo | Chondro |
| 10 | 4 | 4 | 4 | SSC | + | |
| 10 | 7 | 64 | 38 | ISC | | + |
| 50 | 4 | 5 | 24 | SSC | + | |
| 50 | 7 | 58 | 175 | ISC | | + |
| 100 | 3 | 2 | 12 | SSC | + | |
| 100 | 5 | 13 | 77 | ISC | | + |
| 1000 | 4 | 8 | 480 | ISC | | + |

The present invention also includes a new single-cell colony assay to detect cell differentiation. Briefly, cells are initially plated at from about 50 cells/cm² to about 1000 cells/cm². The cells are sorted with a cell sorter to obtain single cell cultures, and the cells are cultured for 10 to 14 days in complete MSC medium. Colony production is assayed with crystal violet staining. The improved method allows for better reproducibility of the assay by assaying single cells. The cells can then be cultured in a differentiation medium to differentiate into specific cell types.

In addition to assaying the colony forming efficiency of cells, the method can also be used to detect highly clonogenic MSCs (RS cells) in MSC cultures. The method includes analyzing the forward scatter (FS) and side scatter (SS) light pattern of single cells in culture using a closed stream flow cytometer. Use of an open stream flow cytometer did not yield reproducible results in the experiments presented here, but this does not necessarily indicate that an open stream flow cytometer will not work with the present invention. Further testing is necessary to determine the reproducibility of the open stream flow cytometer.

In Example 2 presented herein, the improved assay for detecting clonogenic MSCs is taught. The low FS and SS light assay was used to isolate a sub-fraction of rapidly self-renewing cells (RS cells) that was up to 95% clonogenic and multipotential for differentiation.

The present invention also relates to methods and compositions for enhancing the growth of adult MSCs by enhancing the growth medium. Specifically, the present invention demonstrates that a previously known polypeptide called Dickkopf-1 (Dkk-1) is synthesized and secreted during the most rapid growth in culture of MSCs. Thus, supplementing the growth medium with Dkk-1 leads to extended periods of rapid growth.

MSCs begin to secrete Dkk-1 at the end of the lag phase of growth (about 3 to 5 days from when the cells are first plated in tissue culture) and cease synthesizing and secreting it as the growth of the cells slows down. Dkk-1 is an inhibitor of the Wingless (Wnt) signaling pathway. An increase in Wnt signaling has been shown to increase proliferation of hematopoietic stem cells from bone marrow (Austin, et al., Blood 89:3624-3635 (1999)). The results demonstrated herein indicate that inhibition of the same Wnt pathway increases expansion of MSCs.

MSCs treated with 10 micrograms per milliliter of Dkk-1 antibody produced about 40% less cells than those left untreated, i.e., than those cells which produced and secreted the Dkk-1 protein during the lag phase.

Supplementing MSC growth medium with about 0.01 micrograms per milliliter to about 0.1 micrograms per milliliter of recombinant Dkk-1 produces a larger population of cells in a shorter period of time. In addition, Dkk-1 supplementation allows the MSCs to produce larger colonies. Therefore, adding Dkk-1 to the growth medium when culturing MSCs produces a clinically therapeutic number of cells for administration in gene or cell therapy applications in a much shorter period of time.

It has recently been discovered that certain peptides derived from the Dkk-1 protein serve as specific markers for RS cells in a population of MSCs. These peptides can serve as a purifying mechanism to selectively bind and isolate early progenitor MSCs (RS cells).

Recombinant Dkk-1 peptides can be generated, despite the fact that recombinant Dkk-1 itself is difficult to generate in large quantities because of the high number of cysteine-rich domains that fold improperly. Recombinant Dkk-1 peptides are preferably derived from the domain of Dkk-1 that appears to bind the co-receptor lipoprotein-related receptor protein-6 (LRP-6) of the Wnt signaling pathway. In one embodiment, Dkk-1 peptides are synthesized by substituting serine in place of cysteine in this domain of the Dkk-1 protein. Binding studies between the recombinant peptides and a population of MSCs can then be performed using, for example, a commercially available streptavidin-biotin system in combination with a fluorescent tag in order to identify and isolate RS cells.

In addition, these peptides can also serve as agonists of Dkk-1, thus, being used to increase the rate of proliferation of RS cells, as more fully discussed herein.

Also important in the production MSCs for successful cell and gene therapy applications is the ability to reduce immunogenicity as much as possible. This can be accomplished in part by using autologous MSCs. However, a large number of MSCs is usually required for use of the cells in cell or gene therapy applications, which means that autologous MSCs must be cultured in vitro to obtain an appropriate number of cells. During in vitro culture, the MSCs may internalize the fetal calf serum (FCS) or other animal serum used in the growth media, causing an increase in immunogenicity of the MSCs with respect to the patient from which the original MSCs were obtained.

To solve this problem, the present invention provides a method of removing up to 99.9% internalized animal serum, thereby reducing the immunogenicity of the MSCs and enhancing the success rate for cell and/or gene therapy applications.

The method includes culturing cells with an autologous human serum supplemented with epidermal growth factor (EGF) and basic fibroblast growth factor (bFGF), hereinafter called AHS⁺. In another embodiment, the method includes culturing cells with a heterologous serum. Preferably, the cells are cultured with heterologous serum that is prepared fresh.

Preferably, the EGF is present at a concentration of about 10 nanograms per milliliter and the bFGF is present at a concentration of about 10 nanograms per milliliter. Other concentrations of EGF and bFGF are useful in the present invention, such as from about 0.1 nanogram per milliliter to about 100 nanograms per milliliter. Preferably, the range is from about 1 nanogram per milliliter to about 50 nanograms per milliliter. More preferably, the range is from about 5 nanograms per milliliter to about 20 nanograms per milliliter.

Other growth factors known in the art are also useful in the present invention, such as, for example, platelet-derived growth factor (PEGF).

Also included in the present invention is a novel growth factor medium having autologous serum supplemented with growth factor and Dkk-1 protein. In one embodiment of the invention, the supplemental growth factor is preferably a combination of EGF and bFGF. Preferably, the concentrations of each of EGF and bFGF is about 10 nanograms per milliliter each. Other concentrations of EGF and bFGF are useful in the present invention, such as from about 0.1 nanogram per milliliter to about 100 nanograms per milliliter. Preferably, the range is from about 1 nanogram per milliliter to about 50 nanograms per milliliter. More preferably, the range is from about 5 nanograms per milliliter to about 20 nanograms per milliliter.

In another embodiment of the present invention, the Dkk-1 protein is added to the growth medium at a concentration of 0.01 microgram per milliliter up to about 0.1 microgram per milliliter. Preferably, the Dkk-1 protein is added at a concentration of 0.01 microgram per milliliter.

In a preferable embodiment, autologous marrow stromal cells are initially plated at a density of about 50 cells/cm² and are cultured in a growth medium containing about 0.01 microgram per milliliter Dkk-1 protein and autologous serum supplemented with about 10 nanograms per milliliter each of EGF and bFGF. Culturing the cells in this manner produces the greatest number of multipotential RS cells in the shortest period of time.

The present invention also teaches a method for producing a population of early progenitor MSCs in culture. The method includes depriving a population of MSCs of serum for a period of time, and then recovering the MSCs in medium containing serum. The serum-free medium does not usually contain growth factors or other supplements. The MSCs can be grown in the serum-free medium for about 1 to about 5 weeks, more preferably, from about 2 to about 4 weeks, and more preferably, about 3 weeks. After the serum-free incubation period, the MSCs can be introduced to medium including serum in order to grow and propagate. The MSCs can be cultured in medium containing serum for about 2 to about 7 days in order to induce morphological and/or genotypic changes in the MSCs. Preferably, the MSCs are incubated in serum-containing medium for about 5 days.

In Example 6 and the experiments described therein, MSCs that remained functional after being cultured in serum-free medium displayed remarkable morphological changes when introduced into medium containing serum. After about 5 days of culture with serum, the MSCs changed from large, senescent cells to spindle shaped, characteristic of the early progenitor MSCs. The MSCs had the ability to propagate in medium containing serum through about 13 to about 15 passages. Expression of genes characteristic of early progenitor cells also occurred during the recovery incubation in serum-containing medium. For example, Oct-4, hTERT, and ODC antizyme (see FIG. 35), genes that are typically expressed during the embryonic stage, were all upregulated.

In addition to the expression of early progenitor MSCs, the serum-deprived MSCs had extended telomeres, indicating that the aging process of these MSCs was inhibited.

IX. Modulating Cellular Proliferation

As discussed elsewhere herein, the present invention encompasses methods of enhancing the proliferation and multipotential capacities of MSCs, for example supplementing MSC growth medium with about 0.01 micrograms per milliliter to about 0.1 micrograms per milliliter of recombinant Dkk-1 produces a larger population of cells in a shorter period of time. In addition, Dkk-1 supplementation allows the MSCs to produce larger colonies. Therefore, adding Dkk-1 to the growth medium when culturing MSCs produces a clinically therapeutic number of cells for administration in gene or cell therapy applications in a much shorter period of time.

In addition to enhancing the proliferation of MSCs using compositions and methods described elsewhere herein, one skilled in the art would appreciate based upon the present disclosure that the proliferation of MSCs can also be retarded using Dkk-1 antagonists and/or compositions capable of inhibiting the effects of Dkk-1 on the Wnt signaling pathway. Therefore, the present invention encompasses methods for regulating both the proliferation and differentiation of MSCs. That is, the proliferation of MSCs can be enhanced and retarded using the compositions and methods disclosed elsewhere herein. Preferably, a Dkk-1 agonist can be used to enhance the proliferation of a cell and subsequently the proliferating cell can then be incubated with a Dkk-1 antagonist or a composition capable of reversing the effects of Dkk-1 to retard the proliferation of the cell. Conversely, a proliferating cell can be incubated with a Dkk-1 antagonist or a composition capable of inhibiting the effects of Dkk-1 to retard the proliferation of the cell, and then if desired the cell can be further incubated with a Dkk-1 agonist to enhance the proliferation of the cell.

The following examples are presented to illustrate the present invention. It should be understood that the invention should not to be limited to the specific conditions or details described in these examples. Throughout the specification, any and all references to a publicly available document, including but not limited to a U.S. patent, are specifically incorporated by reference.

### EXAMPLES

### Example 1

### Standardization for Characterizing MSCs

The materials and Methods used in the experiments presented in this Example are now described.

Isolation and Cultures of Human MSCs

To isolate human MSCs, 2 to 10 milliliters of bone marrow aspirates were taken from the iliac crest of normal adult donors after informed consent and under a protocol approved by an Institutional Review Board. Nucleated cells were isolated with a density gradient (Ficoll-Paque, Pharmacia, Piscataway, N.J.) and resuspended in complete culture medium (alpha-MEM, GIBCO BRL; 20% fetal bovine serum, FBS lot-selected for rapid growth of MSCs (Atlanta Biologicals, Norcross, Ga.) 100 units per milliliter penicillin; 100 micrograms per milliliter streptomycin; and 2 millimolar L-glutamine, (GIBCO BRL, Rockville, Md.).

All of the nucleated cells were plated in 20 milliliters of medium in a culture dish and incubated at 37° C. with 5% CO₂. After 24 hours, non-adherent cells were discarded, and adherent cells were thoroughly washed twice with phosphate-buffered saline. The cells were incubated for 4-7 days, harvested with 0.25% trypsin and 1 millimolar EDTA for 5 minutes at 37° C., and replated at 3 cells/cm² in an intercommunicating system of culture flasks (6300 cm² Cell Factory, Nunc, Rochester, N.Y.). After 7 to 12 days, the cells were harvested with trypsin/EDTA, suspended at 1×10⁶ cells per milliliter in 5% DMSO and 30% FBS, and frozen in 1 milliliter aliquots in liquid nitrogen (passage 1). To expand a culture, a frozen vial of MSCs was thawed, plated in a 60 cm² culture dish, and incubated for 4 days (passage 2).

Culture Density and Proliferation

MSCs were cultured at 10 cells/cm², 50 cells/cm², 100 cells/cm², and 1000 cells/cm² in 60 cm² dishes (Corning, Rochester, N.Y.). Cell morphology was then observed and pictures were taken over the next 12 days under light microscopy. Each day, cells from 3 plates from each culture density were harvested, and counted with a hemacytometer. For colony forming assay, 100 cells of MSCs cultured for 12 days were transferred into 60 cm² dishes and cultured for 14 days. Then cell colonies were stained with 0.5% crystal violet in methanol for 5 minutes. The cells were washed twice with distilled water and visible colonies were counted.

Adipogenesis after High Density Plating Assay

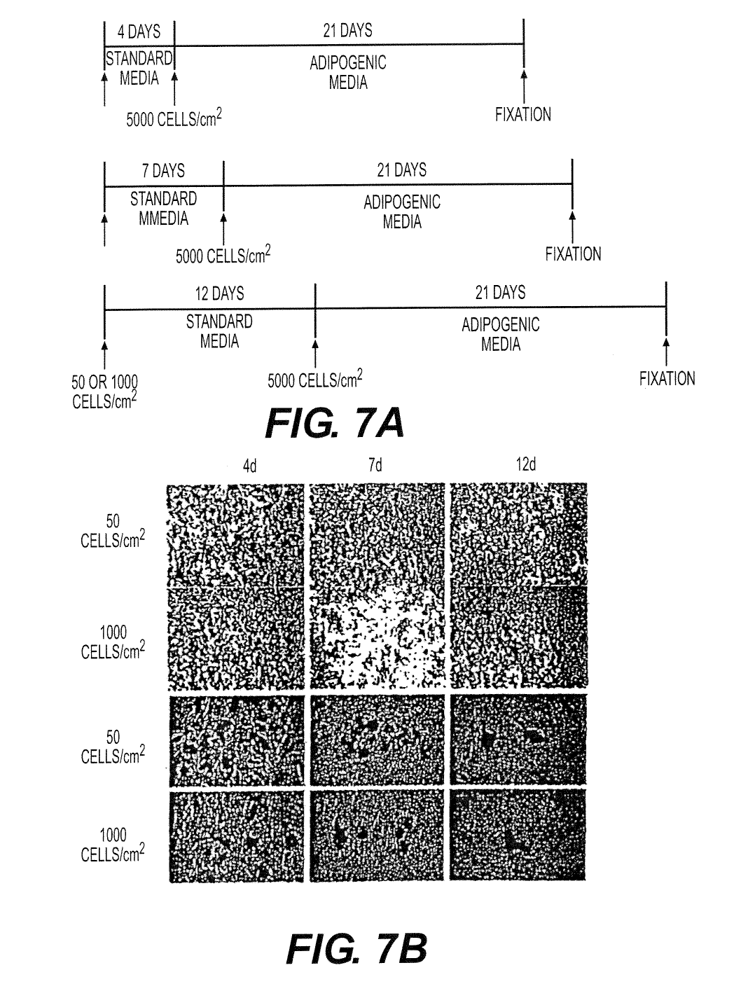

MSCs were plated at 50 cells/cm² or 1000 cells/cm², cultured in complete culture media for 4, 7, and 12 days in 60 cm² dishes, and then replated and cultured in adipogenic media containing complete medium supplemented with 0.5 micromolar dexamethasone (Sigma, St. Louis, Mo.), 0.5 micromolar isobutylmethylxanthine (Sigma, St. Louis, Mo.), and 50 micromolar indomethacin (Sigma, St. Louis, Mo.). After 21 days, the adipogenic cultures were fixed in 10% formalin for over 1 hour and stained with fresh oil-red-o solution for 2 hours (FIG. 7A). The oil red-o solution was prepared by mixing 3 parts stock solution (0.5% in isopropanol; Sigma, St. Louis, Mo.) with 2 parts water and filtering through a 0.2 micron filter. Plates were washed three times with PBS and observed microscopically under low and high magnification.

Adipogenesis in Colony-Forming Assay

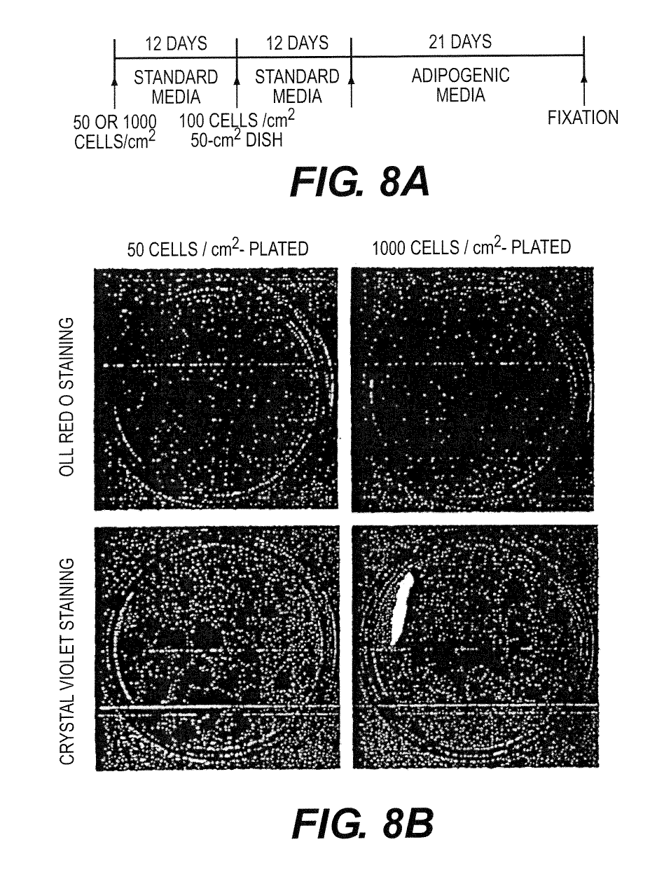

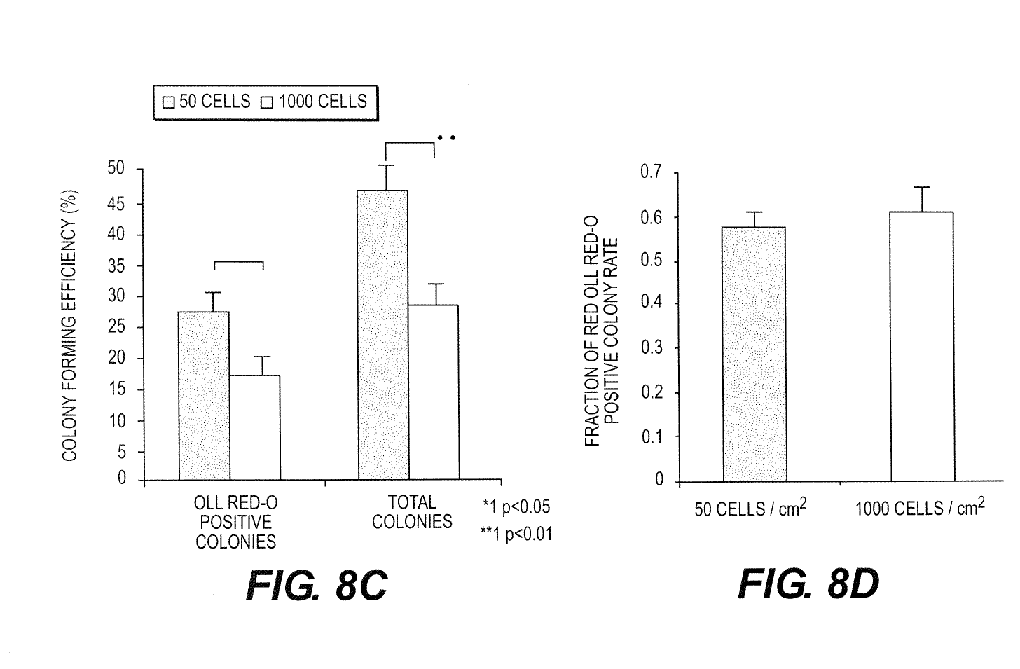

MSCs were plated at 50 cells/cm² or 1000 cells/cm² and cultured in complete media for 12 days. Then 100 cells of MSCs were transferred into 60 cm² dishes and cultured in complete media for 12 days. Then the cells were cultured in adipogenic media for additional 21 days. The adipogenic cultures were fixed in 10% formalin and stained with fresh oil-red-o solution (FIG. 8A) and the number of oil red-o positive colonies was counted. Less than 2 millimeter-diameter or faint colonies were excluded. Then the same adipogenic cultures were stained with crystal violet and the number of total cell colonies was counted.

Chondrogenesis

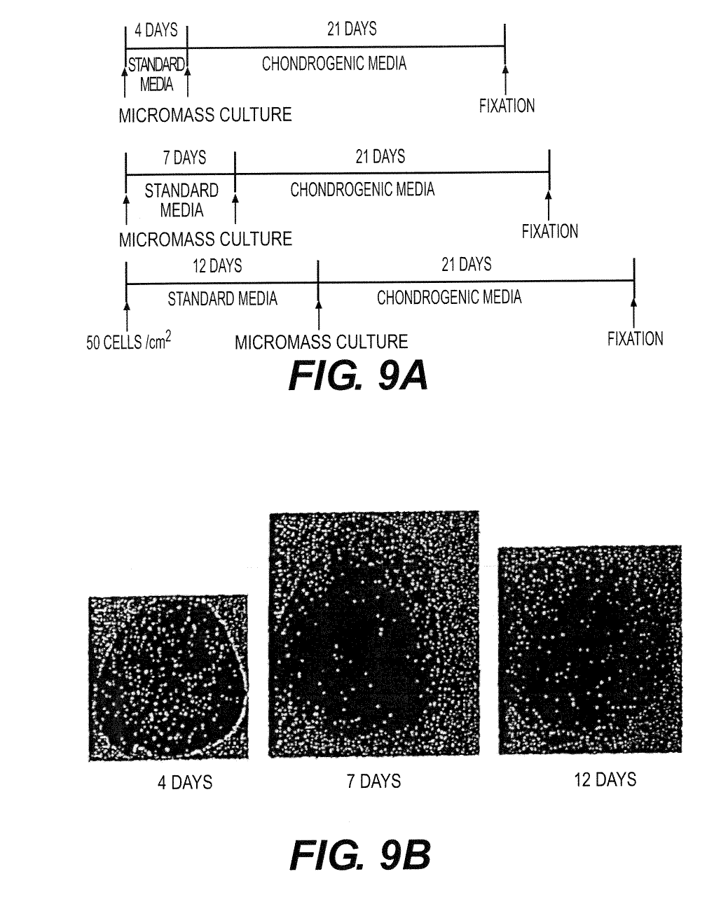

MSCs were plated at 50 cells/cm² and cultured in complete media for 4, 7, or 12 days. For chondrocyte differentiation, a micromass culture system was used. Approximately 200,000 MSCs were placed in a 15 milliliter polypropylene tube (Falcon, Bedford, Mass.), and pelleted into micromasses after centrifugation. The pellet was cultured for 21 days in chondrogenic media that contained 500 micrograms per milliliter BMP-6 (R&D systems, Minneapolis, Minn.) in addition to high-glucose DMEM supplemented with 10 nanograms per milliliter TGF-beta-3, 10⁻⁷ M dexamethasone, 50 micrograms per milliliter ascorbate-2-phosphate, 40 micrograms per milliliter proline, 100 micrograms per milliliter pyruvate, and 50 milligrams per milliliter ITS+™ Premix (Becton Dickinson, Lincoln Park, N.J.) (FIG. 9A). For microscopy, the pellets were embedded in paraffin, cut into 5 micrometer sections and stained with toluidine blue sodium borate.

The Results of the experiments presented in this Example are now described.

Effect of Plating Density on Expansion of MSCs in Culture

To select a preparation of MSCs for further study, bone marrow aspirates were obtained from 5 volunteers, nucleated cells were isolated with a density gradient, and the cells were plated at high density for 4 to 7 days. The adherent cells were removed with EDTA/trypsin, replated at 3 cells/cm² and incubated for 7 to 12 days before being stored frozen in aliquots of about 1 million cells (Passage 1 cells). Frozen vials from each preparation were thawed, replated at high density for 4 days (Passage 2) and then replated at 3 to 50 cells/cm² (Passage 3) for 7 days. Three of the cells expanded slowly but two of the five preparations expanded at rapid rates of over 50-fold in 7 days after plating at 50 cells/cm². One of the rapidly expanding preparations (89 L) was used at Passage 3 cells for all the experiments presented here.

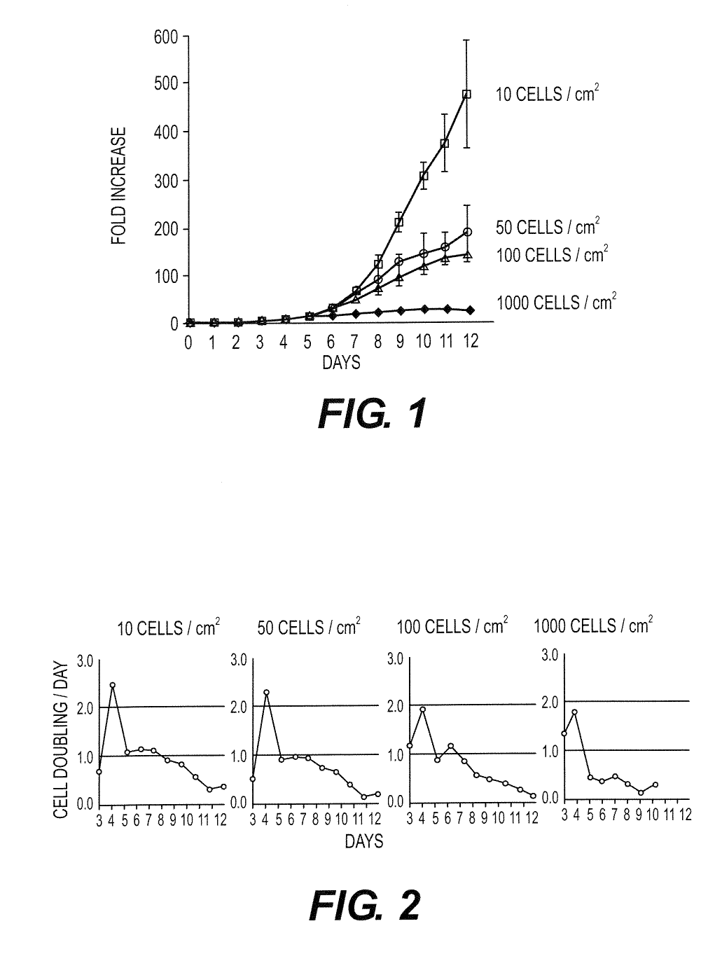

After plating of Passage 3 cells at densities ranging from 10 to 1,000 cells/cm², all the cultures demonstrated a long lag period so that there was little difference in the fold increases of the cells before 7 days (FIG. 1). After 8 days, the expansion was much larger with cultures plated at the lower densities. Cells initially plated at densities of 10 cells/cm² expanded about 500-fold in 12 days whereas cells plated at 1,000 cells/cm² expanded about 30-fold.

The peak doubling rate per day for cells plated at either 10 or 50 cells/cm² was about 2.5, indicating that the average doubling time on Day 4 was about 10 hours (FIGS. 2A-2D). The peak doubling rate per day was less in cells plated at 100 or 1,000 per cm² but the peak rate was still observed on Day 4. The potential of the cells to generate colonies (colony-forming units or CFU) was critically dependent on the initial plating density (FIG. 3). As expected, the yield of cells per culture plate was much larger at the higher initial plating densities (FIG. 4). After 12 days in culture, the total population doublings were 8.9 for cells initially plated at 10 cells/cm², 7.5 for cells plated at 50 cells/cm², 7.1 for cells plated at 100 cells/cm², and 4.6 for cells plated at 1000 cells/cm² (FIG. 5).

Previous observations with early and late passage cultures suggested that the multipotentiality of human MSCs was closely correlated to CFU values of the cultures. Therefore, the data obtained here suggested that it was necessary to make compromise among the three conditions in preparing cultures of MSCs enriched for the earliest progenitors: (a) the yields of cells per plate, (b) the CFU values, and (c) the total population doublings (FIG. 5).

Morphology of MSCs in Low Density Cultures

It was previously confirmed that early passage cultures of MSCs contain at least two morphologically distinct cell types: Small, spindle-shaped cells that are rapidly self-renewing (RS cells) and large, flat cells that appear to be mature MSCs (mMSCs). In the present experiment, early passage MSCs were examined and morphologically distinct sub-types of spindle-shaped cells were identified: (a) Small, spindle-shaped cells (SSCs) seen in very early cultures (see Days 1 to 4 in FIG. 6A); (b) intermediate spindle-shaped cells (ISCs; see Days 5 to 7 in FIG. 6A); and (c) large spindle-shaped cells (LSCs; see Days 8 to 12 in FIG. 6A). Multilayered LSCs were observed after Day 11.

The sub-types of the spindle-shaped cells appeared in the cultures in a defined sequence. The time required for the transition from SSCs to ISCs, and ISCs to LSCs was more rapid with cells initially plated at higher densities (FIG. 6B). As we reported previously, cultures enriched for SSCs had a greater potential than cultures enriched for mMSCs to differentiated into adipocytes and osteoblasts, and cultures enriched for ISCs had a greater potential than cultures enriched for mMSCs to differentiated into chondrocytes. The results suggested therefore that in selecting conditions for expansion of human MSCs in culture, it was also necessary to make a further compromise between yield of cells and recovery of the SSCs and ISCs that are the earliest progenitors by reducing the incubation time depending on the initial plating density.

Adipogenic Potential as Function of Conditions for Expansion of MSCs

To define the adipogenic potential of the expanded MSCs, cells were plated at 50 or 1000 cells/cm² in complete culture medium and expanded for 4, 7 or 12 days before replating at 5,000 cells/cm² in adipogenic medium for 21 days (FIG. 7A). As indicated in FIG. 7B, cells plated at 50 cells/cm² and expanded for 4 days were more adipogenic than cells plated at higher densities. Fewer cells in the cultures became adipocytes if the same cultures were expanded for 7 days or 12 days before transfer to the adipogenic medium. Also, cells initially plated at a density of 1,000 cells/cm² were less adipogenic regardless of how long they were expanded (bottom three panels in FIG. 7B). Therefore, the results suggested that the adipogenic potential of the expanded cells were directly related to their rates of proliferation (FIG. 1), their CFU values (FIG. 3), and the preponderance of SSCs in the cultures (FIG. 6B) at the time the cells are transferred to adipogenic medium.

Correlation between Colonies of Adipocytes and CFUs

Standard assays for adipogenic differentiation of MSCs are complicated by the fact that the cells are replated at near confluency before exposure to adipogenic medium (FIG. 7A).

An assay developed for adipogenesis in single-cell derived colonies of MSCs. MSCs were plated at 50 or 1,000 cells/cm², expanded for 12 days, and then replated at a colony-forming density of 1.7 cells/cm². After incubation for 12 days in standard culture medium so that the cells formed colonies, the cultures were transferred to adipogenic medium for another 21 days (FIG. 8A).

Both the samples initially plated at 50 or 1,000 cells/cm² generated colonies of adipocytes (FIG. 8B, upper two panels). The adipocytic colonies from both samples were of about the same size, but the cells initially plated at 50 cells/cm² generated a larger number of colonies (FIG. 8C). Staining of the same plates with crystal violet indicated, as expected (FIG. 3), that the cells initially plated at 50 cells/cm² had a higher CFU value (FIG. 8B, bottom two panels; FIG. 8C). Of special interest was that the fraction of colonies that became adipocytes was the same with both samples (FIG. 8D). Therefore, the results demonstrated that with both samples, about 60% of the cells that were capable of generating single-cell derived colonies with adipogenic potential.

Correlation Between Conditions for Expansion and Chondrogenic Potential of MSCs

To assay for the chondrogenic potential of the cells, MSCs were plated at 50 cells/cm², expanded for 4, 7, or 12 days, and pelleted into micromasses of about 200,000 cells each before exposure to chondrogenic medium for 21 days (FIG. 9A). The cells that were expanded for 7 days (ISCs) formed larger cartilage pellets than cells expanded for either 4 days (SSC5) or 12 days (LSCs) (FIG. 9B). Also, the cells expanded for 12 days formed larger cartilage pellets than cells expanded for 4 days. Therefore, the results suggested that the cells with the greatest chondrogenic potential were slightly later stage progenitors (ISCs) than the cells with the greatest potential to generate adipocytes (SSCs) (compare FIG. 9B with FIG. 7B).

### Example 2

### Enhanced Method for Characterizing RS Cells

The Materials and Methods used in the experiments presented in this Example are now described.

Human MSCs were prepared as described above.

All the nucleated cells (30 to 70 million) were plated in a 145 cm² dish in 20 milliliters complete medium: alpha-MEM (GIBCO BRL, Rockville, Md.); 20% fetal bovine serum, FBS lot-selected for rapid growth of MSCs (Atlanta Biologicals, Norcross, Ga.); 100 units per milliliter penicillin; 100 micrograms per milliliter streptomycin; and 2 millimolar L-glutamine (GIBCO BRL, Rockville, Md.). After 24 hours at 37° C. in 5% CO₂, adherent cells were discarded and incubation in fresh medium was continued for 4 days. The cells were removed with 0.25% trypsin and 1 millimolar EDTA for 5 minutes at 37° C. and replated at 50 cells/cm² in an interconnecting system of culture flasks (6320 cm² Cell Factory, Nunc, Rochester, N.Y.). After 7 to 9 days, the cells were removed with trypsin/EDTA and in frozen at 10⁶ cells per milliliter liquid nitrogen as Passage 1 cells (P1). For the experiments here, a frozen vial of 10⁶ cells was thawed, plated in 20 milliliters of medium a 145 cm² dish, and incubated for 2 days. The cells (P2) were harvested and then incubated in medium as indicated. The medium was replaced every 3 to 5 days.

For the standardized assay of forward scatter (FS) and side scatter (SS), a closed stream flow cytometer (Epics XL 8C; Beckman-Coulter, Fullerton, Calif.) was standardized with microbeads (7 to 20 micrometers; Dynosphere Uniform Microspheres; Bangs Laboratories Inc., Fisher, Ind.). The pattern of FS/SS was then used to define sub-fractions of cells for sorting with an open stream instrument (FACSVantage SE with Clonesort accessory; Becton-Dickinson, Lincoln Park, N.J.). Staining for senescence-associated beta-galactosidase was carried out with one commercial kit (ImaGene Green TM C 12FDG lacZ Gene Expression Kit; (Molecular Probes, Eugene, Oreg.) and staining for Annexin V with a second commercial kit (Sigma, St. Louis, Mo.). Cell cycle analysis was performed (CycleTEST PLUS DNA Reagent Kit; BD-Biosciences, San Diego, Calif.) with 5×10⁵ trypsinized cells.

To develop an improved assay for CFUs, a fluorescent flow cytometer with an automated cell sorter (FACSVantage SE with Clonesort accessory; Becton-Dickinson, Lincoln Park, N.J.) was used to plate single cells into individual wells of a 96-well microtiter plate. The samples were incubated in complete medium for 10 to 14 days and assayed visible colonies by staining the plates with Crystal Violet.

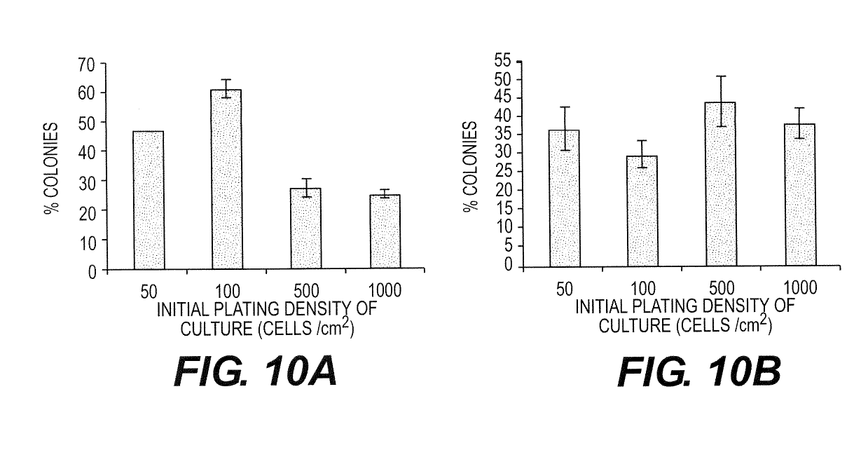

As indicated in FIG. 10A, the single-coil CFU assay (sc-CFU) had a smaller variation than the standard CFU assay. The average coefficient of variation was 4.52 for the sc-CFU and 14.6 for the standard CFU assay. Therefore, the sc-CFU assay was about three times more reproducible. Also, the sc-CFU assay detected important differences not detected by the standard assay (FIG. 10B) between cultures initially plated at 50 or 100 cells/cm² and cultures plated 500 or 1,000 cells/cm². The lower values obtained with the sc-CFU assay for cultures plated at the higher density are consistent with previous observations that cultures plated at higher density show a rapid decrease in the number of multipotential and rapidly self-renewing cells (RS cells).

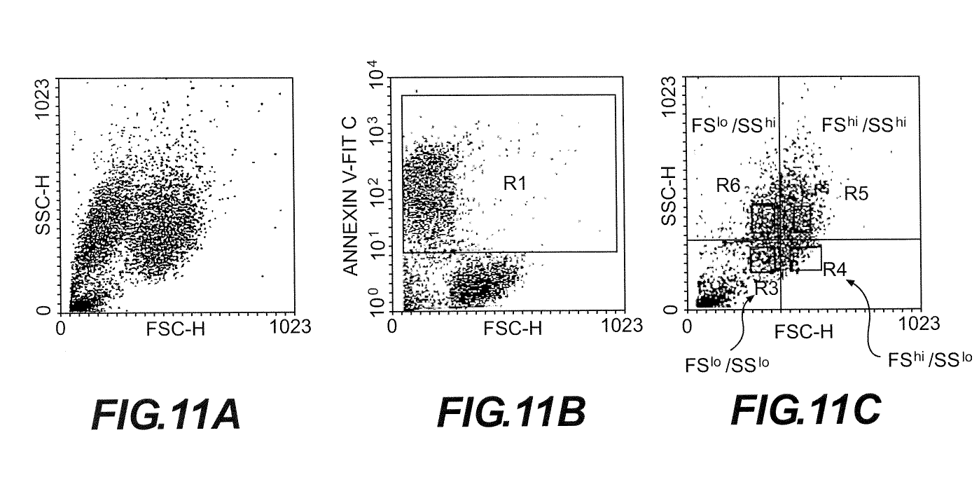

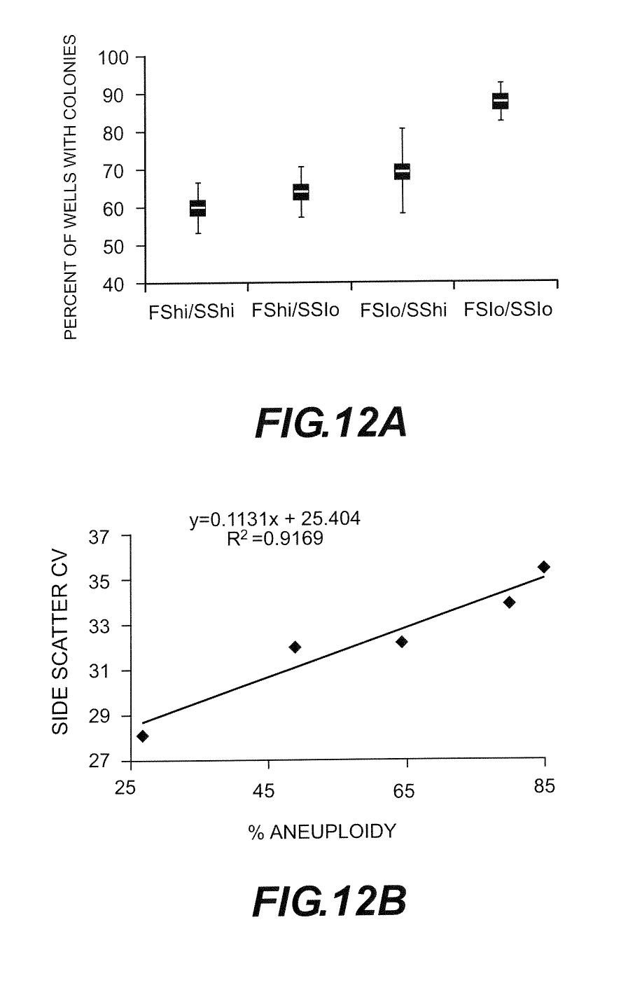

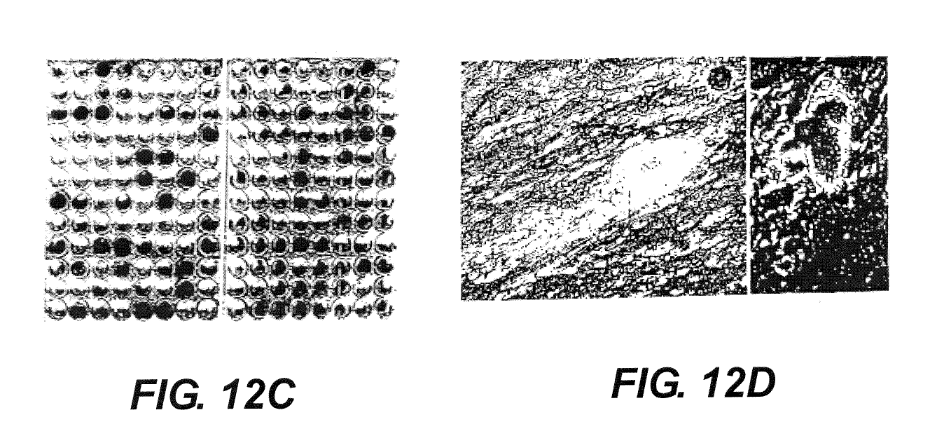

The sc-CFU assay was then used to identify RS cells in cultures of MSCs by FS and SS of light (FIG. 11A). To eliminate cell fragments and apoptotic cells, the cells were stained for Annexin V (FIG. 11B). The remaining Annexin V events were then used to define four sub-fractions of the cells based on FS and SS (FIG. 11C). The exclusion of Annexin V⁺ events proved useful for late passage cultures containing large proportions of large and mature cells with which the Annexin V+events accounted for up to 40% of the total events. It was not essential for early passage; low density cultures under optimal conditions with which the Annexin V+events were less than 1% of the total. Cells gated on the basis of FS^{lo}, SS^{lo}, additional peak adjacent to the 2n peak, suggesting aneuploidy. As indicated in FIG. 12B, there was direct correlation between SS and aneuploidy (Pearson r²=0.92; p=0.0104).

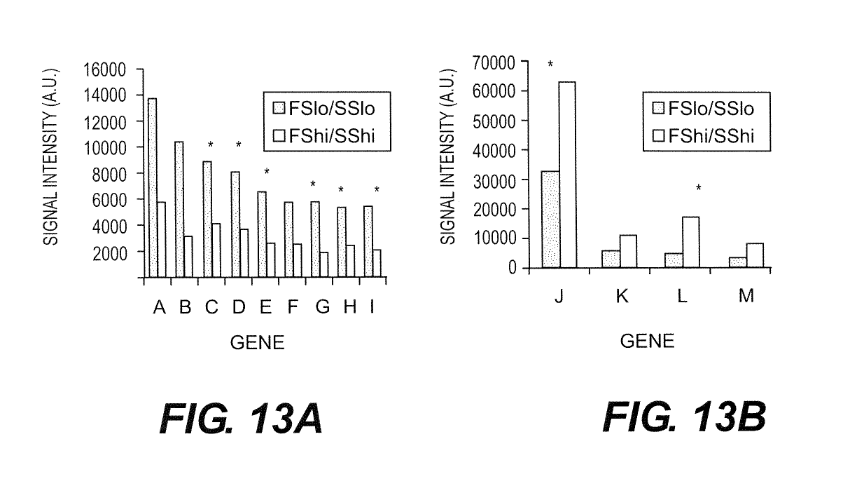

Microarray assays for mRNAs were carried out to compare the FS^{lo}, SS^{lo} cells with the FS^{hi}, SS^{hi} cells (FIG. 12C). The data were first analyzed to select the genes whose signal intensities showed the greatest difference between the two populations. Thirty-four genes differed by an absolute signal log ratio (base 2) of greater than 1, i.e., a greater than 2-fold difference. Of the 13 that showed the greatest differences, 8 were cell cycle related (Table 2). As indicated in FIG. 13, 6 genes that are expressed in cycling cells were expressed at higher levels in FS^{lo}, SS^{lo} cells. In contrast, 2 genes that are expressed in non-cycling cells were expressed at lower levels in the FS^{lo}, SS^{lo} cells.

TABLE 2
Identities of genes shown in FIG. 13.

| Letter | Descriptions |
|---|---|
| A | Cluster Incl. D14657: mRNA for KIAA0101 gene |
| B | Cluster Incl. A.A203476: zx55e01.rl Homo sapiens cDNA |
| C | Cluster Incl. U05340: p55CDC mRNA |
| D | M25753cyclinB |
| E | Cluster Incl. M25753: cyclin B |
| F | Cluster Incl. U10550: Gem GTPase (gem) |
| G | L25876 protein tyrosine phosphatase (CIP2) |
| H | Cluster Incl. U74612: hepatocyte nuclear factor-3/ |
| | forkhead homolog 11A |
| I | L16991 thymidylate kinase (CDC8) |
| J | U03106 wild-type p53 activated fragment-1 (WAF1) |
| K | S37730 insulin-like growth factor binding protein-2 |
| L | AB000584 TGF-betas superfamily protein |
| M | M98539 prostaglandin D2 synthase gene |

As a final step, a rapid and reproducible assay for RS cells in MSC cultures by measuring the light scattering properties of the cells against a standard curve prepared with microbeads of a defined size was developed.

Preliminary experiments demonstrated that the assay was not reproducible if performed in a flow cytometer with an open stream (FACSVantage SE; Becton-Dickinson, Lincoln Park, N.J.); occasionally the values obtained with the microbead standards were the inverse of the known size of the beads. Therefore the assay was standardized in a flow cytometer with a closed stream (Epics XL SC; Beckman-Coulter, San Diego, Calif.).

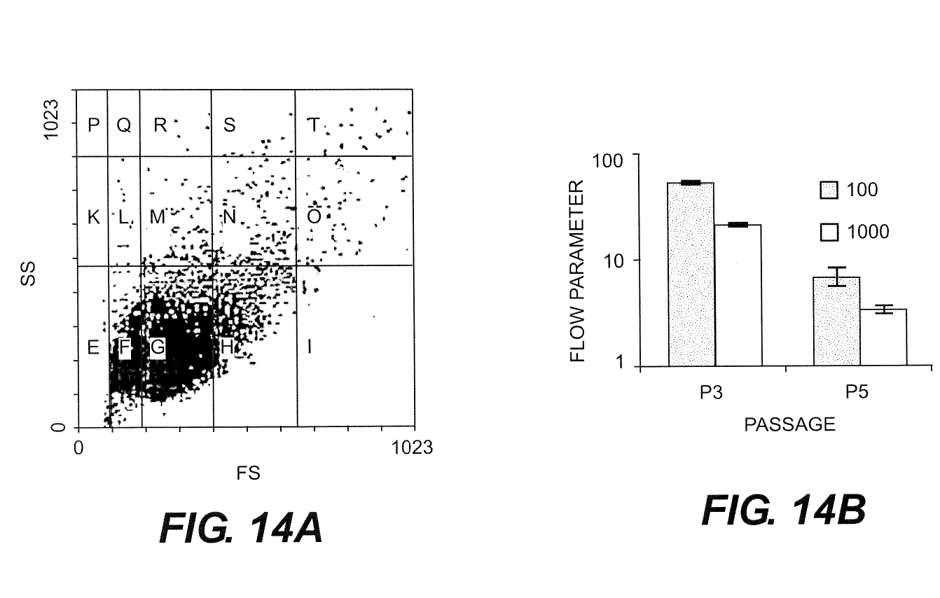

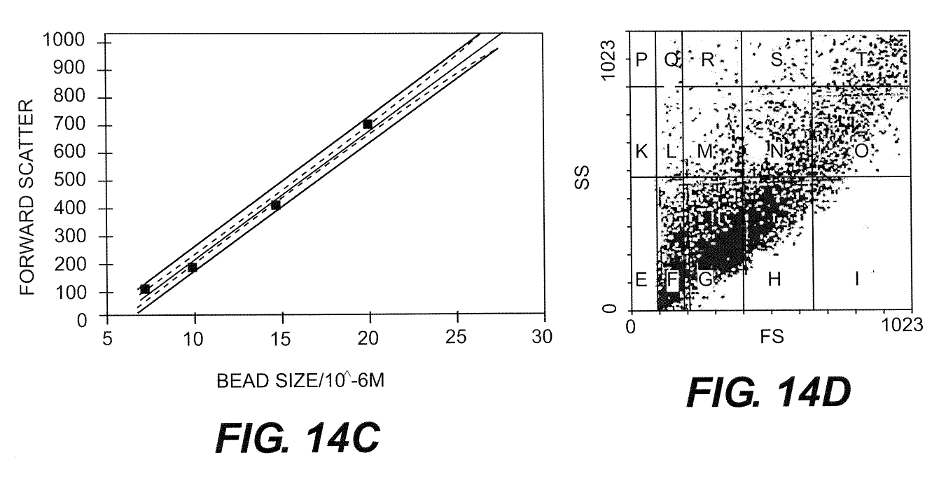

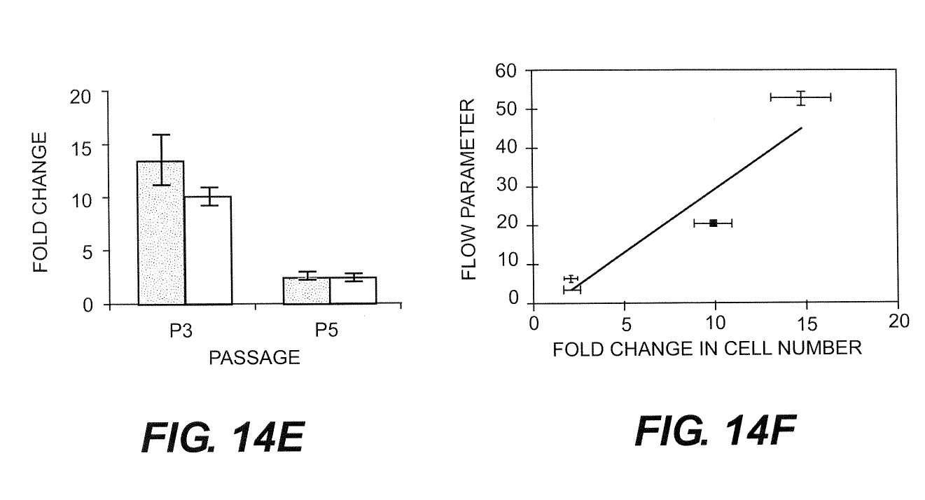

Calibration for FS gave reproducible and linear responses with microbeads ranging in size from 7 to 20 micrometers. The calibration of 55 was standardized with two peaks that were produced by the 55 properties of all the beads in the mixture. The standardized assay was reproducible and readily distinguished early passage cultures enriched for early progenitors and late passage cultures depleted of early progenitors (FIGS. 14A-14D). In addition, the subsequent rate of expansion of a given preparation of MSCs could be predicted on the basis of a flow parameter defined as percent of total Annexin V⁻ cells in region G divided by percent of cells in region T.

Experiments with MSCs are limited by the heterogeneity that is present within single preparations and among different preparations of the cells. Several groups of investigators attempted to characterize human MSCs with antibodies to distinguishing surface epitopes, but it has been difficult to establish that any of the antibodies selectively identifies the earliest progenitors in standard cultures of MSCs.

In the microarray assays carried out here, mRNAs for epitopes for three promising antibodies (SH-2, SH-3 and SH-4) were expressed at about the same levels in FS^{lo}/SS^{lo} cells as in FS^{hi}/SS^{hi} cells. Therefore, the three antibodies are unlikely to distinguish the two populations.

The protocols developed here provide a reproducible assay for the clonogenicity of MSCs, a characteristic that distinguishes early progenitors from more mature progeny in the same cultures and that is closely correlated with their multipotentiality for differentiation. In addition, the standardized assay for FS and SS provides a rapid measure of the fraction of early progenitors in the cultures. A similar protocol to use light scattering properties made it possible to identify early progenitors in cultures of periosteal cells from fetal rat and may be generally useful to assay for the small stem-like cells in a number of adult tissues.

### Example 3

### Dkk-1 Enhances Proliferation of MSCs

Bone Marrow Tissue Culture

Bone marrow aspirates of about 2 milliliters were drawn from healthy donors ranging in age from 19 to 49 years under an Institutional Review Board approved protocol. Plastic adherent nucleated cells were separated from the aspirate and cultured as previously described in DiGirolamo et al., Br. J. Haematol. 107:275-281. After 14 days in culture, adherent cells were recovered from the monolayer by incubation with 0.25% (w/v) trypsin and 1 millimolar EDTA (Fisher Lifesciences; Pittsburgh, Pa.) for 5 to 7 minutes at 37° C. (Fisher Lifesciences; Pittsburgh, Pa.) and re-plated at a density of 100 cells per cm².

The cells were then cultured for various times with a change of media every 2 to 3 days. Cells were radiolabeled at indicated intervals by addition of new media containing 5 microcuries per milliliter [³⁵S]-labeled methionine (Amersham Pharmacia Biotech; Piscataway N.J.). The cultures were allowed to incorporate the label for 48 hours followed by recovery of the cells and media. Other cell lines were acquired from the American Type Culture Collection and handled according to the instructions provided.

Preparation of Labeled Media and Cell Extracts

To remove unwanted cells and debris, the media was filtered through a 0.22 micron pore size membrane (Millipore Corporation; Bedford, Mass.). To remove unincorporated [³⁵S]-methionine the media was diafiltered against 10 volumes PBS (Sigma Aldrich Incorporated; St. Louis, Mo.) using a tangential flow filtration system fitted with 150 cm² PVDF 5 kDa filters (Millipore, Bedford, Mass.). Cells were counted in a hemacytometer followed by lysis in PBS containing 0.01% (w/v) SDS (Sigma Aldrich). The cell lysates were dialyzed against 1000 volumes of 1×PBS for 24 hours using 3500 dalton limiting dialysis cassettes (Pierce Chemical; Rockford, Ill.). Radioactivity was assayed by liquid scintillation counting using 30% scintillant (Scintisafe, Fisher Lifesciences, Pittsburgh, Pa.).

Electrophoretic Analysis and Immunoblotting

Unless otherwise stated, electrophoresis was carried out using commercial reagents and systems (Novex; Invitrogen Corporation; Carlsbad, Calif.). Two microliters of medium were added to 5 microliters of SDS-PAGE sample buffer and 1 microliter of 2-mercaptoethanol (Sigma Aldrich, St. Louis, Mo.). The samples were heated at 100° C. for 2 minutes and electrophoresed on a 4% to 12% NuPage bis-Tris gel using the MES buffering system.

In some experiments, samples were loaded in triplicate and at different dilutions to assess aberrant migration due to the presence of excessive serum albumin. Gels were either silver stained (Silver Quest Staining Kit; Invitrogen, Carlsbad, Calif.) or blotted onto PVDF filters for autoradiography and immunoblofting. For autoradiographic analysis, filters were air dried and exposed to autoradiography film (Kodak Biomax MR; Sigma Aldrich, St. Louis, Mo.). After 2 days exposure, the film was automatically developed using a commercial instrument and reagents (AGFA Corporation, Ridgefield Park, N.J.).

For immunoblotting, filters were blocked in PBS containing 0.1% (v/v) Tween 20 (Sigma, St. Louis, Mo.) for 1 hour. For detection of beta-catenin, blots were probed with an anti-beta-catenin monoclonal antibody at a dilution of 1 to 1000 (clone 5H10Chemicon International; Temecula, Calif.) followed by an anti-mouse peroxidase-conjugated rabbit serum (Sigma Aldrich, St. Louis, Mo.). For detection of Dkk-1, blots were probed in 1 microgram per milliliter of anti Dkk-1 polyclonal antibody (see below) followed by an anti-rabbit peroxidase-conjugated monoclonal antibody (clone RG 96, Sigma Aldrich, St. Louis, Mo.). Positive bands were detected by chemiluminescence in accordance with a previously described procedure (Spees et al. Cell Stress Chaperones, 7:97-106 (2002)).

Electroelution and Tryptic Fingerprinting of Bands

Two hundred microliters of 5-fold concentrated radiolabeled medium were separated by electrophoresis on a 4% to 20% polyacrylamide Tris-glycine preparative gel (Invitrogen, Carlsbad, Calif.). Fifteen fractions were laterally electroeluted into 1 milliliter of 100 millimolar ammonium bicarbonate (pH 8.0) using a whole gel eluter system (BioRad Laboratories; Hercules, Calif.). The fractions were analyzed by SDS-PAGE followed by 10-fold concentration by rotary evaporation (Savant AES 2010 Rotary Evaporation System; Savant Inc., Holbrook, N.Y.).

Samples were proteolytically digested in 50 microliters reactions containing 100 millimolar ammonium bicarbonate (pH 8.0) in the presence of 5 nanograms of agarose-coupled trypsin (Sigma Aldrich, St. Louis, Mo.). The reaction was incubated at 37° C. for 16 hours followed by removal of the trypsin by centrifugation.

Analysis by mass spectrometry was carried out using commercial instruments and reagents (Ciphergen Biosystems Incorporated; Freemont, Calif.). Aliquots (2 microliters each) of digested samples were mixed with 2 microliters of a saturated solution of alpha-cyano-4-hydroxy cinnamic acid in acetonitrile. The mixture was air dried onto silica-coated aluminum mass spectrometry chips and analyzed using a PBS II surface enhanced laser desorbtion ionization (SELDI) time of flight (TOF) chip reader. The program Peptldent (Wilkins & Williams, J. Theor. Biol. 186:7-15 (1997)) was used to analyze triplicate data sets and appropriate controls with settings for the detection of acryl-cisteinyl groups and oxidized methionine residues. Both the Swiss Prot and TREMBL databases were searched for the resulting peptides.

Antibody Production and Purification

A peptide corresponding to a sequence in the 15 residue long sequence in the second cysteine rich domain of Dkk-1, ARHFWSKICKPVLKE (SEQ ID NO:1), was synthesized and conjugated to keyhole limpet hemocyanin (Sigma Genosys; The Woodlands, Tex.). The conjugated peptide was used to immunize two New Zealand white rabbits. Antibodies were purified from 20 milliliter aliquots of post-immune serum by affinity chromatography against the immunizing peptide.

Briefly, 5 milligrams of peptide at a concentration of 1 milligram per milliliter in 100 millimolar sodium bicarbonate (pH 8.2) was cycled through a 1 milliliter NHS-activated Sepharose column (Amersham Pharmacia Biotech, Piscataway, N.J.) for 16 hours at a flow rate of 1 milliliter per minute. The column was then blocked with 500 millimolar Tris HCl (pH 8.0) arid washed with PBS.

For antibody purification, 50 milliliters of a 5 milligram per milliliter solution of post-immune rabbit serum was cycled through the peptide-coupled column for 5 hours. The column was then washed with 50 milliters of PBS following elution of the polyclonal antibodies in 0.5 milliliter fractions with 100 millimolar glycine pH 2.0. The fractions were adjusted to pH 7.4 with 100 millimolar Tris HCl and then visualized by SDS-PAGE prior to use. Using a protocol, Dkk-1 was immunoaffinity purified from 50 milliliters of conditioned medium by affinity chromatography using antibody-coupled NHS-activated Sepharose.

Production of Recombinant Dkk-1

The cDNA encoding human Dkk-1 was prepared by RT-PCR using mRNA from hMSCs. The cDNA was cloned into the prokaryotic expression vector, pET 16b using standard protocols and reagents (New England Biolabs; Beverly, Mass.). The construct was transformed into BL21 (gamma-DE3) E. coli. Unless otherwise stated, all biochemical reagents for the production of recombinant Dkk-1 were acquired from Fisher Scientific (Pittsburgh, Pa.).

A saturated culture of the transformed bacteria were prepared in 50 milliliters of Lauria Bertani (LB) broth containing 100 micrograms per milliliter ampicillin. The overnight culture was added to 1 liter of fresh LB media with ampicillin and allowed to grow to an optical density of 0.6 at 600 nanometers. Isopropyl-beta-thiogalactopyranoside was added to a final concentration of 0.4 millimolar to induce expression of Dkk-1. After 4 hours, the cells were harvested, resuspended in wash buffer (100 millimolar Tris, pH 8.0, 100 millimolar KCl, 1 millimolar EDTA, 0.2% (w/v) deoxycholic acid), and then lysed by sonication.

Inclusion bodies were washed three times by centrifugation in wash buffer and sonicated into 50 milliliters of 100 millimolar Tris pH 8.0 containing 6 molar urea and 0.1 millimolar DTT. The inclusion body solution was added to 4 liters refolding solution (100 millimolar Tris pH 8.0, 100 millimolar KCl, 2% (w/v) N-lauryl sarcosine, 8% (v/v) glycerol, 100 micromolar NiCl₂, 0.01% (v/v) H₂O₂) and incubated for 48 hours at 4° C. with vigorous stirring.

The sample was filtered through a 0.22 square micron membrane and concentrated to 200 milliliters by diafiltration using a tangential flow filtration system fitted with 150 cm² PVDF 5 kDa filters (Millipore, Bedford, Mass.). The sample was then was diafiltered against 40 volumes of 100 millimolar L-arginine HCl (pH 8.7). Histidine-tagged recombinant Dkk-1 was purified by metal ion affinity chromatography as described in Gregory (Structural and functional studies on recombinant human non-collagenous carboxyl terminal (NC1) domain of human type X collagen. Ph. D. Thesis. University of Manchester, UK (1999)), and then dialyzed into 20 millimolar ammonium carbonate at pH 8.7. The pure, dialyzed protein was dried by rotary evaporation (Savant AES 2010 Rotary Evaporation System) in 10 microgram aliquots and stored at −80° C. For tissue culture studies, each aliquot was resuspended in 1 milliliter of alpha-MEM containing 10% (v/v) fetal calf serum (FCS).

Analysis of Colony Size and Proliferation

MSCs were plated at about 0.6 cells per cm² and incubated in complete medium for 17 days. For direct visualization of colonies, a 5% (w/v) solution of crystal violet in methanol (Sigma Aldrich, St. Louis, Mo.) was added to tissue culture dishes previously washed twice with PBS. After 20 minutes, the plates were washed with distilled water and air-dried. Stained colonies with diameters 2 millimeters or greater were counted.

For assay of proliferation, cells were also quantified by fluorescent labeling of nucleic acids (CyQuant dye; Molecular Probes Incorporated; Eugene, Oreg.). hMSCs were plated at 100 cells per cm² into 10 cm² wells and allowed to grow for 4 days. The cells were washed with PBS and medium was added containing the appropriate concentration of Dkk-1 and FCS. The cells were recovered by trypsinization as described above. Fluorescence analysis was carried out using a microplate fluorescence reader (FLₓ800; Bio-Tek Instruments Incorporated; Winooski, Vt.) set to 480 nanometers excitation and 520 nanometers emission.

Quantitative RT-PCR Analysis

Extraction of total mRNA was carried out from 1 million cells (High Pure; Roche Diagnostics; Indianapolis, Ind.). A one tube RT PCR (Titan; Roche Diagnostics) was employed for the synthesis of cDNA and PCR amplification. The following primers were designed for amplification of Dkk-1:

Dkk-1:
ccttctcatatgatggctctgggcgcagcggga
(sense; SEQ ID NO:2)
cctggaggtttagtgtctctgacaagtgtggaa
(antisense; SEQ ID NO:3)
and
GAPDH:
ccccttcattgacctcaact
(sense; SEQ ID NO:4)
cgaccgtaacgggagttgct
(antisense; SEQ ID NO:5).

Reactions were carried out on a thermal cycler (Applied Biosystems 9700; PE Applied Biosystems; Foster City, Calif.) to the following parameters: initial cDNA synthesis, 50° C. for 45 minutes, denature 95° C. for 1 minute, anneal 52° C. for 1 minute and extend 72° C. for 1 minute, for 28 cycles.

Amplification of LRP-6 was achieved using the following primers:

ccacaggccaccaatacagtt
(sense; SEQ ID NO:6)
tccggaggagtctgtacagggaga
(antisense; SEQ ID NO:7)

Reactions were carried out to the following parameters on a thermal cycler (Applied Biosystems 9700): initial cDNA synthesis, 57° C. for 55 minutes, denature 95° C. for 2 minutes, anneal 55° C. for 1 minute and extend 72° C. for 1 minute for 30 cycles. Samples were analyzed by Tris borate EDTA PAGE using commercial systems and reagents (Novex; Invitrogen, Carlsbad, Calif.) followed by ethidium bromide staining (Sigma Aldrich, St. Louis, Mo.). A previously described hybridization ELISA assay (Gregory et al. Anal. Biochem. 296, 114-121 (2001)) was employed to compare the expression of Dkk-1 over time in culture. The following biotinylated oligonucleotides were designed for the ELISA:

Dkk-1:

(SEQ ID NO:8)

biotin-atagcaccttggatgggtatt
GAPDH:

(SEQ ID NO:9)

biotin-catgccatcactgccacccag

Extraction of Cytoskeletal Fractions

Triton-insoluble fractions were prepared in accordance with Ko et al., Am. J. Physiol. Cell Physiol. 279:C147-C157 (2000). Bridily, one half million cells were suspended in 1 milliliters of ice-cold PBS containing a cocktail of protease inhibitors (Roche Diagnostics, Switzerland) with 1% (v/v) Triton X-100 (Sigma Aldrich, St. Louis, Mo.). Lysis was allowed to proceed for 10 minutes on ice followed by a 60-second centrifugation at 800 g to remove particulate bodies. The cytoskeletal pellet was separated from the cytoplasmic fraction by centrifugation at 14,000 g for 15 minutes and resuspended in 1 milliliter 1×SDS-PAGE loading buffer.

Immunocytochemistry

hMSCs in tissue culture dishes were fixed with 4% (v/v) Paraformaldehyde (USB Corporation, Cleveland, Ohio) for 10 minutes at 4° C. and washed with PBS (Fisher Lifesciences, Pittsburgh, Pa.). Sections (30 millimeter×60 millimeter) of the dishes containing the adherent cells were excised using a hot scalpel under constant hydration with PBS. The samples were blocked in PBS containing 0.4% (v/v) Triton X-100 (Sigma Aldrich, St. Louis, Mo.) and 5% (v/v) goat serum (Sigma Aldrich). Anti-beta-catenin (described above) was added in a 1:400 dilution to the slides in block solution. An appropriate concentration of mouse IgG₁ (Cymbus Biotechnology Chandlers Ford, Hants, UK) was used as an isotype control. The samples were incubated for 16 hours at 4° C. followed by washing in PBS. The samples were then incubated for 1 hour in a 1:800 dilution of Alexa-Fluor 594-congugated secondary antibody (Molecular Probes, Eugene, Oreg.). Isotype controls were acquired from Chemicon and Becton Dickinson Slides were washed and mounted with medium containing DAPI (Vector Laboratories Incorporated; Burlingame, Calif.). Immunofluorescence microscopy and digital imaging was carried out using an upright fluorescent microscope (Eclipse 800, Nikon, Japan).

Cell Cycle Analysis

Cells were seeded into 146 cm² tissue culture plates at an initial seeding density of 100 per cm². After four days, the medium was replaced with fresh medium with or without FCS, and the cultures incubated for a further 24 hours. Cells were harvested by trypsinization, washed once with PBS and then cell pellets were frozen at −80° C. For analysis, approximately 500,000 cells were incubated for 30 minutes on ice in a preparatory labeling reagent containing propidium iodide, detergent and RNAase (New Concept Scientific; Niagara Falls, N.Y.). Fluorescent activated cell sorting was carried out using an automated instrument (Epics XL; Beckman Coulter, San Diego, Calif.) and data analyzed using ModFit LT 3.0 software (Verity Software House; Topsham, Me.).

The Results of the experiments presented in this Example are now described.

Conditioned Medium Increases Proliferation of hMSCs

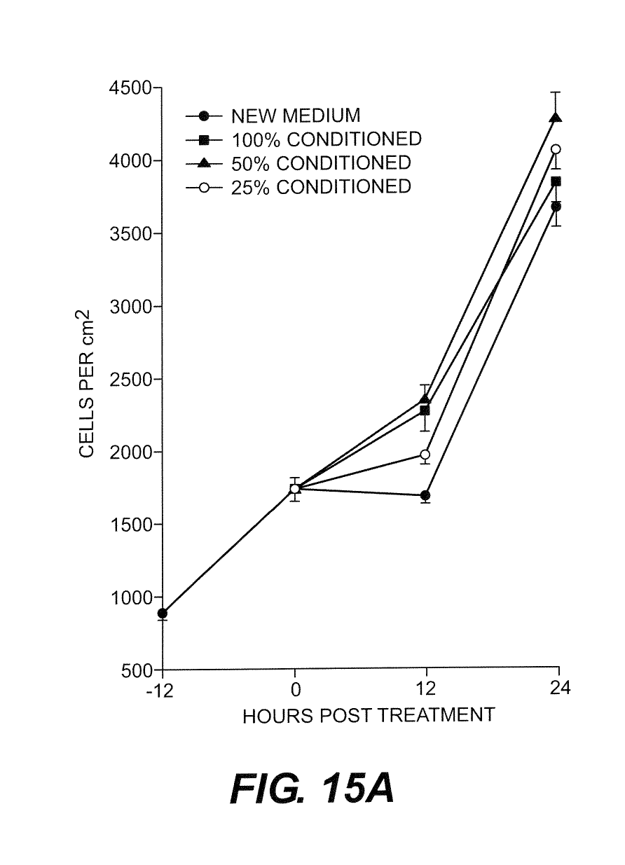

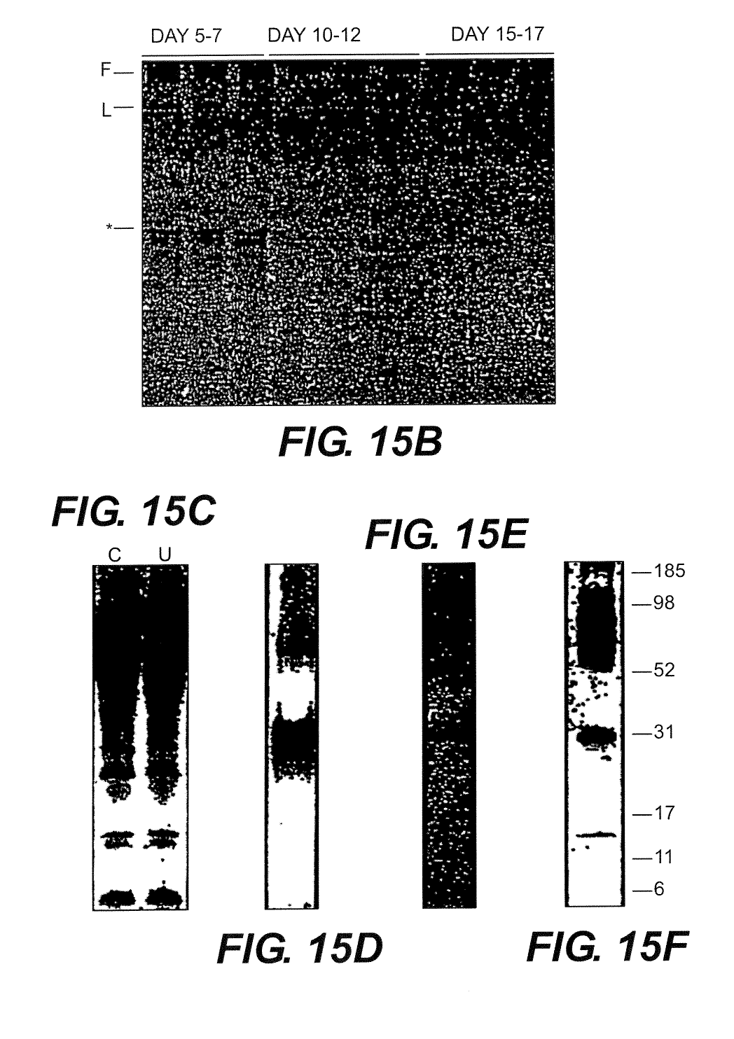

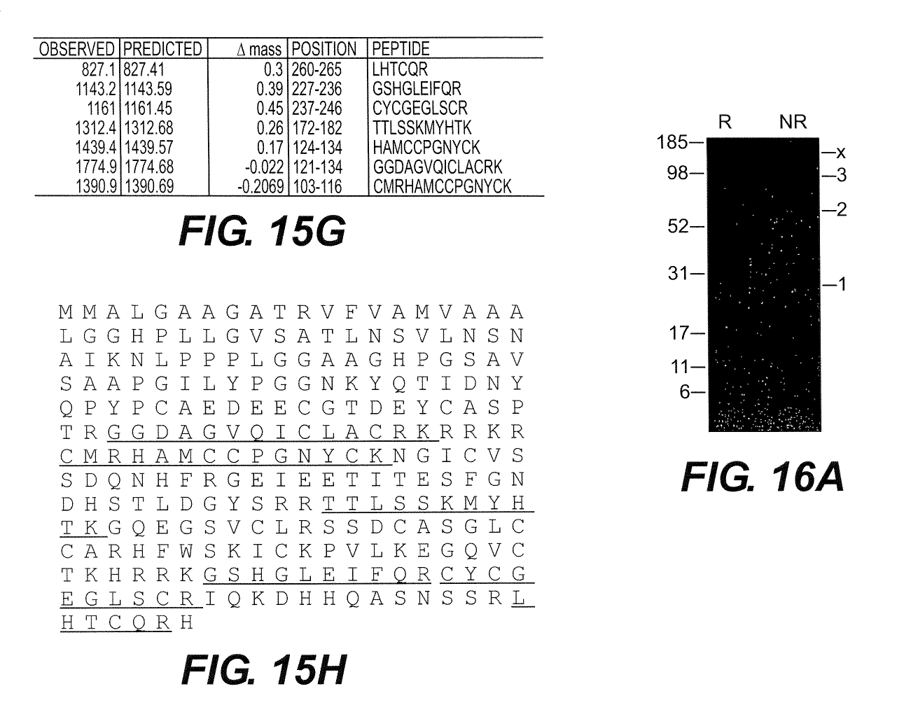

Initial studies with hMSCs (FIG. 15A) demonstrated that the growth of early log-phase cultures of hMSCs is arrested for approximately 12 hours after replacement of conditioned medium with fresh medium. By adding various proportions of conditioned medium from rapidly dividing hMSCs, the delay in proliferation was proportionately decreased. The results therefore suggested that the cultures of hMSCs must re-establish a critical concentration level of one or more secreted factors to re-enter cell cycle.

Analysis of Secreted Proteins by [³⁵S]-Methionine Labeling

To identify newly synthesized proteins in the medium hMSCs were plated at a density of 100 cells per cm² and allowed to grow in medium containing 20% (v/v) FCS. Cells were labeled in the presence of 5 microcuries per milliliter of [³⁵S]-methionine for 48-hour periods between days 5 and 7, days 10 and 12 or days 15 and 17. The early log phase of growth at days 5 to 7 was accompanied by the largest incorporation of radiolabel and the largest secretion of labeled protein (FIG. 15B). The most abundant labeled proteins were 185 kDa and 100 kDa (FIG. 15B). Western blotting and immunoprecipitation demonstrated that these proteins were fibronectin and laminin, respectively. An additional doublet of labeled protein was detected at 30 to 35 kDa (FIG. 15B), a region that contained relatively little unlabeled protein (FIG. 15C). The radiolabeled 30 to 35 kDa band (FIG. 15D) was eluted from the gel and examined by tryptic fingerprinting. Thirteen tryptic peptides were detected by surface-enhanced laser desorption/ionization mass spectrometry. The data were analyzed by the Pepmapper algorithm (Wilkins & Williams 1997) with appropriate settings for detection of oxidized methionine and acryl-cysteine modifications Seven of the thirteen peptides were identical within 0.5 kDa to tryptic peptides from Dkk-1 (FIGS. 15G and 15H). The remaining six peptides corresponded to tryptic peptides from bovine prothrombin also detectable in the appropriate fraction of control media not conditioned by hMSCs.

A rabbit polyclonal antibody was produced against a peptide corresponding to a IS residue long sequence in the second cysteine rich domain of Dkk-1 and used to probe western blots of medium obtained from rapidly expanding hMSCs. A band of 30 kDa was clearly visible (FIG. 15E). Also, a small amount of Dkk-1 was recovered from conditioned medium by immunoaffinity chromatography using the same antibody (FIG. 15F).

Expression of Recombinant Dkk-1 in E. coli

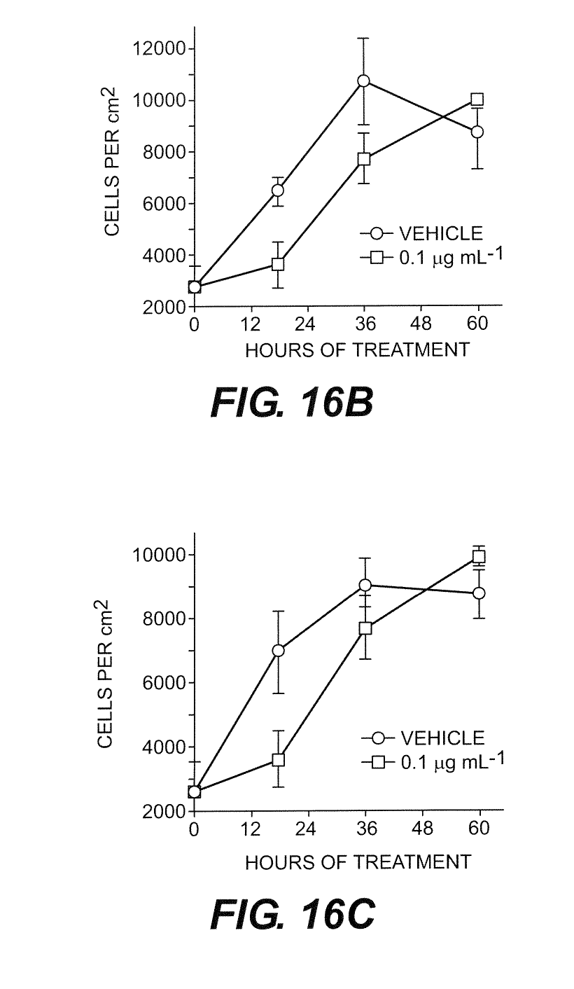

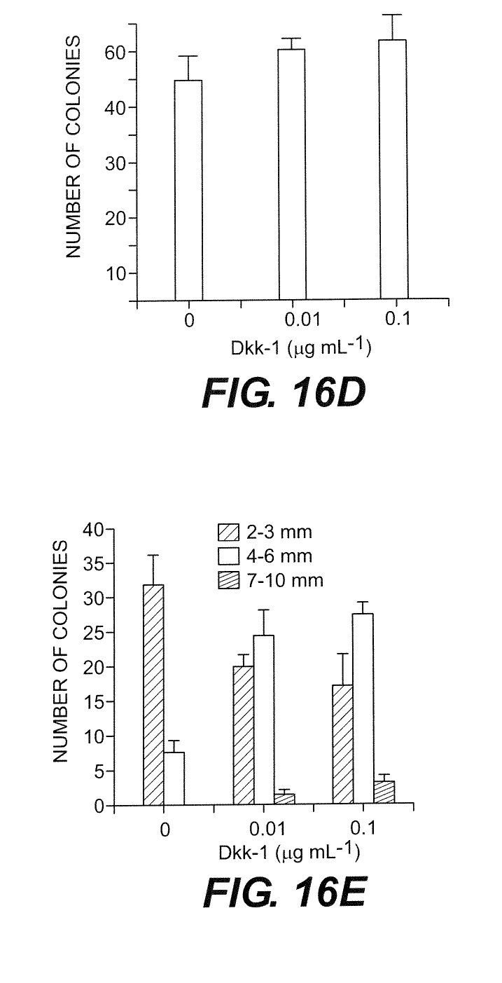

To prepare recombinant Dkk-1, the cDNA encoding the entire coding region of was cloned into the bacterial expression vector, pET 16b. The clone was constructed to encode an in-frame hexahistidine tag at the amino-terminus for protein purification. Recombinant Dkk-1 was recovered in insoluble inclusion bodies from the bacteria. The protein was solubilized, refolded and purified. The yield of protein was relatively low at approximately 100 micrograms of soluble protein per liter of culture. Assays by SDS-PAGE under reducing and non-reducing conditions indicated that about 60% of the protein had concatamerized through inter-molecular disulfide bond formation (FIG. 16A). Circular dichroism indicated that a significant fraction of the protein was alpha helical, a conclusion that agreed with the theoretical prediction of the secondary structure by the PHDsec algorithm (Rost et al., J. Mol. Biol. 270:471-480, 1997).

Effect of Recombinant Dkk-1 on hMSC Proliferation

To test the hypothesis that Dkk-1 increased proliferation of hMSCs, its effects on rate of growth were assayed. The hMSCs were plated at a density of 100 cells per cm² in 6 well plates (10 per cm² per well). After 4 days, when the cells were in early log phase of growth, the conditioned medium was removed and replaced with fresh medium containing either vehicle, 0.1 micrograms per milliliter Dkk-1 or 0.01 micrograms per milliliter Dkk-1. Fluorescence assays for nucleic acids indicated that the recombinant Dkk-1 reduced the lag phase and initially increased proliferation (FIG. 16A). It had no significant effect on proliferation as the cells approached the stationary phase of growth. The effect of Dkk-1 persisted for 30 hours at 0.1 micrograms per milliliter (FIG. 16B) whereas the effects of Dkk-1 were only significant for about 15 hours when tenfold less Dkk-1 was added (FIG. 16C), suggesting that the molecule had a short half-life.

To test the effect of recombinant Dkk-1 on the colony-forming potential of hMSCs, 100 hMSCs were plated onto a 176 cm² tissue culture dish and allowed to form colonies in the absence or presence of Dkk-1 in medium supplemented with 10% (v/v) fetal calf serum instead of the optimal concentration of 20%. After 2.5 weeks, the recombinant Dkk-1 increased colony size (FIG. 16E). However, there was no significant effect on colony number (FIG. 16D). The effects of Dkk-1 appeared to be biphasic in that concentrations as high as 0.5 micrograms per milliliter failed to increase the rate of proliferation and reduced both the colony size and number.

RT-PCR Assays for Dkk-1 and LRP-6

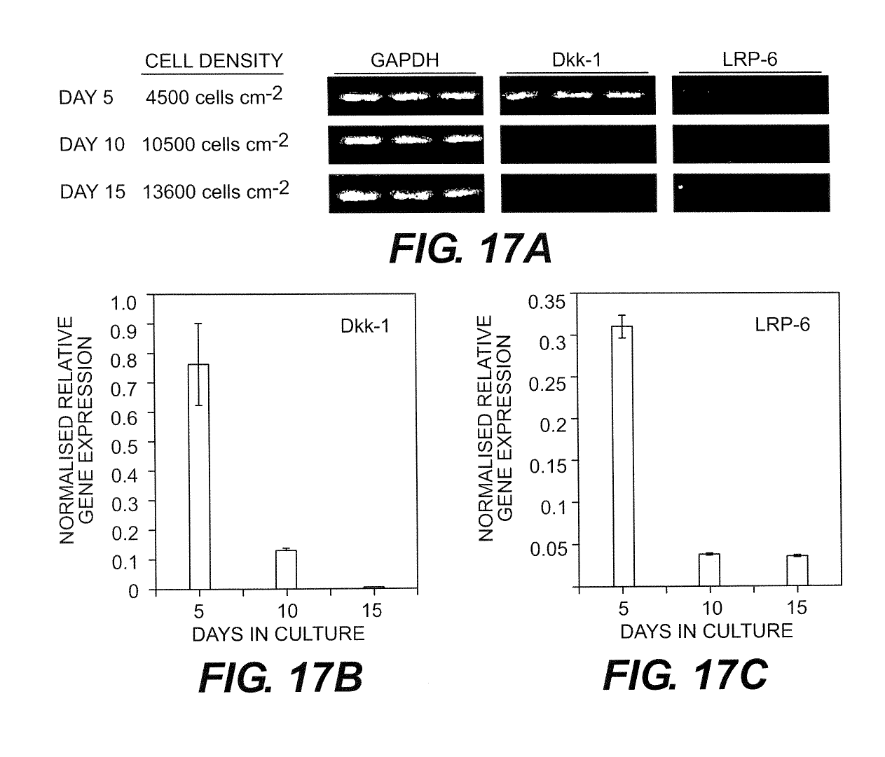

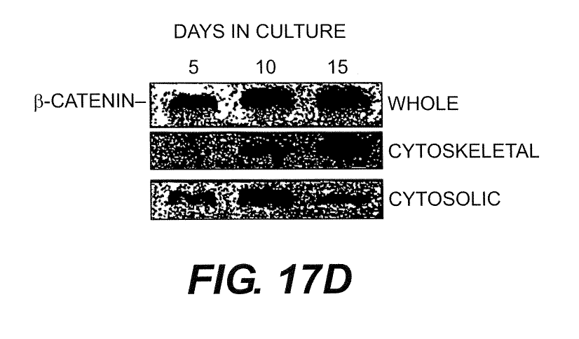

To investigate the mRNA profiles of Dkk-1 and its receptors and more closely, a previously described quantitative RT-PCR and ELISA-based assay was employed (Gregory et al., Anal. Biochem. 296:114-121, 2001). The level of Dkk-1 mRNA was highest after 5 days in culture and not detectable at 10 and 15 days (FIG. 17A). Expression of one of the Dkk-1 receptors, LRP-6, paralleled expression of Dkk-1 with levels falling as hMSCs become confluent (FIG. 17A). Multiple attempts to amplify LRP-5 using different primers were unsuccessful. Data obtained with a more sensitive digoxygenin (DIG)-labeled RT-PCR assay also indicated that Dkk-1 and LRP-6 transcription decreased over time in culture (FIGS. 17B and 17C).

To explore the observations further, beta-catenin levels were assayed based on the assumption that Dkk-1 expression early in culture would inhibit the canonical Wnt pathway leading to a destabilization of beta-catenin. As expected, western blotting demonstrated that the steady-state level of beta-catenin was lower in early log phase cultures than in late log or stationary phase cultures (FIG. 17D). Also, the beta-catenin molecules in the stationary phase were extensively redistributed from the cytoplasmic pool to the detergent-insoluble cytoskeletal fraction (FIG. 17D), suggesting that beta-catenin contributed to the formation of actin-associated intracellular adherens junctions.

Microarray analyses of mRNA levels from hMSCs in culture also confirmed that several components of the Wnt signaling pathway were expressed (FIGS. 18A and 18B). As expected, the signal intensity for Dkk-1 mRNA was high in early log phase of growth and decreased over 2-fold between 5 and 15 days in culture. There were only minor changes in other components of the Wnt pathway, including Dkk-3, LRP-5, LRP-6, Wnt-5a, a series of catenins, 4 frizzleds, frizzled-regulated protein, disheveled and three forms of GSK. Similarly, a series of cadherins were expressed but there were no significant changes with time in culture. As expected, there were several minor inconsistencies between the micro-array and RT-PCR data.

Recombinant Dkk-1 decreases the concentration and re-distributes beta-catenin to cell-to-cell contacts

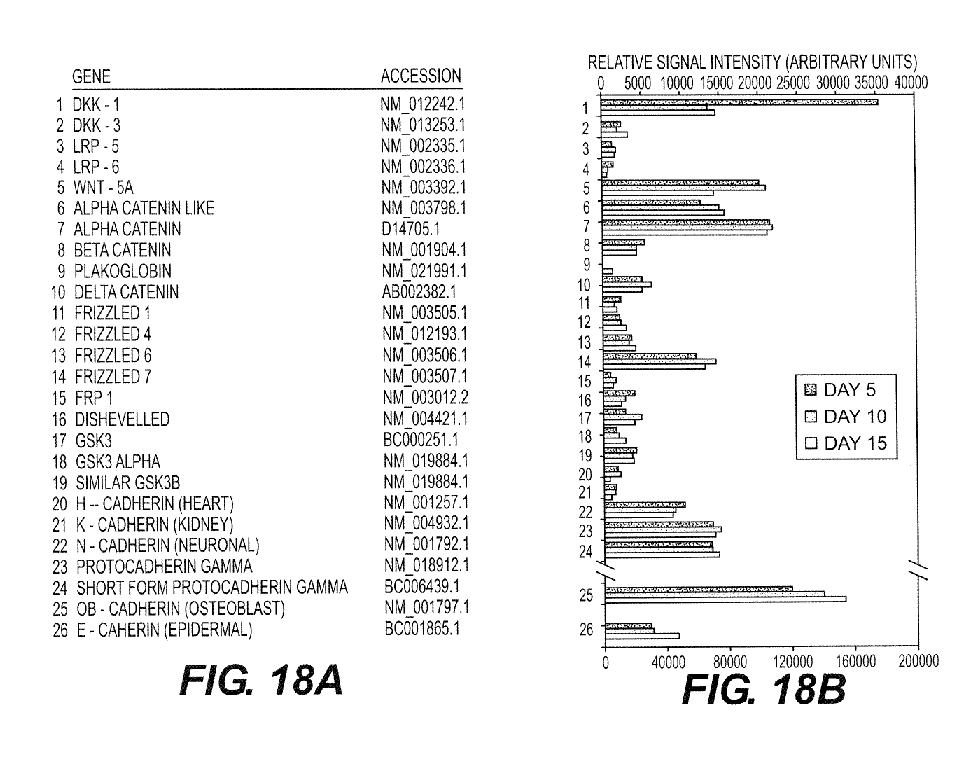

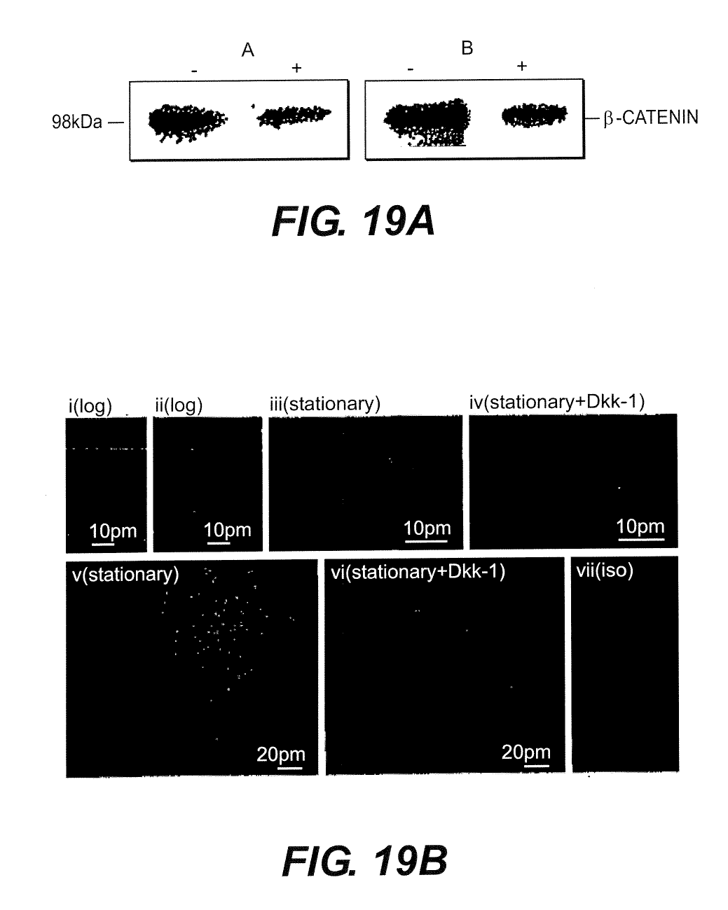

In further experiments, the effect of recombinant Dkk-1 on beta-catenin levels in hMSCs were investigated. As expected, treatment of stationary phase cultures of hMSCs with 0.1 micrograms per milliliter recombinant Dkk-1 reduced the levels of beta-catenin (FIG. 19A).

To examine effects of the recombinant Dkk-1 on the cellular distribution of beta-catenin, monolayers were fixed with paraformaldehyde at the early log phase (6 days) or stationary phase (15 days) of growth, and sections of the dish were immuno-stained for beta-catenin. In untreated early log phase cultures, beta-catenin was distributed throughout the cytoplasm and the plasma membrane at areas of cell-cell contact (FIGS. 19Bi and 19Bii). In many instances of cell-cell contact, there appeared to be a gradient of beta-catenin distribution throughout the cytoplasm with most concentration proximal to the contact site (FIG. 19Bi). In stationary cultures, the distribution of beta-catenin was similar but the concentration at cell contacts was more apparent (FIGS. 19B iii and 19Bv). As expected, addition of medium containing 0.1 micrograms per milliliter Dkk-1, produced a clearance of the cytoplasmic pool of beta-catenin resulting in a more pronounced localization at sites of intercellular contact (FIGS. 19Biv and 19Bvi). Low power images confirmed that the effect of Dkk-1 was present throughout the monolayer (FIGS. 19Bv and 19Bvi). The staining was specific for beta-catenin since extended exposure of the control slides with an appropriate concentration of isotype control did not give a fluorescent signal (FIG. 19B vii).

Dkk-1 Expression is Concomitant with Cell Cycle Activity

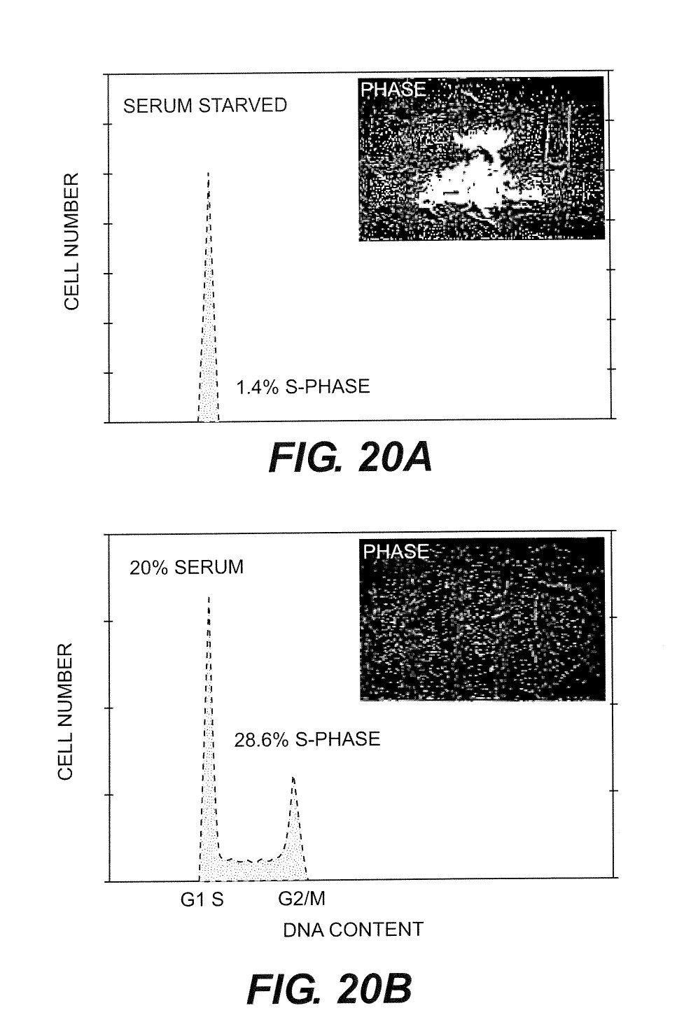

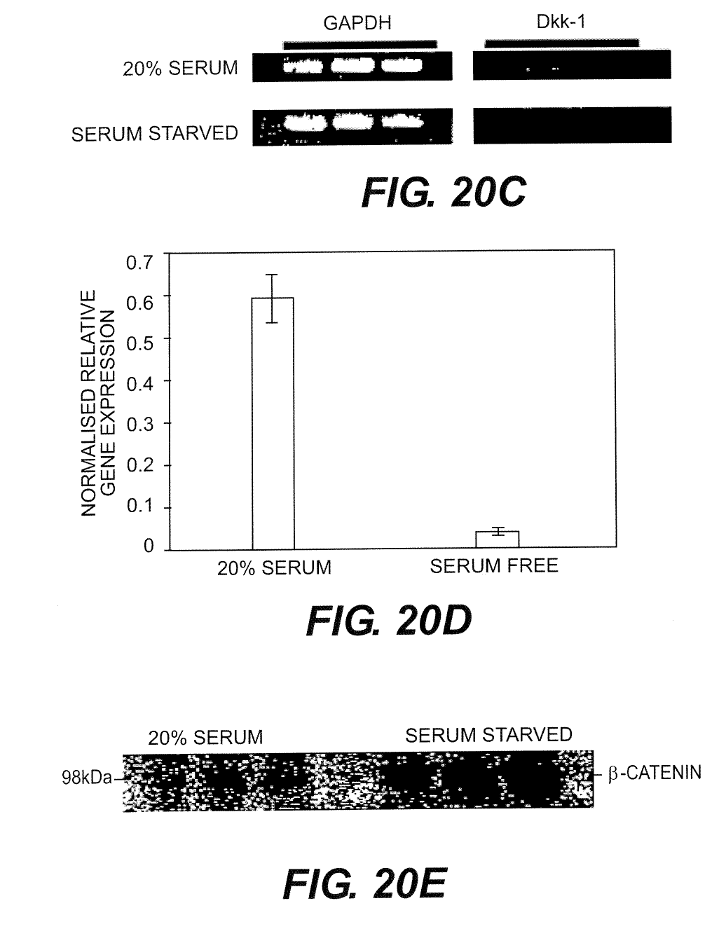

Since Dkk-1 expression was highest in hMSCs during the early log phase of growth, the hypothesis that expression of Dkk-1 would decrease if the cells were growth arrested by serum starvation was tested (FIGS. 20A and 20B). Hybridization ELISA of RT-PCR products indicated that Dkk-1, but not GAPDH levels, were significantly reduced under conditions that inhibit division (FIGS. 20C and 20D). In addition, beta-catenin levels were increased in the growth arrested hMSCs (FIG. 20E), possibly in response to the reduction of Dkk-1 synthesis.

Effect of anti Dkk-1 Antibodies on hMSCs and Malignant Cell Lines

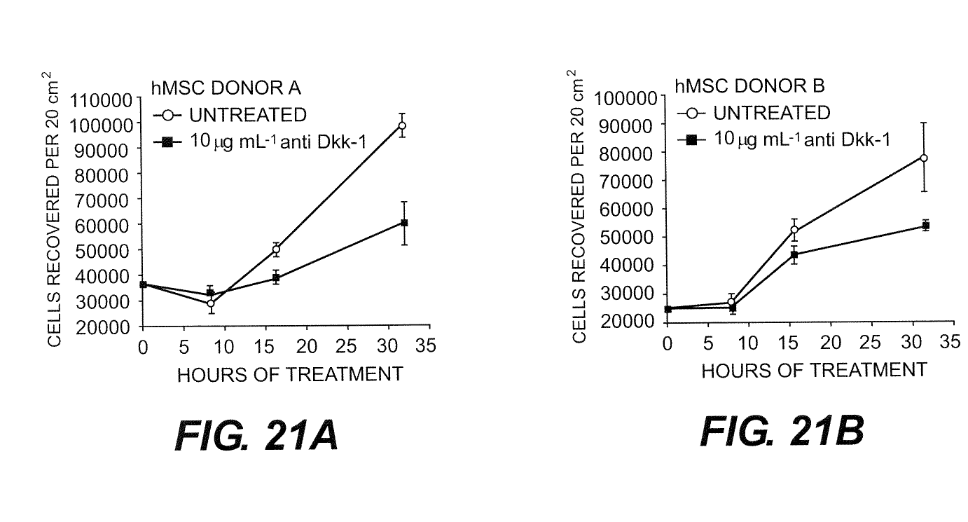

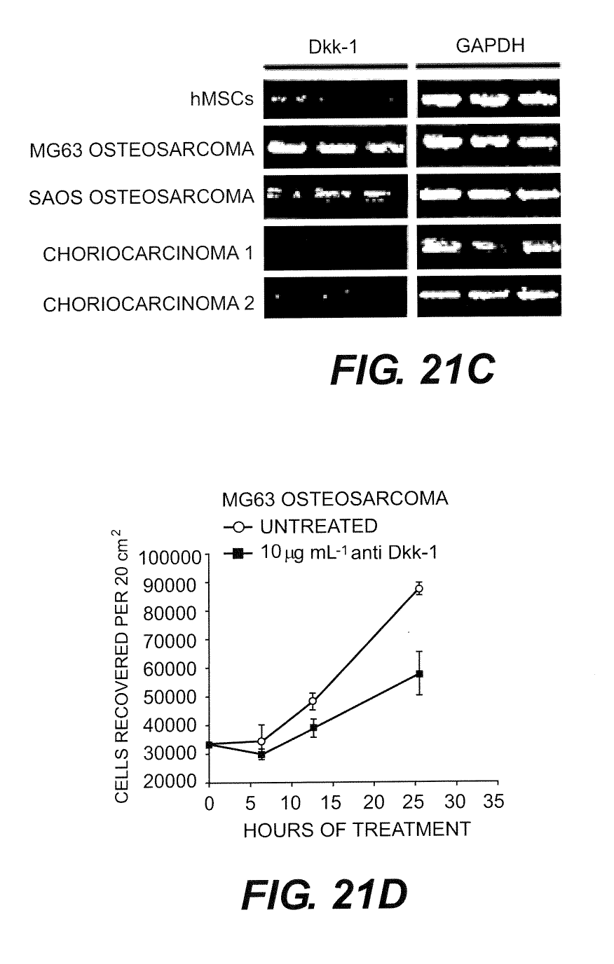

The antiserum to the synthetic peptide from Dkk-1 (FIG. 15E) was added to the medium from 5-day cultures of hMSCs. As indicated in FIGS. 21A and 21B, the antiserum slowed the proliferation of the cells obtained from two different donors. Addition of higher concentrations of the antiserum (50 micrograms per milliliter) had no effect on stationary cultures of hMSCs. Therefore the effects were specific for rapidly proliferating hMSCs.

Three lines of human malignant cells were assayed for expression of Dkk-1 by RT-PCR. mRNA for Dkk-1 was present in both osteosarcoma lines tested and at much lower levels in one of the two choriocarcinoma lines (FIG. 21C). Addition of anti Dkk-1 antibodies to the medium slowed the growth of the one osteosarcoma cell line tested (FIG. 21D).

### Example 4

### Removal of Internalized Calf Serum

In the present experiment, FCS contamination from hMSCs was minimized while maintaining the proliferation capacity necessary to generate clinically-relevant numbers of cells. First a sensitive, quantitative assay to measure FCS was developed. Several growth media were tested for their ability to remove FCS contamination from hMSCs.

The Materials and Methods used in the experiments presented in this Example are now described.

Preparation of JFCS

One hundred milliliters of a 14 milligrams per nanograms per milliliter solution of FCS (Atlanta Biologicals, Norcross, Ga.) was prepared for fluorescent labeling by diafiltration into 20 volumes of 20 millimolar NaCO₃NaHCO₃ buffer (pH 9.5) using a Millipore tangential flow filtration system fitted with 150 cm² PVDF 5 kDa filters. The sample was then added to 0.5 grams of FITC (Sigma Aldrich Incorporated, St. Louis, Mo.) dissolved in 5 milliliters DMSO (Fisher Lifesciences, Pittsburgh, Pa.). After vigorous shaking for 10 minutes, the reaction was incubated at 4° C. for 16 hr and then stopped by addition of 0.1 volumes of 1 molar Tris HCl buffer (pH 8.0) (Sigma Aldrich Incorporated; St. Louis, Mo.) to a final concentration of 100 millimolar. The sample was cleared by centrifugation at 6,000 g then filtered through a 0.22 micron Durapore membrane (Millipore Corporation, Bedford, Mass.).

Unincorporated label was removed by diafiltration against approximately 50 volumes of 1× phosphate buffered saline (Fisher Lifesciences). Throughout the diafiltration, samples (300 microliters) were taken intermittently to monitor fluorescence and 30 micrograms of the final sample was analyzed by 4 to 20% SDS PAGE (Novex System, Invitrogen Corporation, Carlsbad, Calif.) under reducing conditions followed by fluorescent imaging of the gel (Typhoon Imaging System, Amersham Pharmacia, Piscataway, N.J.). Whole protein concentration was quantified by Bradford assay (BioRad Laboratories, Hercules, Calif.). Finally, each batch of fFCS was adjusted to its original protein concentration by diafiltration.

Preparation of Human Serum

Five hundred milliliters of whole blood was taken from consenting donors who had previously donated bone marrow for preparation of hMSCs.

The blood was recovered into 600 milliliter blood bags (Baxter Fenwall, Deerfield, Ill.) in the absence of anti-coagulants and allowed to clot for 4 hours at room temperature. The serum (100 to 150 milliliters) was aspirated from the clot and centrifuged at 500 g for 20 minutes. The supernatant was then centrifuged for a further 20 minutes at 2,000 g. The cleared serum was incubated at 56° C. for 20 minutes to deactivate complement followed by storage at −80° C. Medium containing the human serum was filtered through a 0.22 micron membrane before use.

Tissue Culture

hMSCs were prepared and grown as previously described above. Briefly, for FCS uptake experiments, cells were seeded into 10 cm² plates (Costar; Fisher Lifesciences, Pittsburgh, Pa.) at 100 cells per cm² and allowed to grow in complete medium containing 20% FCS for 4 days before replacement with medium containing 20% (v/v) fFCS. The cell culture was incubated in the presence of fFCS for 24 hours followed by three brief washes with phosphate buffered saline. Cells were visualized by phase contrast and epifluorescence microscopy (Nikon Eclipse TE200) and documented by digital imaging. hMSCs were also examined by deconvolution epifluorescence microscopy with a Leica DMRXA microscope equipped with an automated x, y, z stage and CCD camera (Sensicam, Intelligent Imaging Innovations, Denver, Colo.).

Images taken at 1.0 micron intervals were deconvoluted using commercial software (Slidebook software, Intelligent Imaging Innovations, Denver, Colo.). The removal of fFCS from the cells was optimized by incubation in alpha-MEM containing 100 units per milliliter penicillin, 100 micrograms per milliliter streptomycin and 2 millimolar L-glutamine (Fisher Lifesciences, Pittsburgh, Pa.) alone or in the presence of 20% (v/v) human serum (Fisher Scientific, Pittsburgh, Pa.) or 10% (v/v) human serum with 10 nanograms per milliliter EGF (Sigma Aldrich, St. Louis, Mo.) and bFGF 10 nanograms per milliliter (Sigma Aldrich, St. Louis, Mo.). Unlabeled 20% (v/v) FCS was used as a positive control. In some experiments, cells were incubated in commercially available human serum (Fisher Lifesciences, Pittsburgh, Pa.).

To test serum-free media, hMSCs were plated at 100 cells per cm² in 12-well plates and tested in a 3-dimensional combinatorial assay. The baseline medium in all samples was alpha-MEM. In each experiment, a stack of three 12-well plates was used. In the first experiment, 10 nanograms per milliliter EGF and 10 nanograms per milliliter bFGF was added to all 36 wells. Transferrin at 3, 6 or 9 micrograms per milliliter was added to wells in the y-axis; 2, 4, 6, or 8 micrograms per milliliter of linoleic acid was added in the x-axis, and 2, 4 or 6 micrograms per milliliter of human serum albumin (HSA) in the z-axis.

Few viable cells were seen by microscopy after 12 to 14 days. In a second experiment, 2 milligrams per milliliter of HSA were added to the alpha-MEM in all 36 wells and the z-axis varied to contain (a) 10 nanograms per milliliter insulin-like growth factor; (b) 10 nanograms per milliliter each of IGF, EGF and bFGF; and (c) 10 nanograms per milliliter EGF, 10 nanograms per milliliter bFGF, and 5 nanograms per milliliter platelet-drived growth factor-BB. Few viable cells were seen after 14 days.

In a third experiment, the z-axis was varied to contain 5, 7.5 or 10 nanograms per milliliter of stem cell factor. Again, few viable cells were seen at 14 days. All reagents were from Sigma except stem cell factor was from Chemicon (Temecula, Calif.).

Fluorescence Analysis

Cells from two wells of a 6-well plate (9.6 cm² each) were recovered by trypsinization at 37° C. for 5 minutes with 0.25% (w/v) trypsin and 1 millimolar EDTA (Fisher Lifesciences, Pittsburgh, Pa.), counted by hemacytometer, and suspended in distilled H₂O. The suspended cells were lysed by three freeze-thaw cycles at −80° C. and 37° C. respectively. Three aliquots of 150 microliters were transferred to individual wells of an opaque-walled microtiter plate (Costar; Fisher Lifesciences, Pittsburgh, Pa.). A fluorescence reader (Power Wave HT; FLx800; Biotek Instruments, Winooski, Vt.) set to 485 nanometers excitation and 530 nanometers emission and was employed to assay the fluorescence.

ATP Measurements

Cells were recovered by trypsinization, counted by hemacytometer and suspended in distilled H₂O at a concentration of 2 million cells per milliliter. Cells were lysed by incubation at 95° C. for 5 minutes followed by recovery of the soluble fraction of the lysate by centrifugation at 12,000 g for 15 minutes. A colorimetric assay kit was employed to quantify the concentration of ATP in the extract (Sigma Aldrich, St. Louis, Mo.). Three readings were taken on 150 microliter aliquots of the extract.

Flow Cytometry

Cells were recovered by trypsinization, suspended in PBS and phenotyped based on forward and side scatter using a flow cytometer (Epics XL; Beckman Coulter, Brea, Calif.).

Microarray Analysis

hMSCs from two separate donors were plated at 50 cells per cm² and cultured in standard medium containing 20% FCS for 7 days with a change of medium on day 4. The cultures were then incubated for 3 days either in the standard medium or in AHS⁺. Microarray assays were performed according to the manufacturer's recommendations (Affymetrix GeneChip Expression Analysis Technical Manual; Affymetrix, Santa Clara, Calif.).

In brief, 8 micrograms of total RNA was used to synthesize double-stranded DNA (Superscript Choice System; Life Technologies, Rockville, Md.). The DNA was purified by phenol/chloroform and concentrated by ethanol precipitation. In vitro transcription of biotin-labeled cRNA was performed using a commercial kit (BioArray HighYield RNA Transcription Labeling Kit; Enzo Diagnostics, Farmingdale, N.Y.) and labeled cRNA was cleaned (RNeasy Mini Kit; Qiagen, Valencia, Calif.). Twenty-five micrograms of labeled cRNA was fragmented to 50 to 200 nucleotides and hybridized for 16 hours at 45° C. to an array (HG-U133A), which contains approximately 22,200 human genes.

After washing, the array was probed with streptavidin-phycoerythrin (Molecular Probes, Eugene, Oreg.), amplified by biotinylated anti-streptavidin (Vector Laboratories, Burlingame, Calif.) and re-probed with streptavidin-phycoerythrin. The chip was then scanned (Hewlett-Packard GeneArray Scanner). The raw data were analyzed using Affymetrix MicroArray Suite v5.0 and Affymetrix Data Mining Tool v3.0. Signal intensities of all probe sets were scaled to the target value of 2,500. The Pearson correlation coefficient (r²) was calculated from the linear regression of the data (Microsoft Excel).

Differentiation into Bone and Adipose

For osteogenic differentiation, confluent monolayers were incubated in medium supplemented with 10⁻⁸ molar dexamethasone, 0.2 molar ascorbic acid and 10 millimolar beta-glycerol phosphate. For adipogenic differentiation, the medium was 0.5 millimolar hydrocortisone, 0.5 millimolar isobutyl-methyl-xanthine and 60 micromolar indomethacin (Sigma Aldrich, St. Louis, Mo.).

After 3 weeks, the cells were washed with PBS and fixed in 4% (v/v) paraformaldehyde (USB Corporation, Cleveland, Ohio) for 5 minutes. Bone mineral was stained using 40 millimolar Alizarin Red (pH 4.1) (Sigma Aldrich, St. Louis, Mo.) and fat droplets were stained using 0.1% (v/v) Oil Red O in 60% (v/v) isopropanol. Plates were washed extensively with deionised water (osteogenic staining) or PBS (adipogenic staining) prior to phase microscopy.

The Results of the experiments presented in this Example are now described.

One hundred milliliters of FCS (14 milligrams per milliliter; Atlanta Biologicals; Norcross, Ga.) was covalently labeled by reaction with 0.5 grams of fluorescein-isothio-cyanate (FITC; Sigma Aldrich, St. Louis, Mo.) in 5 milliliters DMSO. After 16 hours at 4° C., the FITC-labeled FCS (fFCS) was extensively diafiltered. Assays of the fFCS by SDS-PAGE and fluorescence demonstrated efficient labeling of a wide range of serum components.

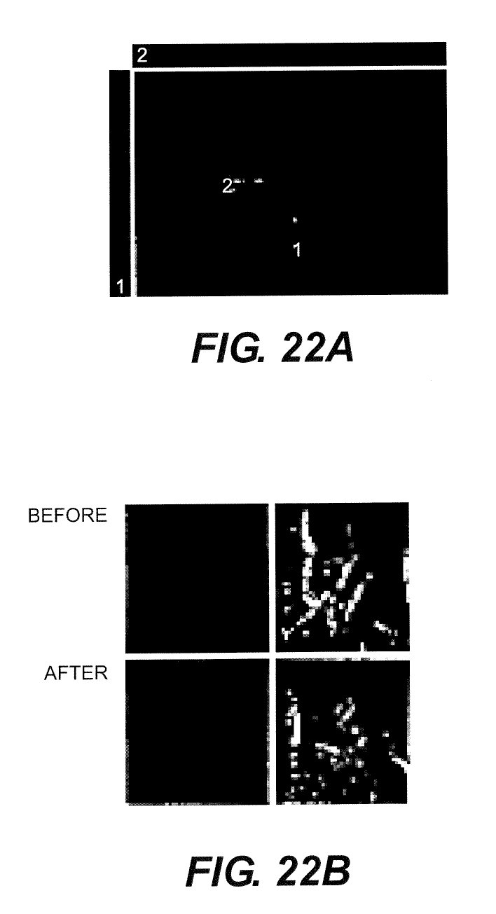

To evaluate the FCS contamination, monolayers of hMSCs were plated at 50 or 500 cells/cm² and expanded for 4 days in complete medium containing 20% fFCS. The medium was then replaced by fresh medium containing 20% fFCS and the cultures were incubated for 2 more days. Deconvolution microscopy demonstrated that some of the fFCS was internalized (FIG. 22A). Fluorescence assays on cell lysates indicated that after trypsinization and extensive washing with a variety of buffers, each cell on average was still associated with 85 to 300 picograms of fFCS. Therefore, a protocol was designed to remove the internalized FCS.

Numerous FCS-free media preparations were tested in assays for rates of propagation, viability and morphology. None of the conditions tested were as effective as autologous human serum supplemented with 10 nanograms per milliliter epidermal growth factor (EGF) and 10 nanograms per milliliter basic fibroblast growth factor (bFGF), hereafter called AHS⁺. AHS⁺ from 6 separate donors was as effective as FCS in supporting cell growth. Surprisingly, a commercial human serum gave poor cellular yields, with notable cell death and phenotypic deterioration.

Because of the limited supply of autologous human serum, a protocol was developed in which the cultures were first expanded in medium containing FCS and then transferred to AHS⁺. hMSCs were plated at 50 or 500 cells per cm², expanded in medium containing 20% FCS for 4 days, and labeled by incubation for two days in medium containing 20% fPCS.

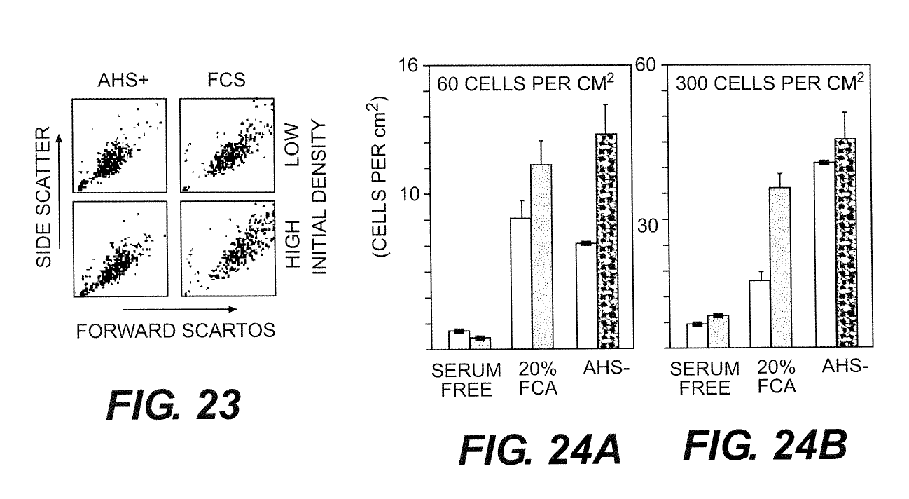

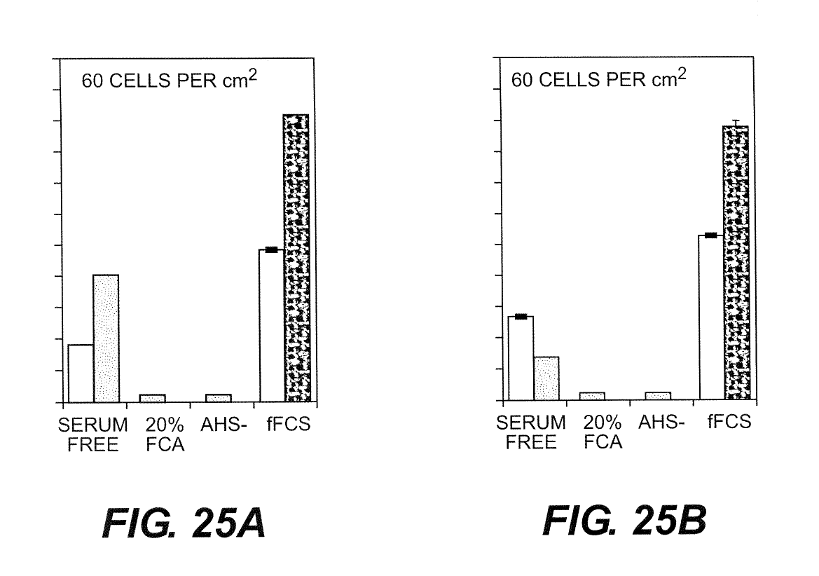

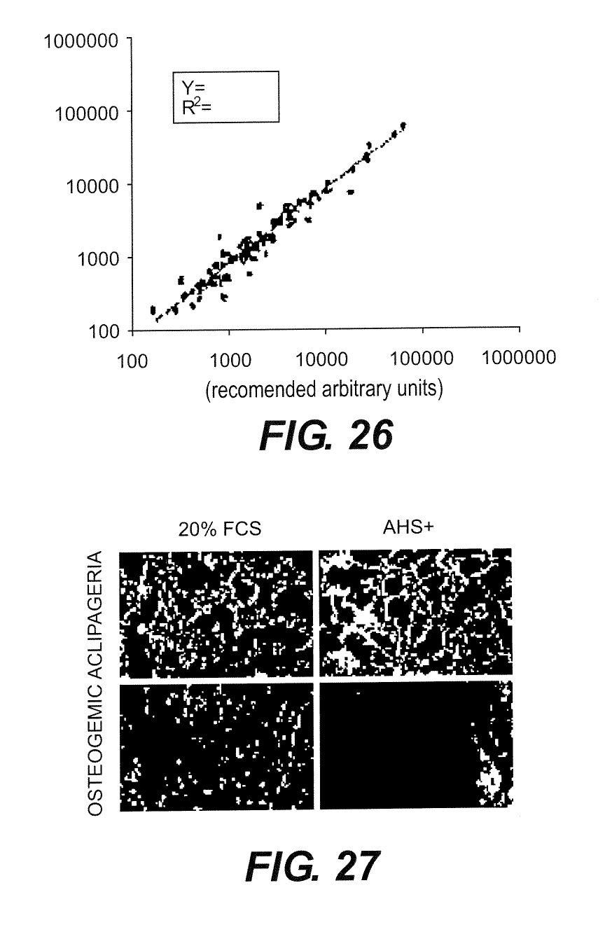

Triplicate samples for two donors were then incubated for 2 or 4 days in one of the following: (i) serum free medium, (ii) medium containing 20% unlabeled FCS, or (iii) AHS⁺ (FIGS. 24A-25B). The medium was replaced with fresh medium at 6 hours, 2 days, and 4 days. The cellular yield with AHS⁺ was better than or comparable to incubation in 20% FCS. In contrast, the yield was low in serum-free medium compared to cultures with FCS. The cultures grown in AHS⁺ had a higher content of cells that were lower in forward scatter and side scatter of light (FIG. 23), indicating that they were enriched for rapidly self-renewing early progenitor cells^{5,6}. Microarrays (Affymetrix, Santa Clara, Calif.) were used to assay mRNA levels in cells incubated with AHS⁺ versus those grown in FCS. Comparison of 113 genes randomly selected from a total of 11,131 gave a linear correlation coefficient of 0.9776 (FIG. 26). hMSCs expanded in AHS⁺ for 10 days differentiated into adipocytes and osteoblasts as readily as hMSCs expanded in FCS (FIG. 27).

Since hMSCs grown in FCS retained 85 to 300 picograms of fFCS per cell after trypsinization and washing, a common therapeutic dosage of 100 million hMSCs would be associated with 7 to 30 milligrams of FCS. After incubation with AHS⁺ for 4 days with the protocol described here, the cells retained less than 10 nanograms per 100 million cells; therefore the reduction in fFCS was at least 99.9%. A similar protocol should be applicable to other cells that are cultured in FCS.

### Example 5

### Peptides of Dkk-1 Selectively Bind to RS Cells

The Materials and Methods used in the experiments presented in this Example are now described.

Cells were cultured according the methods described elsewhere herein.

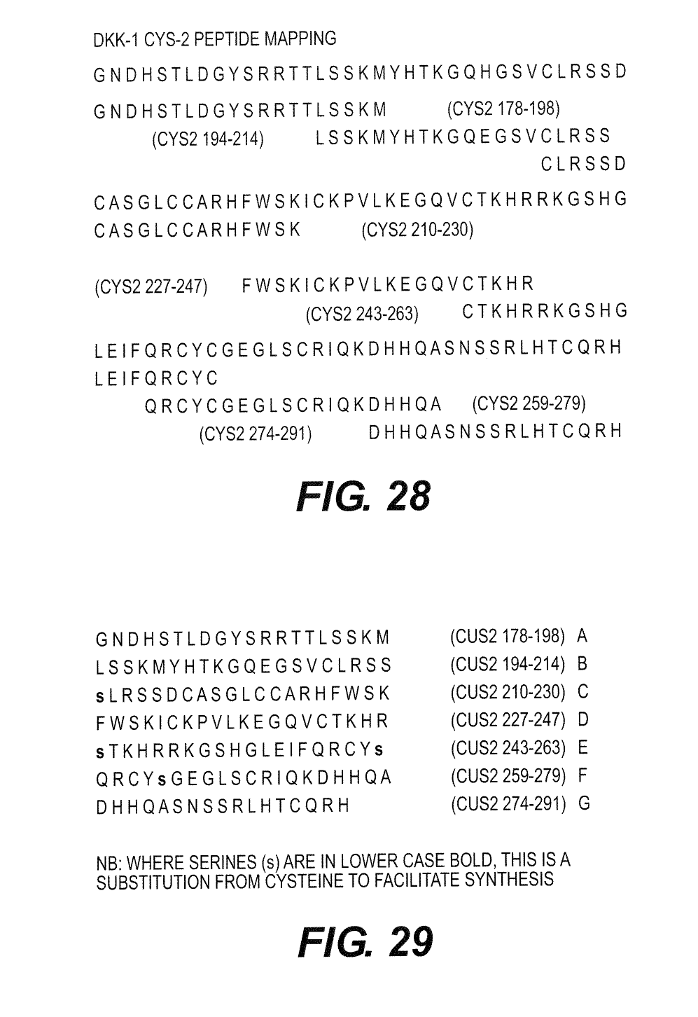

A series of peptides (SEQ ID NOS:11-17) were commercially synthesized from the LRP-6 binding site of Dkk-1 (SEQ ID NO:10). The LRP-6 binding site was mapped using cys-2 peptide mapping, depicted in FIG. 28. The amino acid sequence of the LRP-6 binding domain of Dkk-1 is as follows:

(SEQ ID NO:10)

GNDHSTLDGYSRRTTLSSKMYHTKGQEGSVCLRSSDCASGLCCARHFWSK
ICKPVLKEGQVCTKHRRKGSHGLEIFQRCYCGEGLSCRIQKDHHQASNSS
RLHTCQRH

Some cysteines in the peptides were substituted with serines to facilitate synthesis of the peptides. These substitutions are indicated by the lowercase “s” in the sequence. The synthesized peptide sequences were as follows (also depicted in FIG. 29):

GNDHSTLDGYSRRTTLSSKM
(Peptide A; SEQ ID NO:11)
LSSKMYHTKGQEGSVCLRSS
(Peptide B; SEQ ID NO:12)
sLRSSDCASGLCCARHFWSK
(Peptide C; SEQ ID NO:13)
FWSKICKPVLKEGQVCTKHR
(Peptide D; SEQ ID NO:14)
sTKHRRKGSHGLEIFQRCYs
(Peptide E; SEQ ID NO:15)
QRCYsGEGLSCRIQKDHHQA
(Peptide F; SEQ ID NO:16)
DHHQASNSSRLHTCQRH
(Peptide G; SEQ ID NO:17)

The peptides were then labeled with biotin for use with a commercially available streptavidin-biotin detection system. The streptavidin was linked to a fluorescent tag (Alexafluor 594, Molecular Probes, Eugene, Oreg.) so as to be easily detected by fluorescence microscopy. MSCs were incubated with one of the peptides and the streptavidin-biotin detection system as indicated by the manufacturer's instructions. Then the MSCs were observed under a fluorescence microscope. All of these methods are well-known in the art and are easily found throughout the literature.

The Results obtained by these experiments are now described.

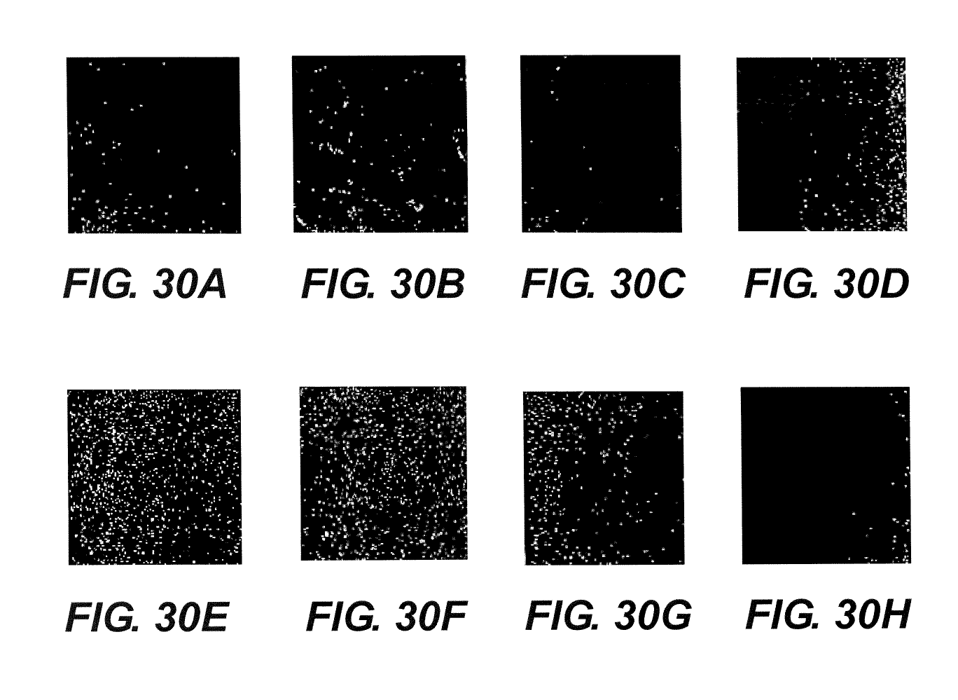

Upon examination, MSCs labeled with peptide B (SEQ ID NO:12) and peptide E (SEQ ID NO:15) were highly fluorescent, indicating that peptides B and E were tightly bound to what were later characterized as early progenitor cells, i.e., RS cells. The peptides did not bind to larger, more mature MSCs. Comparing FIGS. 30A-30G, only FIGS. 30B and 30E, corresponding to peptides having SEQ ID NO:11 and SEQ ID NO:15, respectively, fluoresced, and all of the cells were morphologically characterized as early progenitor cells.

### Example 6

### Serum Deprivation of MSCs Selects for Early Progenitor Cells

The Materials and Methods used in the experiments presented in this Example are now described.

Cell Culture

Human MSCs were prepared as described previously (Colter et al., 2001; Sekiya et al., 2002). In brief, nucleated cells were isolated with a density gradient (Ficoll-Paque; Pharmacia, Piscataway, N.J.) from 2 milliliters of human bone marrow aspirated from the iliac crests of normal volunteers under a protocol approved by an Institutional Review Board. All the nucleated cells (30 to 70 million) were plated in a 145 cm² dish in 20 milliliters of complete culture medium: alpha-MEM (GIBCO BRL, Rockville, Md.); 17% fetal bovine serum (FBS lot-selected for rapid growth of MSCs; Atlanta Biologicals, Norcross, Ga.); 100 units/milliliter penicillin; 100 micrograms/milliliter streptomycin; and 2 millimolar L-glutamine (GIBCO BRL, Rockville, Md.). After 24 hours at 37° C. in 5% CO₂, adherent cells were discarded and the adherent cells incubated in fresh medium for 4 days. The cells were lifted with 0.25% trypsin and 1 millimolar EDTA for 5 minutes at 37° C. and replated at 50 cells/cm² in an interconnecting system of culture flasks (6320 cm² Cell Factory, Nunc, Rochester, N.Y.). After 7 to 9 days, the cells were lifted with trypsin/EDTA, suspended at about 10⁶ cells/milliliter in 5% DMSO and 30% FCS in alpha-MEM and frozen in 1 milliliter aliquots in liquid nitrogen as Passage 1 cells. The vials of passage 1 cells were thawed, plated in a 60 cm² dish, incubated for 4 days, and lifted with trypsin/EDTA to recover viable cells. The cells were then plated in complete medium at 50 to 500 cells/cm², incubated for 4 to 7 days, and lifted with trypsin/EDTA to recover passage 2 cells. Later passage cells were obtained by re-plating the cells at 50 to 500 cells/cm², incubating them for 4 to 7 days, and recovering the cells with trypsin/EDTA.

To prepare serum derived (SD) cells and controls, passage 2 or later passage cells were plated at 50 to 500 cells/cm² in 15 centimeter diameter plates. One set of plates was washed with PBS and incubated with alpha-MEM without serum or growth factors to prepare SD cells. The second set was incubated with complete culture medium with FCS as a parallel control set. The medium was replaced every 4 days for 2 to 4 weeks. After serum deprivation, both control and SD cells were recovered by lifting with trypsin/EDTA and replated with complete culture medium with 17% FCS. Both controls and SD cultures were expanded in complete culture medium containing FCS.

Telomere Length Assay

To assay telomere length, the Day 0 sample was prepared by plating passage 2 hMSCs at 100 cells/cm² in a 15 centimeter diameter dish and incubating in complete medium for 5 days. The SD sample was prepared by incubation of the Day 0 sample in medium without FCS for 3 weeks and then replating all the surviving cells in a 15 centimeter diameter dish and incubating in complete medium for 5 days. The control sample was prepared by incubating the Day 0 sample in complete medium for 3 weeks, replating at 100 cells/cm² and then incubating in complete medium for 5 days. Genomic DNA was isolated from 1×10⁶ cells (MagNA Pure LC DNA Isolation Kit I; Roche Molecular Biochemicals, Switzerland) and telomere length was assayed with a commercial kit (Telo Tagg; Roche Molecular Biochemicals, Switzerland). In brief, 10 micrograms of genomic DNA was digested with Rsa 1 and Southern blotted onto a nylon membrane. Telomere lengths were determined using chemiluminescent assay to detect DIG labeled probe.

Western Blot Analysis

Cells were prepared as for the assays of telomere length and lysed in buffer (Lysis Buffer; Roche Molecular Biochemicals, Switzerland) supplemented with protease inhibitor cocktail (Sigma Biochemicals, St. Louis, Mo.) and protein was assayed (Micro BCA Kit; Pierce Biotechnology Inc., Rockford, Ill.). The cell lysate (50 to 100 micrograms of protein) was fractionated by SDS-polyacrylamide gel electrophoresis (Novex 12% gels, Invitrogen, Carlsbad, Calif.). The sample was transferred to a filter (Immobilon P; Millipore, Bedford, Mass.) by electro-blotting (Iminunoblotting Apparatus; Invitrogen, Carlsbad, Calif.). The filter was blocked for 30 minutes with PBS containing 5% nonfat dry milk and 0.1% Tween 20, and then incubated for 1 hour with primary antibody. For detection of p₂₁WAF1, the filter was incubated with 1:500 dilution of anti-p21 antibody (Pharmingen, San Diego, Calif.). p53 was detected by incubating with a monoclonal antibody (DO-1; Pharmingen, San Diego, Calif.). The filter was washed four times for 15 minutes each with PBS containing 0.1% Tween 20. Bound primary antibody was detected by incubating for 1 hour with horseradish peroxidase goat anti-mouse IgG (Pharmingen, San Diego, Calif.) diluted 1:10,000 in PBS containing 5% non-fat dry milk. The filter was washed with PBS containing 0.1% Tween 20 and developed using a chemiluminescence assay (West-Femto Detection Kit; Pierce Biotechnology Inc, Rockford, Ill.).

RT-PCR Analysis

RNA was isolated from 0.5×10⁶ cells (RNAeasy RNA Isolation Kit; Qiagen Inc., Valencia, Calif.) and 50 picograms of RNA was used to perform one step RT-PCR (Titan One Step RT-PCR Kit; Roche Biochemical, Switzerland). Five microliters of the product was loaded for agarose gel electophoresis. The following primer sets were used:

| | | |
|---|---|---|
| Gene | Forward Primer | Reverse Primer |

| | | |
|---|---|---|
| Oct-4 | 5′-cccccgccgtatgagttctg | 5′-tgtgttcccaattccttccttag |
| | (SEQ ID NO: 18) | (SEQ ID NO: 19) |
| hTERT | 5′-cgctggtggcccagtgcctg | 5′-ctcgcacccggggctggcag |
| | (SEQ ID NO:20) | (SEQ ID NO:21) |
| OCT-4: | 5′-cgctccggcccacaaatctc | 5′-ccgcacgacaaccgcaccat |
| | (SEQ ID NO:22) | (SEQ ID NO:23) |
| ODC | 5′-ccgcacgacaaccgcaccat | 5′-cgctccggcccacaaatctc |
| antizyme | (SEQ ID NO:24) | (SEQ ID NO:25) |
| ATF-5 | 5′-aaggagctggaacagatggaagac | 5′-ttgtaaacctcgatgagcaggtcc |
| | (SEQ ID NO:26) | (SEQ ID NO:27) |
| FGF2 | 5′-gtgtgctaaccgttacctggctat | 5′-aggtaagcttcactgggtaacagc |
| | (SEQ ID NO:28) | (SEQ ID NO:29) |
| FGF2R | 5′-tgtgctaaccgttacctggctatg | 5′-aggtaagcttcactgggtaacagc |
| | (SEQ ID NO:30) | (SEQ ID NO:31) |
| GST | 5′-tgggaagaacaagatcacccagag | 5′-gttgtccaggtagctcttccaagt |
| | (SEQ ID NO:32) | (SEQ ID NO:33) |
| KAP1 | 5′-acccaaccttcagatcaactcctg | 5′-ccggttgagaagctaggaaatcca |
| | (SEQ ID NO:34) | (SEQ ID NO:35) |
| Lysyl | 5′-ttacccagccgaccaagatattcc | 5′-tcataacagccaggactcaatccc |
| oxidase | (SEQ ID NO:36) | (SEQ ID NO:37) |
| SIX2 | 5′-actgagtcttgaaccacagaaggg | 5′-acagaaggagagaatgaacggtgg |
| | (SEQ ID NO:38) | (SEQ ID NO:39) |
| HOXC6 | 5′-tcaattccaccgcctatgatccag | 5′-aatcctgagcgattgaggtctgtg |
| | (SEQ ID NO:40) | (SEQ ID NO:41) |
| 19ARF | 5′-atgggtcgcaggttcttggt | 5′-ctatgcccgtcggtctgggc |
| | (SEQ ID NO:42) | (SEQ ID NO:43) |
| GAPDH | 5′-gaaggtgaaggtcggagt | 5′-gaagatggtgatgggatttc |
| | (SEQ ID NO 44:) | (SEQ ID NO:45) |

Clonogenicity and Differentiation Assays

For the clonogenicity assay, cells were plated at 1 cell/well into a 96 well plate using an automated instrument (Clonecyte Accessory and FACSvantage: Becton-Dickinson, Lincoln Park, N.J.). The cells were incubated with complete culture medium for 10 days, stained with Crystal Violet, and colonies with diameters of 2 millimeters or greater counted. For the differentiation assay, the cells were incubated in complete culture medium for 9 days and medium was changed to either osteogenic medium (10⁻⁸ M dexamethasone/0.2 millimolar ascorbic acid/10 millimolar beta-glycerolphosphate; Sigma, St. Louis, Mo.), or adipogenic medium (0.5 micromolar hydrocortisone/0.5 millimolar isobutylmethylxanthine/60 micromolar indomethacin). The incubation was continued for 3 weeks with a change of medium every 4 days. The plates were stained with either 10% formalin fixed colonies with Alizarin Red (Sigma, St. Louis, Mo.) or Oil Red 0 (Fisher scientific, Pittsburgh, Pa.).

Microarray Analysis

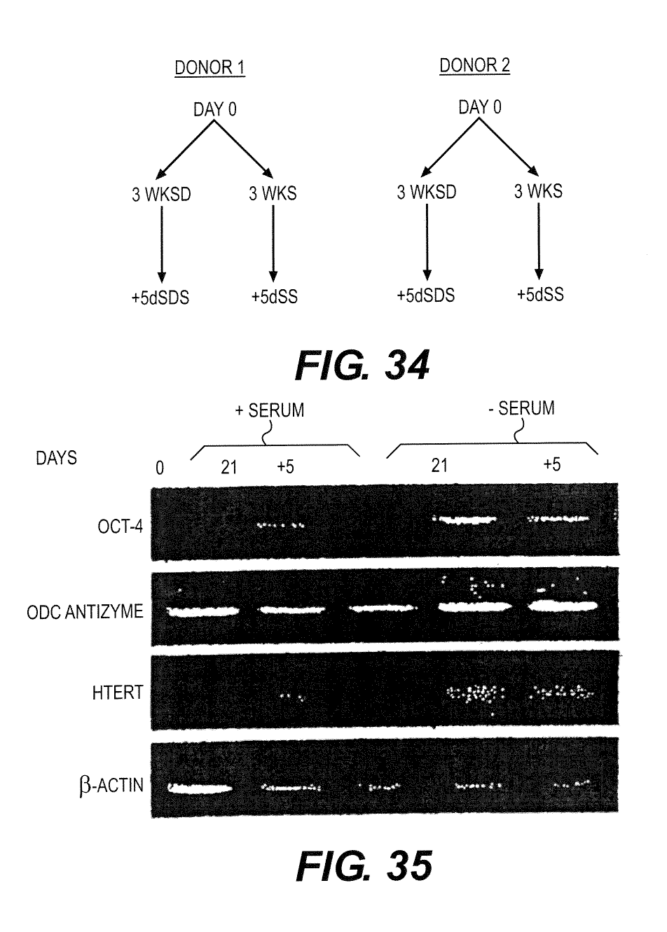

Total RNA was extracted (RNAeasy Kit; Qiagen, Valencia, Calif.), from 1×10⁶ cells of 5 samples from each of two donors as described in FIG. 34. The RNA expression was assayed with a chip containing probes for about 22,000 human genes (HGU133A array; Affymetrix, Santa Clara, Calif.). For the initial filtering for reproducibility of the data, the Microarray Suite 5.0 program (Affymetrix) was used to obtain signal intensities. The data were then filtered in the following steps: (a) genes that were not consistently scored as absent or present in the 3 wkS and 3 wkSD samples from both donors (FIG. 34) were eliminated; (b) genes scored as absent in all four samples were eliminated; (c) steps (a) and (b) were combined to reduce the number of genes to about 8,000; (d) genes in the four samples that did not show significant change from Day 0 (FIG. 34) were eliminated; (e) genes that did not show consistent scores of increase or decrease in the four samples were eliminated; (f) (d) and (e) were combined to reduce the number of genes to 915; (g) steps (e) and (f) were repeated for the four sample of +5dSDS and +5dSS and redundancies were eliminated to reduce the number of genes to 842. The hierarchical cluster analysis was carried out on the 842 genes with the dChip 1.3⁺ program (Li and Wong, 2001; http://biosunl.harvard.edu/complab/dchip/clustering.htm). Adjacent genes were merged if the cluster of merged genes maintained the same pattern of expression.

The Results of the experiments performed in this Example are now described.

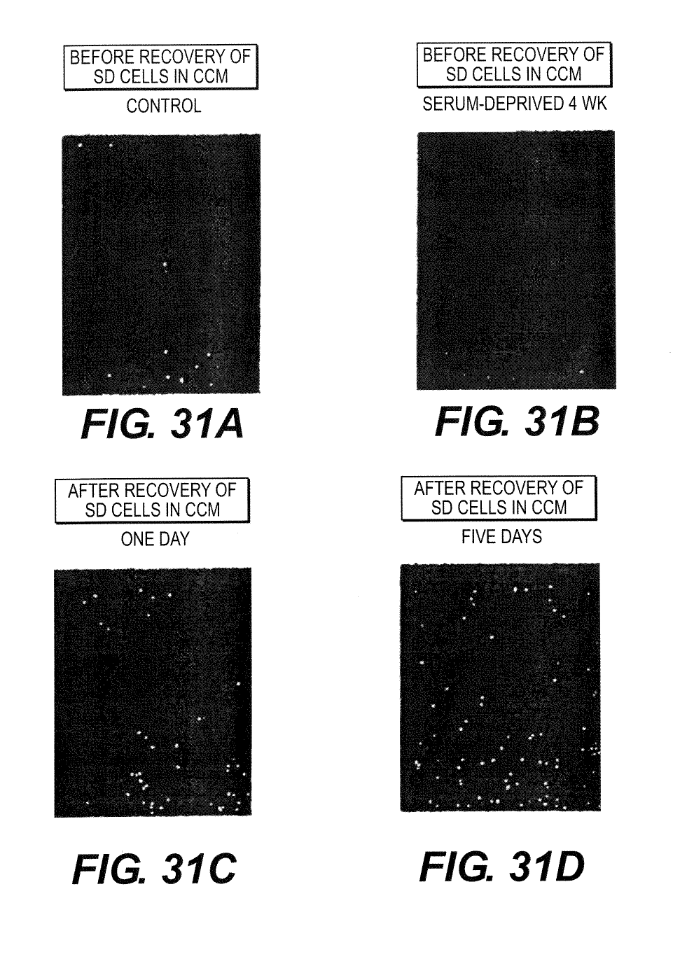

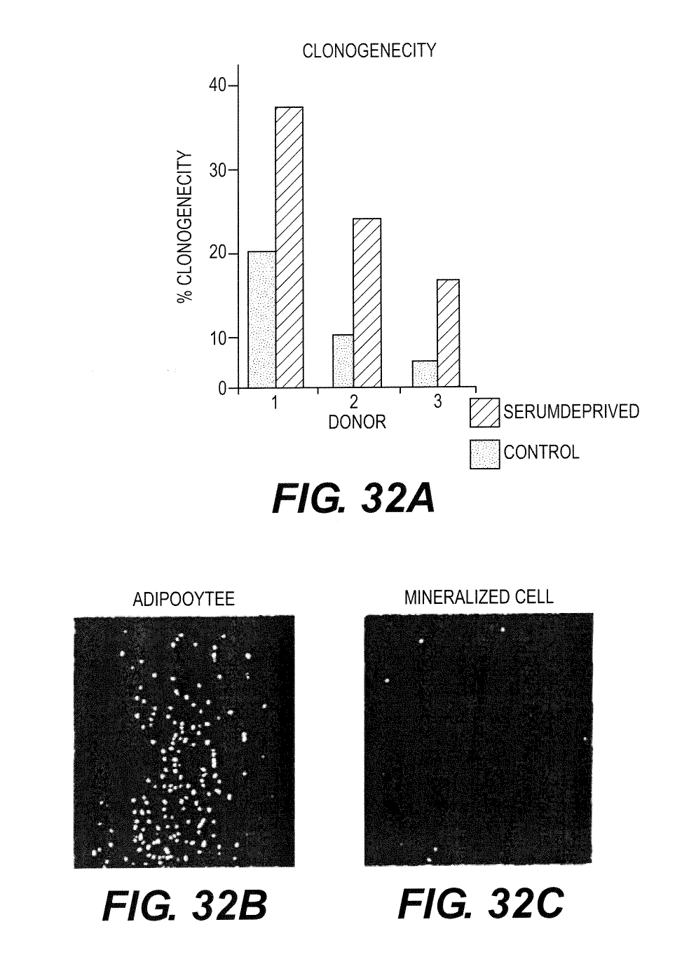

Initially, many of the cells in the serum-free medium appeared apoptotic and necrotic. Control cultures incubated in medium containing FCS became confluent. The serum-deprived cells (SD cells) were lifted with trypsin/EDTA, plated at 100 cells/cm² and incubated in medium containing FCS. After 5 days, the morphology of the SD cells changed from large, apparently senescent cells to the spindle-shaped cells characteristic of early passage hMSCs (FIG. 31). The replated cells (not shown) displayed a lag period of 4 to 5 days similar to the lag period seen when standard cultures of hMSCs are replated. Thereafter, the SD cells grew rapidly with a doubling time of about 24 hours for 4 to 5 days and until the cultures approached confluence. Cultures of SD cells continued to propagate through 13 passages. As noted previously (Colter et al., 2001; Sekiya et al., 2002), control cultures of hMSCs ceased to expand after 4 or 5 passages. The SD cells were more clonogenic than parallel samples incubated at the same densities in medium containing FCS and incubated for 4 weeks (FIG. 32A). The colonies formed were smaller than colonies formed by controls (not shown), but the SD cells retained their potential to differentiate into osteoblasts and adipocytes (FIG. 32B, 32C).

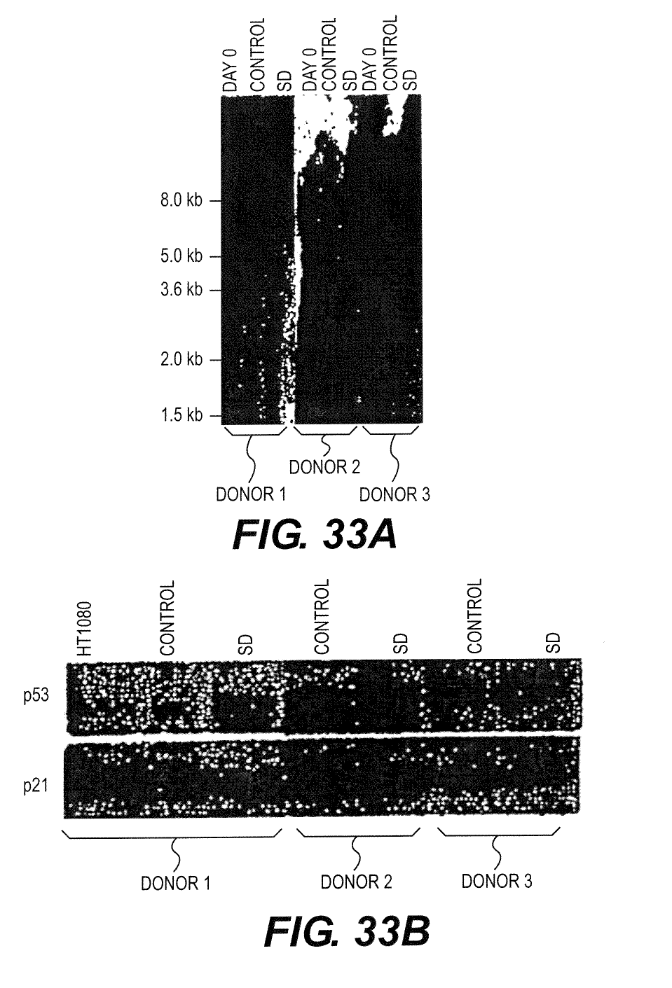

In assays of SD cells prepared from 15 different donors of bone marrow aspirates, the average telomere lengths were consistently longer than in the same cultures before serum-deprivation (FIG. 33A). Also, the average telomere lengths in the SD cells were longer than in control cells from the same hMSC preparations that were incubated in parallel in serum-containing medium. Assays for telomerase activity gave low and variable values for both SD cells and controls (not shown). The SD cells expressed p53 and p21 as assayed both by RT-PCR (not shown) and Western blot assays (FIG. 33B), an observation suggesting the cells were not transformed.

On the basis of these observations, analyses to test the hypothesis that the SD cells expressed a profile of genes more characteristic of early progenitors than the other cells in cultures of hMSCs was performed. Cells from two different donors were assayed under five different conditions: (1) initial plating of passage 2 hMSCs at low density (100 cells/cm²) and incubation for 5 days (Day 0 cells in FIG. 34) so that the cultures contained about equal proportions of RS cells and more mature cells; (2) incubation of the Day 0 samples in serum-free medium for 3 weeks (3 wk SD cells in FIG. 34); (3) incubation of parallel Day 0 samples in serum-containing complete medium for 3 weeks (3 wkS); (4) replating of 3wkSD samples in serum-containing medium for 5 days (5dSDS) so that the cells regained their original spindle-shaped morphology (FIG. 31); (5) replating of the 3 wkS samples in serum-containing medium for 5 days (5dSS).

RT-PCR assays (FIG. 35) indicated that mRNA levels were higher in SD cells than in controls for Oct-4, the catalytic subunit of telomerase (hTERT), and ornithine decarboxylase antizyme (ODC antizyme), three genes characteristically expressed in embryonic cells (Pesce and Scholer, 2001; Blackburn, 2001; Iwata et al. 1999).

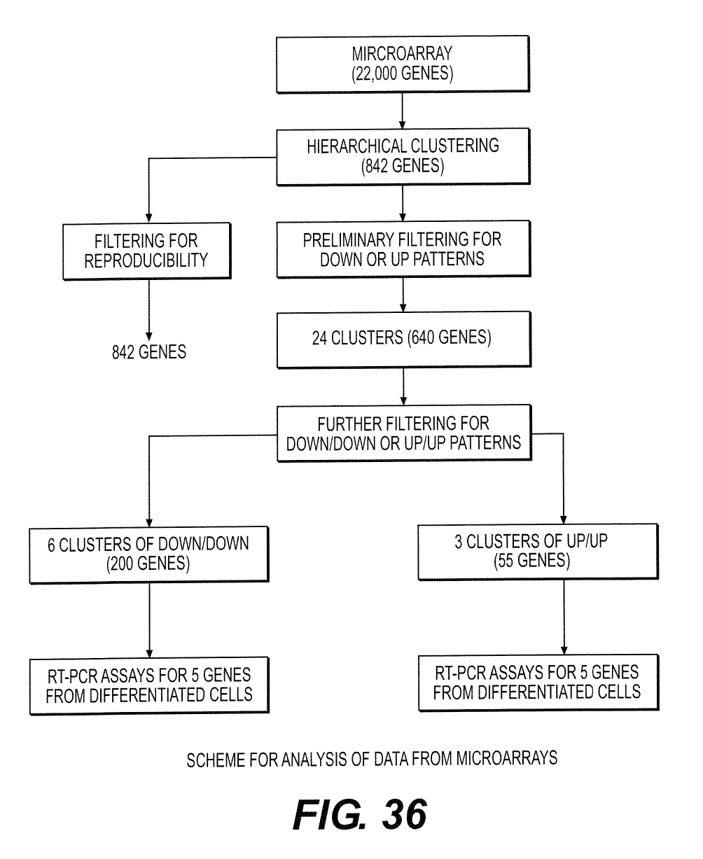

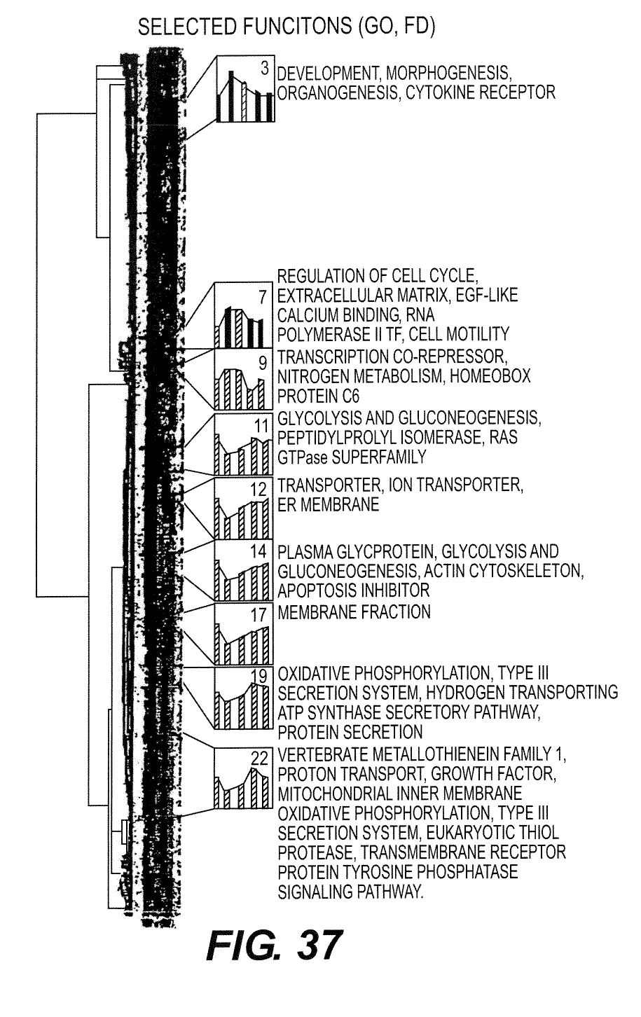

The same RNA samples were assayed by microarray and the changing patterns of gene expression analyzed by hierarchical clustering (Li and Wong, 2001). In brief (FIG. 36), the data from a chip containing about 22,000 genes were first filtered for reproducibility and significant changes to select 842 genes for further analysis. The 842 genes were assigned to hierarchical clusters with the dChip 1.3⁺ program (FIG. 37). The initial clusters were visually filtered to identify 24 clusters that showed distinctive patterns of either up regulation or down regulation in SD cells compared to the control cells. The 24 clusters were further filtered to identify (a) clusters in which genes were down regulated in response to serum deprivation and remained down regulated when the cells were returned to medium containing FCS (down/down pattern), and (b) three clusters in which genes were up regulated and remained up regulated (up/up pattern). Six down/down clusters (arbitrarily numbered 11, 12, 14, 17, 19, and 22), and three up/up clusters (numbers 3, 7 and 9) were identified. The functional annotations assigned to five of the six down/down clusters by the dChip program (FIG. 37) included genes encoding membrane fractions and membrane associated receptors or transporters. Two down/down clusters (clusters 11 and 14) also included genes for intermediary metabolism. One down/down cluster (cluster 14) contained a gene for an apoptosis inhibitor. The three up/up clusters included genes involved in development, morphogenesis, and organogenesis (cluster 3); genes involved in regulation of cell cycle, for a EGF-like calcium binding protein, RNA polymerase II transcription factor and cell motility (cluster 7); and genes for a transcription co-repressor, nitrogen metabolism and homeobox protein C6 (cluster 9).

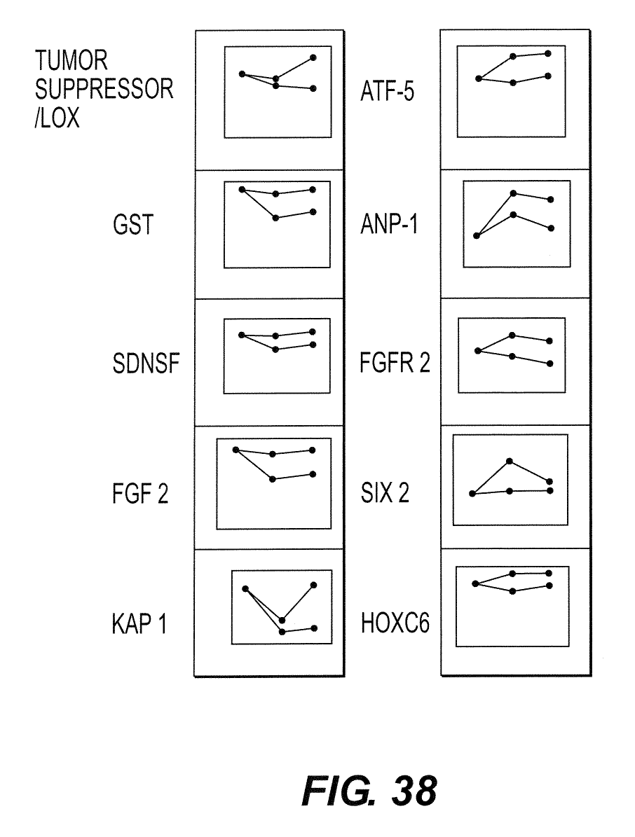

In the next step of analysis of the microarray data (FIG. 36), five individual genes from the down/down clusters that are expressed in differentiated cells and five genes from the up/up clusters that are expressed in uncommitted cells were examined in greater detail. The down/down genes (FIG. 38) included a tumor suppressor gene also referred to as lysyl oxidase because it encodes an enzyme that is required for the extracellular cross-linking of collagen and elastin; glutathione S transferase that is involved in the blood-barrier in brain and testes; neural stem cell derived neuronal survival protein; fibroblast growth factor-2; and keratin associated protein 1. The up/up genes (FIG. 38) included activating transcription factor 5 (ATF-5) that binds to the cAMP response elements in many promoters; angiopoietin-1 that promotes sprouting of endothelial cells; fibroblast growth factor-2 receptor; sine oculis; homeobox homolog 2; and homeobox C6 that belongs to the family of homeobox D4 genes involved in early development.

The results demonstrate that subjecting early passage hMSCs to serum deprivation for 2 to 4 weeks selects for a distinct sub-population of cells. The SD cells are remarkable in that they survive complete serum deprivation for prolonged periods of time, have long telomeres, and enhanced expression of genes expressed primarily in early progenitor cells. At the same time, the SD cells retained most of the characteristics of hMSCs in that they generated single-cell derived colonies and differentiated into both osteoblasts and adipocytes. SD cells were obtained from 75% of early passage hMSCs obtained from over 30 separate donors of marrow aspirates.

The yield of SD cells decreased markedly with passage number so that they could not be isolated from hMSCs preparations after 3 passages (not shown). Therefore SD cells were probably not present in significant numbers in the hMSC preparations used in most previous experiments. In comparison to the hierarchy of hematopoietic system (Wagers et al., 2002), RS cells that were previously identified as a rapidly self-renewing sub-population in hMSC cultures (Colter et al., 2001) are probably comparable to transitory amplifying cells. SD cells are more slowly replicating earlier progenitors and therefore more closely resemble hematopoietic stem cells or partially committed hematopoietic stem cells.

### Example 7

### Inhibitors of Dkk-1

Mesenchymal stem cells or marrow stromal cells (hMSCs) can differentiate into numerous mesenchymal tissue lineages including osteoblasts, chondrocytes, adipocytes, and neural precursors making them attractive candidates for cytotherapy, bioengineering, and gene therapy (Prockop, 1997 Science 276:263-272). Synthesis of the canonical Wnt inhibitor Dkk-1 is required before the cells enter the cell cycle. It has been demonstrated that canonical Wnt signaling plays a positive role in hMSC growth osteogenesis. In addition to its proliferative properties, canonical Wnt signaling has been implicated in a variety of developmental processes. For example, Wnt signaling plays an essential role in the differentiation of C₃H₁₀T1/2 cells to osteoblasts (Bain et al. 2003 Biochem. Biophys. Res. Commun. 301:84-91).

It has been demonstrated that the mechanism of hMSC differentiation is aberrant in the case of multiple myeloma (MM), a plasma cell malignancy. In the case of MM, the tumor cells synthesize and secrete high levels of Dkk-1 that prevent differentiation of hMSCs into osteoblasts. In association with other complications, bone turnover is compromised resulting in the breakdown of the skeleton. These osteolytic lesions per se contribute significantly to the pathology of MM. Inhibition of the action of Dkk-1 in inhibiting the differentiation of hMSCs into osteoblasts can be used as therapy for the reduction of osteolysis in MM-affected individuals.

The molecular determinants of osteolytic lesions in multiple myeloma (MM) patients has recently been identified using oligonucleotide microarray profiling. Of the genes found to be reproducibly over-expressed in cases of MM exhibiting osteolytic lesions, Dkk-1 was identified to be a secreted product. Further investigation strongly correlated Dkk-1 serum levels with MRI-diagnosed osteolytic lesions and demonstrated that Dkk-1 could reduce the bone morphogenic protein 2 (BMP2)—induced alkaline phosphatase activity in osteoblast progenitor cells. While not wishing to be bound to any particular theory, it seems the abnormally high level of Dkk-1 prevents Wnt-mediated terminal differentiation of progenitors into osteoblasts. In support of these observations, a different study has demonstrated that canonical Wnt signaling is responsible for BMP2-mediated differentiation of osteoblast progenitor cell lines and Dkk-1 inhibits this process. Glycogen synthetase kinase 3β (GSK3β) inhibition is the key molecular effect of Wnt signaling that results in osteogenesis. Therefore, based upon the present disclosure, a direct inhibitor of GSK3β or a composition that inhibits Dkk-1 binding to a corresponding receptor would inhibit the effects of Dkk-1 at the membrane and thus would prevent phosphorylation of β-catenin and prevent the degradation of β-catenin. The overall result is an increase in the rate of osteogenesis and improvement of osteolytic lesion repair.

Peptide Derived from Dkk-1 (Peptide A) Alleviates Dkk-1 Mediated Inhibition of Osteogenesis.

Various peptides, including but not limited to peptides A-G; SEQ ID Nos:11-17, were synthesized using standard protocols by the Tufts University Medical School Core Facility (Boston, Mass.) using an ABI 431 Peptide synthesizer employing FastMoc chemistry. The peptides were biotinylated at the amino terminus and purified by reverse phase high performance liquid chromatography. To confirm purity and identity, the peptides were subjected to matrix-assisted laser desorbtion ionization time of flight (MALDI-TOF) mass spectrometry (Ciphergen Chip Reader, Ciphergen Biosystems, Freemont, Calif.).

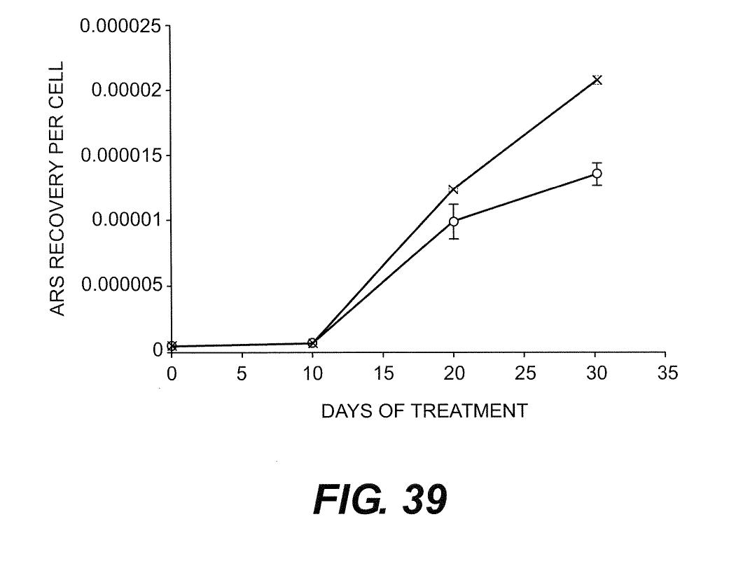

To determine the effects of peptide A on the effects of Dkk-1 and osteogenic differentiation, cells were plated at 1000 cells per cm² in 6 well plates with complete medium and allowed to adhere for 15 hr. The next day, the complete medium was replaced with osteogenic medium containing 50 μg mL¹ peptide A (SEQ ID NO:11) or 50 μg mL¹ vehicle as a control. At 7 day intervals, the hMSCs were recovered from 3 wells of a 6 well plate and the number of cells was assayed. Another 3 wells were fixed and stained with Alizarin Red S for dye extraction and quantification of mineralization. The level of Alizarin Red S staining per cell was calculated and plotted for 21 days. In this assay, peptide A increased the rate of osteogenic differentiation by hMSCs when compared with a control without the presence of peptide A, thereby providing evidence that peptide A inhibits the effect of Dkk-1 on canonical Wnt signaling during osteogenesis (FIG. 39).

Lithium Alleviates Dkk-1 Mediated Inhibition of Osteogenesis.

Lithium inhibits the degradation of β-catenin that is detected in response to Dkk-1 activity. Even in the presence of vast levels of Dkk-1, the downstream components of the pathway are inhibited, thus negating the effect of Dkk-1 at the membrane. The addition of lithium to cells at the log phase of hMSC growth reduced the rate of proliferation in a dose-dependent manner and resulted in an overall increase in the level of cytosolic β-catenin.

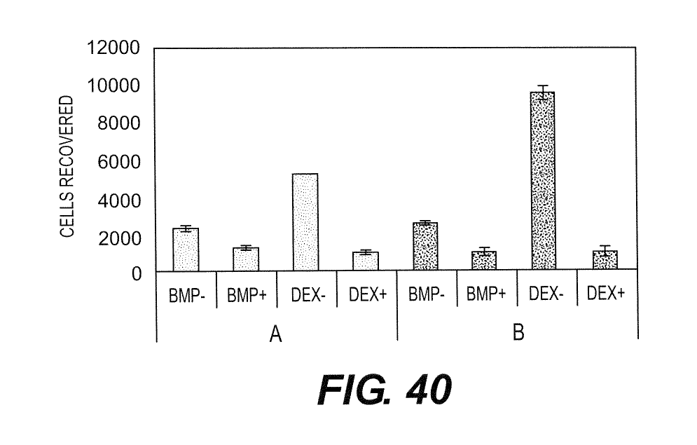

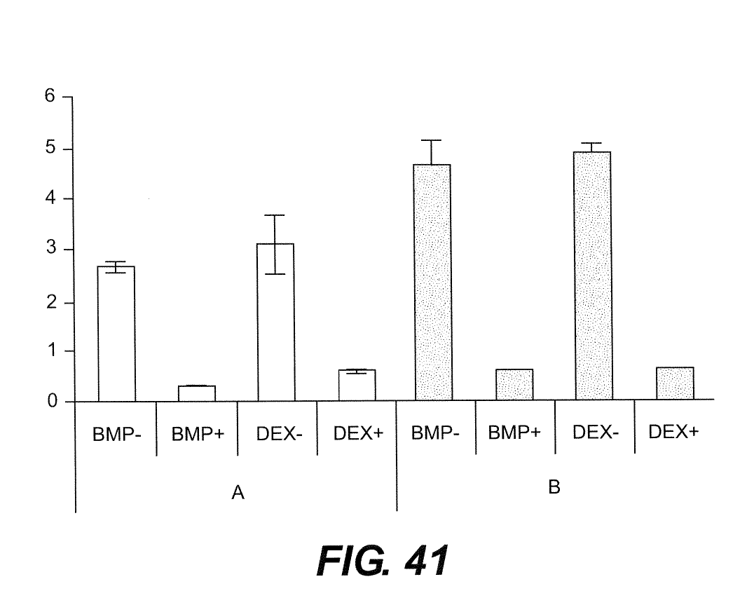

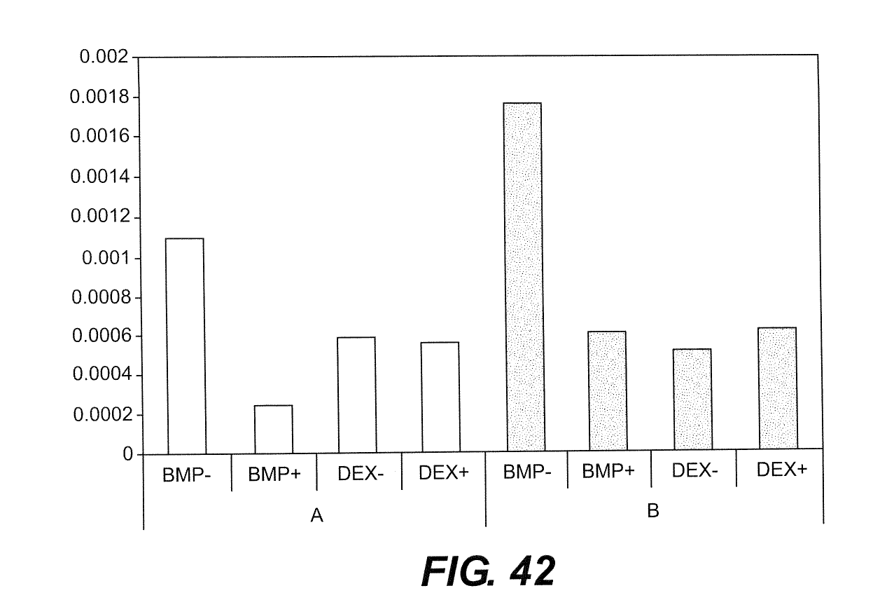

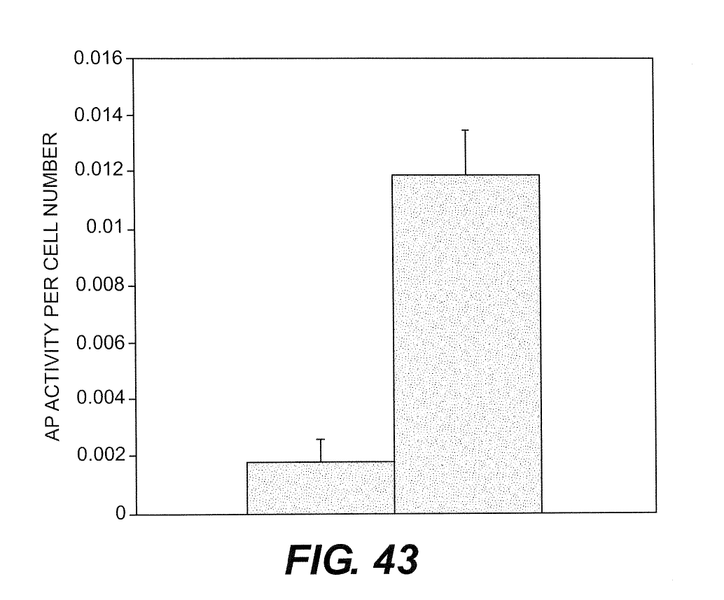

The osteogenic potential of MSCs in the presence or absence of Dkk-1 was investigated. MSCs were differentiated to an osteogenic phenotype using two standard protocols; one mediated by dexamethasone and one mediated by bone morphogenic protein 2 (BMP2). In the presence of Dkk-1 (100 ng mL¹), MSCs underwent extensive apoptosis in both of the osteo-inductive conditions tested (FIG. 40). As a result of the apoptosis, the overall net activity of the osteogenic marker, alkaline phosphatase (ALP) per culture was significantly reduced (FIG. 41). The level of ALP production per surviving cell remained constant in the case of Dex-induced mineralization (FIG. 42), but varied with donor in the case of BMP2 induction. For the two donors tested, Dkk-1 reduced the level of ALP production per surviving cell and in one donor, the surviving cells appeared to compensate for the loss of a major fraction of the monolayer by up-regulating ALP production (FIG. 43). In any event, the overall effect of Dkk-1 treatment was to reduce the net production of alkaline phosphatase.

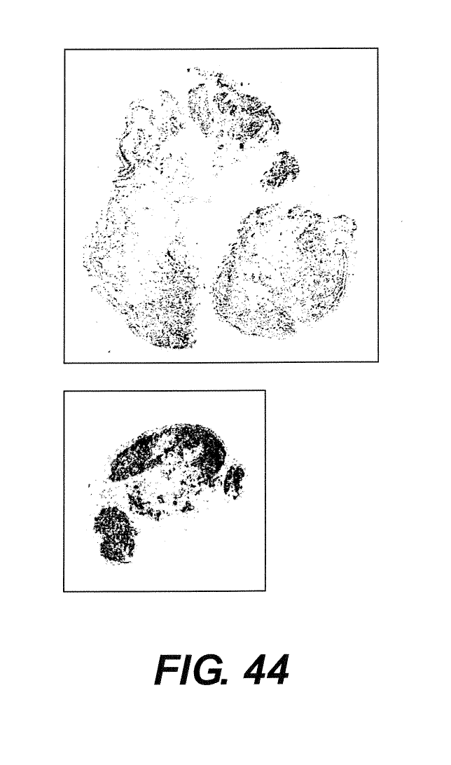

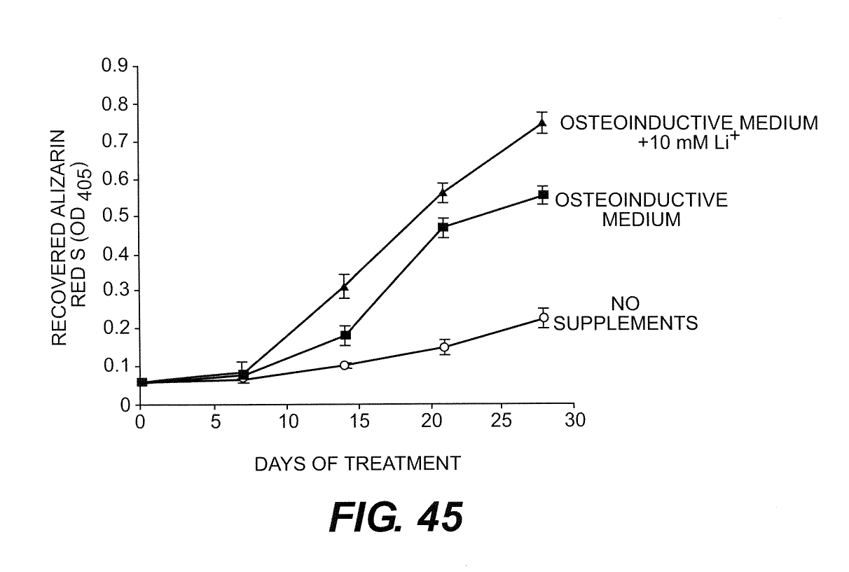

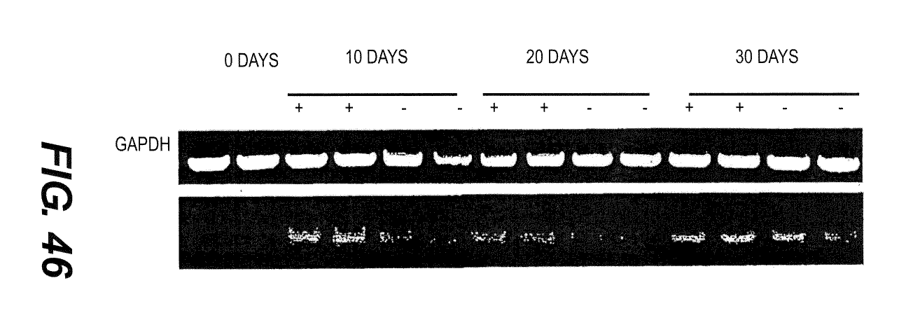

The effect of LiCl on osteogenesis in an osteogenic differentiation assay was assessed. Lithium has been known to inhibit GSK3β, a critical enzyme in the canonical Wnt pathway. Not wishing to be bound to any particular theory, inhibition of GSK3β has the opposite effect of Dkk-1 in present system. To investigate the effect of lithium directly on osteogenesis, the hMSCs were grown to confluency and treated for up to 30 days with an osteogenic medium containing a 100 fold lower than standard concentration of dexamethasone and 10 mM LiCl or 10 mM KCl. Lower levels of dexamethasone were used to improve the detection of differences in osteogenesis induced by LiCl. Under these conditions, the hMSC monolayer detached from the plastic and spontaneously curled into a roughly spherical cellular aggregate. In the presence of LiCl, the aggregate was formed after about 7 days of treatment whereas in the presence of KCl, the effect was seen after about 12 days. The aggregates were fixed, paraffin embedded, sectioned and stained for calcified deposits (Alizarin Red S). On sectioning and staining of the sections with Alizarin Red S, it was apparent that mineralization was much more evident in the LiCl treated aggregates than in the control with dense patches of mineral detected throughout the sections of the LiCl-treated cells (FIG. 44). Quantification of mineral by colorimetric measurement of Alizarin Red S staining demonstrated that LiCl treated cultures produced mineralized matrix more rapidly than the controls (FIG. 45).

Analysis of gene expression by the differentiating hMSCs revealed that transcripts commonly associated with osteogenesis increased over time in osteogenic medium both in the presence and absence of LiCl but there was a striking up-regulation of alkaline phosphatase transcription. Alkaline phosphatase transcription was maximal after 10 days in lithium treated cultured compared with 30 days in the controls (FIG. 46).

GSK3β Inhibitors for the Treatment of Osteolytic Lesions in Multiple Myeloma.

Multiple myeloma (MM) is a uniformly fatal malignancy of antibody-secreting plasma cells (PCs). In about 80% of patients, painful osteolytic lesions accompany the malignancy. Activation of the canonical Wnt pathway leads to the inhibition of glycogen synthetase kinase-3 (GSK3β), resulting in an increase in cytosolic β-catenin that regulates gene expression and drives hMSCs to an osteogenic phenotype. It has been demonstrated that the canonical Wnt signaling antagonists, Dkk-1 and Frizzled related protein B (FrzB) contribute to the inhibition of Wnt-mediated hMSC differentiation. MM cells express high levels of these inhibitors in patients and also upregulate the formation of osteoclasts. Up-regulation of osteoclast activity coupled with the inhibition of osteogenesis is therefore a cause of osteolytic lesion formation.

It has been demonstrated that when MM cells are co-cultured with bone marrow hMSCs, MM cells up-regulate their expression of Dkk-1, while initial studies indicate a decrease in FrzB. Furthermore, IL-6 is produced at high levels by hMSCs, and induces rapid MM proliferation, as well as being a potent inducer of osteoclast activity. These observations demonstrate a cycle of osteogenic inhibition by Dkk-1/FrzB and perpetuation of the plasma cell malignancy through IL-6 induced proliferation in the cell lines tested.

Therapy directed at the inhibition of GSK3β may provide an agent to promote osteoblast differentiation. Inhibitors of GSKβ that mimic positive Wnt signaling was tested to assess the effects of GSK3β inhibitors to promote osteoblast differentiation. With the differentiation of into osteoblast using a GSK3β inhibitor, GSK3β inhibitors can be used to prevent osteolytic lesions that are the major cause of morbidity in MM.

Multiple myeloma patients have high levels of circulating Dkk-1 due to the high levels of expression by the malignant plasma cells. The plasma cells and mesenchymal cells also express interleukin 6 that exacerbates the malignancy and activated osteoclasts which break down bone. Dkk-1 prevents repair of the broken down bone by killing MSCs (osteoblast precursor cells) and also by reducing their ability to differentiate. Osteoblast differentiation is driven by a signal transduction pathway mediated by the Wnt class of secreted ligands; a major step in this signal transduction pathway is the inhibition of glycogen synthetase kinase 3β (GSK3β). Dkk-1 inhibits this pathway at the membrane and prevents inhibition of GSK3β and thus Dkk-1 inhibits osteogeneic differentiation. Two classes of molecules can prevent the effect of Dkk-1: (1) molecules that compete for Dkk-1 binding at the level of the membrane (peptide analogs of Dkk-1) or (2) molecules that inhibit GSK3β directly (e.g. lithium carbonate, lithium chloride, indirubin oxime classes of molecules).

It has been demonstrated that Dkk-1 inhibits osteogenesis of MSCs at two levels; apoptosis and direct inhibition of differentiation. It has also been demonstrated that the GSK3β inhibitor, lithium chloride, and a putative antagonist of Dkk-1 (peptide A) enhances osteogenic differentiation. Therefore either class of molecule can be used in treating osteolytic lesion formation in multiple myeloma.

### Example 8

### Wnt/β-Catenin Signaling is Required for Osteoblastic Differentiation of hMSCs

Human mesenchymal stem cells (hMSCs) from bone marrow stromal differentiate into mesenchymal tissue lineages and are good candidates for cellular therapies. Given that loss-of-function mutations in the Wnt receptor LRP5 result in a low bone mass phenotype (Gong et al., 2001 Cell 107:513-523), and a gain-of-function mutation in the same receptor results in high bone mass (Boyden et al., 2002 N. Engl. J. Med. 346:1513-1521), it has been demonstrated that Wnt signaling is required for osteoblastic differentiation of hMSCs. Furthermore, the Wnt antagonist Dkk-1 inhibits this differentiation and predisposes the cells towards cell cycle entry.

A functional Wnt signaling pathway exists in hMSCs, which express Lrp6, Wnt5a, and β-catenin. While not wishing to be bound to any particular theory, the data disclosed elsewhere herein depicts an ex vivo model of osteogenesis, in which BMP2 induces expression of Wnt ligands that signals in an autocrine fashion to promote osteogenesis. Accordingly, alkaline phosphatase levels are highly upregulated in cells treated with BMP2, compared to controls, whereas no increase is seen when the treatment includes Dkk-1.

Dkk-1 has been implicated in forming osteolytic bone lesions in multiple myeloma (MM), thus secreted Wnt inhibitors and pharmacological Wnt activators can be used to examine the role of Wnt signaling in the pathology of MM. Treatment with 6-bromoindirubin oxime, PS-341, or lithium chloride maintains alkaline phosphatase expression in the presence of Dkk-1. Therefore, this model of osteogenesis provides an experimentally accessible means to screen for novel drugs which activate Wnt signaling in hMSCs and therefore represents important treatments for bone disease and cancer.

It will be apparent to those skilled in the art that various modifications and variations can be made in the methods and compositions of the present invention without departing from the spirit or scope of the invention. Thus, it is intended that the present invention cover the modifications and variations of the present invention provided they come within the scope of the appended claims and their equivalents.

1. A method of treating an osteolytic lesion in a mammal comprising administering to a mammal in need thereof an effective amount of a Dkk-1 antagonist, wherein said Dkk-1 antagonist inhibits Dkk-1 activity on the Wnt signaling pathway.

2-25. (canceled)


SEQUENCE LISTING (53 sequences)

SEQ ID NO: 1 - protein, 15 aa, Artificial
  Peptide corresponding to the second Cys-rich domain of Dkk-1 protein
ARHFWSKICKPVLKE

SEQ ID NO: 2 - DNA, 33 bp, Artificial
  Forward primer for Dkk-1
ccttctcatatgatggctctgggcgcagcggga

SEQ ID NO: 3 - DNA, 33 bp, Artificial
  Reverse primer for Dkk-1
cctggaggtttagtgtctctgacaagtgtggaa

SEQ ID NO: 4 - DNA, 20 bp, Artificial
  Forward primer for GAPDH
ccccttcattgacctcaact

SEQ ID NO: 5 - DNA, 20 bp, Artificial
  Reverse primer for GAPDH
cgaccgtaacgggagttgct

SEQ ID NO: 6 - DNA, 21 bp, Artificial
  Forward primer for LRP-6
ccacaggccaccaatacagtt

SEQ ID NO: 7 - DNA, 24 bp, Artificial
  Reverse primer for LRP-6
tccggaggagtctgtacagggaga

SEQ ID NO: 8 - DNA, 21 bp, Artificial
  Biotinylated at 5′ end oligonucleotide for ELISA detection of Dkk-1
atagcaccttggatgggtatt

SEQ ID NO: 9 - DNA, 21 bp, Artificial
  Biotinylated at 5′ end oligonucleotide for ELISA detection of GAPDH
catgccatcactgccacccag

SEQ ID NO: 10 - protein, 108 aa, Artificial
  Dkk-1 LRP binding domain
GNDHSTLDGYSRRTTLSSKMYHTKGQEGSVCLRSSDCASGLCCARHFWSKICKPVLKEGQVCTKHRRKGSHGLEIFQRCYCGEGLSCRIQKDHHQASNSSRLHTCQRH

SEQ ID NO: 11 - protein, 20 aa, Artificial
  Peptide synthesized from Dkk-1 LRP-6 binding domain
GNDHSTLDGYSRRTTLSSKM

SEQ ID NO: 12 - protein, 20 aa, Artificial
  Peptide synthesized from Dkk-1 LRP-6 binding domain
LSSKMYHTKGQEGSVCLRSS

SEQ ID NO: 13 - protein, 20 aa, Artificial
  Peptide synthesized from Dkk-1 LRP-6 binding domain
SLRSSDCASGLCCARHFWSK

SEQ ID NO: 14 - protein, 20 aa, Artificial
  Peptide synthesized from Dkk-1 LRP-6 binding domain
FWSKICKPVLKEGQVCTKHR

SEQ ID NO: 15 - protein, 20 aa, Artificial
  Peptide synthesized from Dkk-1 LRP-6 binding domain
STKHRRKGSHGLEIFQRCYS

SEQ ID NO: 16 - protein, 20 aa, Artificial [sequence omitted]

SEQ ID NO: 17 - protein, 17 aa, Artificial
  Peptide synthesized from Dkk-1 LRP-6 binding domain
DHHQASNSSRLHTCQRH

SEQ ID NO: 18 - DNA, 20 bp, Artificial
  Forward primer for Oct-4
cccccgccgtatgagttctg

SEQ ID NO: 19 - DNA, 23 bp, Artificial
  Reverse primer for Oct-4
tgtgttcccaattccttccttag

SEQ ID NO: 20 - DNA, 20 bp, Artificial
  forward primer for hTERT
cgctggtggcccagtgcctg

SEQ ID NO: 21 - DNA, 20 bp, Artificial
  Reverse primer for hTERT
ctcgcacccggggctggcag

SEQ ID NO: 22 - DNA, 20 bp, Artificial
  Forward primer for OCT-4
cgctccggcccacaaatctc

SEQ ID NO: 23 - DNA, 20 bp, Artificial
  Reverse primer for OCT-4
ccgcacgacaaccgcaccat

SEQ ID NO: 24 - DNA, 20 bp, Artificial
  Forward primer for ODC antizyme
ccgcacgacaaccgcaccat

SEQ ID NO: 25 - DNA, 20 bp, Artificial
  Reverse primer for ODC antizyme
cgctccggcccacaaatctc

SEQ ID NO: 26 - DNA, 24 bp, Artificial
  Forward primer for ATF-5
aaggagctggaacagatggaagac

SEQ ID NO: 27 - DNA, 24 bp, Artificial
  Reverse primer for ATF-5
ttgtaaacctcgatgagcaggtcc

SEQ ID NO: 28 - DNA, 24 bp, Artificial
  Forward primer for FGF-2
gtgtgctaaccgttacctggctat

SEQ ID NO: 29 - DNA, 24 bp, Artificial
  Reverse primer for FGF-2
aggtaagcttcactgggtaacagc

SEQ ID NO: 30 - DNA, 24 bp, Artificial
  Forward primer for FGF2R
tgtgctaaccgttacctggctatg

SEQ ID NO: 31 - DNA, 24 bp, Artificial
  Reverse primer for FGF2R
aggtaagcttcactgggtaacagc

SEQ ID NO: 32 - DNA, 24 bp, Artificial
  Forward primer for GST
tgggaagaacaagatcacccagag

SEQ ID NO: 33 - DNA, 24 bp, Artificial
  Reverse primer for GST
gttgtccaggtagctcttccaagt

SEQ ID NO: 34 - DNA, 24 bp, Artificial
  Forward primer for KAP1
acccaaccttcagatcaactcctg

SEQ ID NO: 35 - DNA, 24 bp, Artificial
  Reverse primer for KAP1
ccggttgagaagctaggaaatcca

SEQ ID NO: 36 - DNA, 24 bp, Artificial
  Forward primer for lysyl oxidase
ttacccagccgaccaagatattcc

SEQ ID NO: 37 - DNA, 24 bp, Artificial
  Reverse primer for lysyl oxidase
tcataacagccaggactcaatccc

SEQ ID NO: 38 - DNA, 24 bp, Artificial
  Forward primer for SIX2
actgagtcttgaaccacagaaggg

SEQ ID NO: 39 - DNA, 24 bp, Artificial
  Reverse primer for SIX2
acagaaggagagaatgaacggtgg

SEQ ID NO: 40 - DNA, 24 bp, Artificial
  Forward primer for HOXC6
tcaattccaccgcctatgatccag

SEQ ID NO: 41 - DNA, 24 bp, Artificial
  Reverse primer for HOXC6
aatcctgagcgattgaggtctgtg

SEQ ID NO: 42 - DNA, 20 bp, Artificial
  Forward primer for 19ARF
atgggtcgcaggttcttggt

SEQ ID NO: 43 - DNA, 20 bp, Artificial
  Reverse primer for 19ARF
ctatgcccgtcggtctgggc

SEQ ID NO: 44 - DNA, 18 bp, Artificial
  Forward primer for GAPDH
gaaggtgaaggtcggagt

SEQ ID NO: 45 - DNA, 20 bp, Artificial
  Reverse primer for GAPDH
gaagatggtgatgggatttc

SEQ ID NO: 46 - DNA, 324 bp, Artificial
  Dkk-1 LRP binding domain
ggtaatgatcatagcaccttggatgggtattccagaagaaccaccttgtcttcaaaaatgtatcacaccaaaggacaagaaggttctgtttgtctccggtcatcagactgtgcctcaggattgtgttgtgctagacacttctggtccaagatctgtaaacctgtcctgaaagaaggtcaagtgtgtaccaagcataggagaaaaggctctcatggactagaaatattccagcgttgttactgtggagaaggtctgtcttgccggatacagaaagatcaccatcaagccagtaattcttctaggcttcacacttgtcagagacac

SEQ ID NO: 47 - DNA, 60 bp, Artificial
  DNA sequence to Peptide A
ggtaatgatcatagcaccttggatgggtattccagaagaaccaccttgtcttcaaaaatg

SEQ ID NO: 48 - DNA, 60 bp, Artificial
  DNA sequence for Peptide B
ttgtcttcaaaaatgtatcacaccaaaggacaagaaggttctgtttgtctccggtcatca

SEQ ID NO: 49 - DNA, 60 bp, Artificial
  DNA sequence for Peptide C
  misc_feature: nnn could be tct, tcc, tca, tcg or agt
  misc_feature (1)..(3): n is a, c, g, or t
nnnctccggtcatcagactgtgcctcaggattgtgttgtgctagacacttctggtccaag

SEQ ID NO: 50 - DNA, 60 bp, Artificial
  DNA sequence for Peptide D
ttctggtccaagatctgtaaacctgtcctgaaagaaggtcaagtgtgtaccaagcatagg

SEQ ID NO: 51 - DNA, 60 bp, Artificial
  DNA sequence of Peptide E
  misc_feature: nnn could be tct, tcc, tca, tcg or agt
  misc_feature (1)..(3): n is a, c, g, or t
  misc_feature (58)..(60): n is a, c, g, or t
nnnaccaagcataggagaaaaggctctcatggactagaaatattccagcgttgttacnnn

SEQ ID NO: 52 - DNA, 60 bp, Artificial
  DNA sequence for Peptide F
  misc_feature: nnn could be tct, tcc, tca, tcg or agt
  misc_feature (13)..(15): n is a, c, g, or t
cagcgttgttacnnnggagaaggtctgtcttgccggatacagaaagatcaccatcaagcc

SEQ ID NO: 53 - DNA, 51 bp, Artificial
  DNA sequence for Peptide G
gatcaccatcaagccagtaattcttctaggcttcacacttgtcagagacac